SEQUENCE LISTING (373 sequences)

SEQ ID NO: 1 - protein, 18 aa, Artificial
  Note  Artificial = synthetic construct
TQEFWDNLEKETEGLRQE

SEQ ID NO: 2 - protein, 18 aa, Artificial
  Note  Artificial = synthetic construct
EQRLGETEKELNDWFEQT

SEQ ID NO: 3 - protein, 18 aa, Artificial
  Note  Artificial = synthetic construct
GEEMRDRARAHVDALRTH

SEQ ID NO: 4 - protein, 18 aa, Artificial
  Note  Artificial = synthetic construct
HTRLADVHARARDRMEEG

SEQ ID NO: 5 - protein, 18 aa, Artificial
  Note  Artificial = synthetic construct
SDELRQRLAARLEALKEN

SEQ ID NO: 6 - protein, 18 aa, Artificial
  Note  Artificial = synthetic construct
NEKLAELRAALRQRLEDS

SEQ ID NO: 7 - protein, 18 aa, Artificial
  Note  Artificial = synthetic construct
LAEYHAKATEHLSTLSEK

SEQ ID NO: 8 - protein, 18 aa, Artificial
  Note  Artificial = synthetic construct
KESLTSLHETAKAHYEAL

SEQ ID NO: 9 - protein, 18 aa, Artificial
  Note  Artificial = synthetic construct
LESAKVSALSALEEATKK

SEQ ID NO: 10 - protein, 18 aa, Artificial
  Note  Artificial = synthetic construct
KKTAEELASLASVKASEL

SEQ ID NO: 11 - protein, 5 aa, Artificial
  Note  Artificial = synthetic construct
AKPVL

SEQ ID NO: 12 - protein, 5 aa, Artificial
  Note  Artificial = synthetic construct
LVPKA

SEQ ID NO: 13 - protein, 4 aa, Artificial
  Note  Artificial = synthetic construct
KPVL

SEQ ID NO: 14 - protein, 6 aa, Artificial
  Note  Artificial = synthetic construct
PVLPVP

SEQ ID NO: 15 - protein, 4 aa, Artificial
  Note  Artificial = synthetic construct
AKPV

SEQ ID NO: 16 - protein, 3 aa, Artificial
  Note  Artificial = synthetic construct
AKP

SEQ ID NO: 17 - protein, 3 aa, Artificial
  Note  Artificial = synthetic construct
KPV

SEQ ID NO: 18 - protein, 5 aa, Artificial
  Note  Artificial = synthetic construct
KLSPL

SEQ ID NO: 19 - protein, 4 aa, Artificial
  Note  Artificial = synthetic construct
LSPL

SEQ ID NO: 20 - protein, 3 aa, Artificial
  Note  Artificial = synthetic construct
SPL

SEQ ID NO: 21 - protein, 5 aa, Artificial
  Note  Artificial = synthetic construct
LPSLK

SEQ ID NO: 22 - protein, 4 aa, Artificial
  Note  Artificial = synthetic construct
LPSL

SEQ ID NO: 23 - protein, 3 aa, Artificial
  Note  Artificial = synthetic construct
LPS

SEQ ID NO: 24 - protein, 18 aa, Artificial
  Note  Artificial = synthetic construct
LESFKVSFLSALEEYTKK

SEQ ID NO: 25 - protein, 18 aa, Artificial
  Note  Artificial = synthetic construct
KKTYEELASLFSVKFSEL

SEQ ID NO: 26 - protein, 9 aa, Artificial
  Note  Artificial = synthetic construct
PRGGSVLVT

SEQ ID NO: 27 - protein, 9 aa, Artificial
  Note  Artificial = synthetic construct
TVLVSGGRP

SEQ ID NO: 28 - protein, 6 aa, Artificial
  Note  Artificial = synthetic construct
PRGGSV

SEQ ID NO: 29 - protein, 6 aa, Artificial
  Note  Artificial = synthetic construct
TVLVSG

SEQ ID NO: 30 - protein, 3 aa, Artificial
  Note  Artificial = synthetic construct
PKA

SEQ ID NO: 31 - protein, 5 aa, Artificial
  Note  Artificial = synthetic construct
ALSPL

SEQ ID NO: 32 - protein, 5 aa, Artificial
  Note  Artificial = synthetic construct
LPSLA

SEQ ID NO: 33 - protein, 6 aa, Artificial
  Note  Artificial = synthetic construct
WRWWWW

SEQ ID NO: 34 - protein, 6 aa, Artificial
  Note  Artificial = synthetic construct
WWWWRW

SEQ ID NO: 35 - protein, 4 aa, Artificial
  Note  Artificial = synthetic construct
LNTQ

SEQ ID NO: 36 - protein, 4 aa, Artificial
  Note  Artificial = synthetic construct
QTNL

SEQ ID NO: 37 - protein, 6 aa, Artificial
  Note  Artificial = synthetic construct
LAKPVL

SEQ ID NO: 38 - protein, 6 aa, Artificial
  Note  Artificial = synthetic construct
LVPKAL

SEQ ID NO: 39 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 40 - protein, 41 aa, Artificial
  Note  Artificial = synthetic construct
TQEFWDNLEKETEGLRQEAKPVLLESFKVSFLSALEEYTKK

SEQ ID NO: 41 - protein, 41 aa, Artificial
  Note  Artificial = synthetic construct
TQEFWDNLEKETEGLRQELVPKALESFKVSFLSALEEYTKK

SEQ ID NO: 42 - protein, 40 aa, Artificial
  Note  Artificial = synthetic construct
TQEFWDNLEKETEGLRQEKPVLLESFKVSFLSALEEYTKK

SEQ ID NO: 43 - protein, 39 aa, Artificial
  Note  Artificial = synthetic construct
TQEFWDNLEKETEGLRQEPVLLESFKVSFLSALEEYTKK

SEQ ID NO: 44 - protein, 38 aa, Artificial
  Note  Artificial = synthetic construct
TQEFWDNLEKETEGLRQEPVLESFKVSFLSALEEYTKK

SEQ ID NO: 45 - protein, 40 aa, Artificial
  Note  Artificial = synthetic construct
TQEFWDNLEKETEGLRQEAKPVLESFKVSFLSALEEYTKK

SEQ ID NO: 46 - protein, 39 aa, Artificial
  Note  Artificial = synthetic construct
TQEFWDNLEKETEGLRQEAKPLESFKVSFLSALEEYTKK

SEQ ID NO: 47 - protein, 39 aa, Artificial
  Note  Artificial = synthetic construct
TQEFWDNLEKETEGLRQEKPVLESFKVSFLSALEEYTKK

SEQ ID NO: 48 - protein, 38 aa, Artificial
  Note  Artificial = synthetic construct
TQEFWDNLEKETEGLRQEKPLESFKVSFLSALEEYTKK

SEQ ID NO: 49 - protein, 41 aa, Artificial
  Note  Artificial = synthetic construct
LAEYHAKATEHLSTLSEKAKPVLLESFKVSFLSALEEYTKK

SEQ ID NO: 50 - protein, 41 aa, Artificial
  Note  Artificial = synthetic construct
LAEYHAKATEHLSTLSEKLVPKALESFKVSFLSALEEYTKK

SEQ ID NO: 51 - protein, 40 aa, Artificial
  Note  Artificial = synthetic construct
LAEYHAKATEHLSTLSEKKPVLLESFKVSFLSALEEYTKK

SEQ ID NO: 52 - protein, 39 aa, Artificial
  Note  Artificial = synthetic construct
LAEYHAKATEHLSTLSEKPVLLESFKVSFLSALEEYTKK

SEQ ID NO: 53 - protein, 38 aa, Artificial
  Note  Artificial = synthetic construct
LAEYHAKATEHLSTLSEKPVLESFKVSFLSALEEYTKK

SEQ ID NO: 54 - protein, 40 aa, Artificial
  Note  Artificial = synthetic construct
LAEYHAKATEHLSTLSEKAKPVLESFKVSFLSALEEYTKK

SEQ ID NO: 55 - protein, 39 aa, Artificial
  Note  Artificial = synthetic construct
LAEYHAKATEHLSTLSEKAKPLESFKVSFLSALEEYTKK

SEQ ID NO: 56 - protein, 39 aa, Artificial
  Note  Artificial = synthetic construct
LAEYHAKATEHLSTLSEKKPVLESFKVSFLSALEEYTKK

SEQ ID NO: 57 - protein, 38 aa, Artificial
  Note  Artificial = synthetic construct
LAEYHAKATEHLSTLSEKKPLESFKVSFLSALEEYTKK

SEQ ID NO: 58 - protein, 41 aa, Artificial
  Note  Artificial = synthetic construct
TQEFWDNLEKETEGLRQEKLSPLLESFKVSFLSALEEYTKK

SEQ ID NO: 59 - protein, 37 aa, Artificial
  Note  Artificial = synthetic construct
TQEFWDNLEKETEGLRQEPLESFKVSFLSALEEYTKK

SEQ ID NO: 60 - protein, 40 aa, Artificial
  Note  Artificial = synthetic construct
TQEFWDNLEKETEGLRQELSPLLESFKVSFLSALEEYTKK

SEQ ID NO: 61 - protein, 39 aa, Artificial
  Note  Artificial = synthetic construct
TQEFWDNLEKETEGLRQESPLLESFKVSFLSALEEYTKK

SEQ ID NO: 62 - protein, 41 aa, Artificial
  Note  Artificial = synthetic construct
TQEFWDNLEKETEGLRQELPSLKLESFKVSFLSALEEYTKK

SEQ ID NO: 63 - protein, 40 aa, Artificial
  Note  Artificial = synthetic construct
TQEFWDNLEKETEGLRQELPSLLESFKVSFLSALEEYTKK

SEQ ID NO: 64 - protein, 39 aa, Artificial
  Note  Artificial = synthetic construct
TQEFWDNLEKETEGLRQELPSLESFKVSFLSALEEYTKK

SEQ ID NO: 65 - protein, 41 aa, Artificial
  Note  Artificial = synthetic construct
LAEYHAKATEHLSTLSEKKLSPLLESFKVSFLSALEEYTKK

SEQ ID NO: 66 - protein, 37 aa, Artificial
  Note  Artificial = synthetic construct
LAEYHAKATEHLSTLSEKPLESFKVSFLSALEEYTKK

SEQ ID NO: 67 - protein, 40 aa, Artificial
  Note  Artificial = synthetic construct
LAEYHAKATEHLSTLSEKLSPLLESFKVSFLSALEEYTKK

SEQ ID NO: 68 - protein, 39 aa, Artificial
  Note  Artificial = synthetic construct
LAEYHAKATEHLSTLSEKSPLLESFKVSFLSALEEYTKK

SEQ ID NO: 69 - protein, 41 aa, Artificial
  Note  Artificial = synthetic construct
LAEYHAKATEHLSTLSEKLPSLKLESFKVSFLSALEEYTKK

SEQ ID NO: 70 - protein, 40 aa, Artificial
  Note  Artificial = synthetic construct
LAEYHAKATEHLSTLSEKLPSLLESFKVSFLSALEEYTKK

SEQ ID NO: 71 - protein, 39 aa, Artificial
  Note  Artificial = synthetic construct
LAEYHAKATEHLSTLSEKLPSLESFKVSFLSALEEYTKK

SEQ ID NO: 72 - protein, 41 aa, Artificial
  Note  Artificial = synthetic construct
GEEMRDRARAHVDALRTHAKPVLLESFKVSFLSALEEYTKK

SEQ ID NO: 73 - protein, 41 aa, Artificial
  Note  Artificial = synthetic construct
GEEMRDRARAHVDALRTHLVPKALESFKVSFLSALEEYTKK

SEQ ID NO: 74 - protein, 40 aa, Artificial
  Note  Artificial = synthetic construct
GEEMRDRARAHVDALRTHKPVLLESFKVSFLSALEEYTKK

SEQ ID NO: 75 - protein, 39 aa, Artificial
  Note  Artificial = synthetic construct
GEEMRDRARAHVDALRTHPVLLESFKVSFLSALEEYTKK

SEQ ID NO: 76 - protein, 38 aa, Artificial
  Note  Artificial = synthetic construct
GEEMRDRARAHVDALRTHPVLESFKVSFLSALEEYTKK

SEQ ID NO: 77 - protein, 40 aa, Artificial
  Note  Artificial = synthetic construct
GEEMRDRARAHVDALRTHAKPVLESFKVSFLSALEEYTKK

SEQ ID NO: 78 - protein, 39 aa, Artificial
  Note  Artificial = synthetic construct
GEEMRDRARAHVDALRTHAKPLESFKVSFLSALEEYTKK

SEQ ID NO: 79 - protein, 38 aa, Artificial
  Note  Artificial = synthetic construct
GEEMRDRARAHVDALRTHKPLESFKVSFLSALEEYTKK

SEQ ID NO: 80 - protein, 41 aa, Artificial
  Note  Artificial = synthetic construct
SDELRQRLAARLEALKENAKPVLLESFKVSFLSALEEYTKK

SEQ ID NO: 81 - protein, 41 aa, Artificial
  Note  Artificial = synthetic construct
SDELRQRLAARLEALKENLVPKALESFKVSFLSALEEYTKK

SEQ ID NO: 82 - protein, 40 aa, Artificial
  Note  Artificial = synthetic construct
SDELRQRLAARLEALKENKPVLLESFKVSFLSALEEYTKK

SEQ ID NO: 83 - protein, 38 aa, Artificial
  Note  Artificial = synthetic construct
DELRQRLAARLEALKENPVLLESFKVSFLSALEEYTKK

SEQ ID NO: 84 - protein, 38 aa, Artificial
  Note  Artificial = synthetic construct
SDELRQRLAARLEALKENPVLESFKVSFLSALEEYTKK

SEQ ID NO: 85 - protein, 40 aa, Artificial
  Note  Artificial = synthetic construct
SDELRQRLAARLEALKENAKPVLESFKVSFLSALEEYTKK

SEQ ID NO: 86 - protein, 39 aa, Artificial
  Note  Artificial = synthetic construct
SDELRQRLAARLEALKENAKPLESFKVSFLSALEEYTKK

SEQ ID NO: 87 - protein, 39 aa, Artificial
  Note  Artificial = synthetic construct
SDELRQRLAARLEALKENKPVLESFKVSFLSALEEYTKK

SEQ ID NO: 88 - protein, 38 aa, Artificial
  Note  Artificial = synthetic construct
SDELRQRLAARLEALKENKPLESFKVSFLSALEEYTKK

SEQ ID NO: 89 - protein, 41 aa, Artificial
  Note  Artificial = synthetic construct
LESAKVSALSALEEATKKAKPVLLESFKVSFLSALEEYTKK

SEQ ID NO: 90 - protein, 40 aa, Artificial
  Note  Artificial = synthetic construct
LESAKVSALSALEEATKKKPVLLESFKVSFLSALEEYTKK

SEQ ID NO: 91 - protein, 39 aa, Artificial
  Note  Artificial = synthetic construct
LESAKVSALSALEEATKKPVLLESFKVSFLSALEEYTKK

SEQ ID NO: 92 - protein, 38 aa, Artificial
  Note  Artificial = synthetic construct
LESAKVSALSALEEATKKPVLESFKVSFLSALEEYTKK

SEQ ID NO: 93 - protein, 40 aa, Artificial
  Note  Artificial = synthetic construct
LESAKVSALSALEEATKKAKPVLESFKVSFLSALEEYTKK

SEQ ID NO: 94 - protein, 39 aa, Artificial
  Note  Artificial = synthetic construct
LESAKVSALSALEEATKKAKPLESFKVSFLSALEEYTKK

SEQ ID NO: 95 - protein, 39 aa, Artificial
  Note  Artificial = synthetic construct
LESAKVSALSALEEATKKKPVLESFKVSFLSALEEYTKK

SEQ ID NO: 96 - protein, 38 aa, Artificial
  Note  Artificial = synthetic construct
LESAKVSALSALEEATKKKPLESFKVSFLSALEEYTKK

SEQ ID NO: 97 - protein, 41 aa, Artificial
  Note  Artificial = synthetic construct
LESAKVSALSALEEATKKAKPVLKKTYEELASLFSVKFSEL

SEQ ID NO: 98 - protein, 40 aa, Artificial
  Note  Artificial = synthetic construct
LESAKVSALSALEEATKKKPVLKKTYEELASLFSVKFSEL

SEQ ID NO: 99 - protein, 39 aa, Artificial
  Note  Artificial = synthetic construct
LESAKVSALSALEEATKKPVLKKTYEELASLFSVKFSEL

SEQ ID NO: 100 - protein, 38 aa, Artificial
  Note  Artificial = synthetic construct
LESAKVSALSALEEATKKPVKKTYEELASLFSVKFSEL

SEQ ID NO: 101 - protein, 40 aa, Artificial
  Note  Artificial = synthetic construct
LESAKVSALSALEEATKKAKPVKKTYEELASLFSVKFSEL

SEQ ID NO: 102 - protein, 39 aa, Artificial
  Note  Artificial = synthetic construct
LESAKVSALSALEEATKKAKPKKTYEELASLFSVKFSEL

SEQ ID NO: 103 - protein, 39 aa, Artificial
  Note  Artificial = synthetic construct
LESAKVSALSALEEATKKKPVKKTYEELASLFSVKFSEL

SEQ ID NO: 104 - protein, 38 aa, Artificial
  Note  Artificial = synthetic construct
LESAKVSALSALEEATKKKPKKTYEELASLFSVKFSEL

SEQ ID NO: 105 - protein, 41 aa, Artificial
  Note  Artificial = synthetic construct
KKTAEELASLASVKASELAKPVLKKTYEELASLFSVKFSEL

SEQ ID NO: 106 - protein, 41 aa, Artificial
  Note  Artificial = synthetic construct
KKTAEELASLASVKASELAKPVLLESFKVSFLSALEEYTKK

SEQ ID NO: 107 - protein, 41 aa, Artificial
  Note  Artificial = synthetic construct
LESAKVSALSALEEATKKLVPKALESFKVSFLSALEEYTKK

SEQ ID NO: 108 - protein, 41 aa, Artificial
  Note  Artificial = synthetic construct
LESAKVSALSALEEATKKLVPKAKKTYEELASLFSVKFSEL

SEQ ID NO: 109 - protein, 41 aa, Artificial
  Note  Artificial = synthetic construct
KKTAEELASLASVKASELLVPKAKKTYEELASLFSVKFSEL

SEQ ID NO: 110 - protein, 41 aa, Artificial
  Note  Artificial = synthetic construct
KKTAEELASLASVKASELLVPKALESFKVSFLSALEEYTKK

SEQ ID NO: 111 - protein, 64 aa, Artificial
  Note  Artificial = synthetic construct
PRGGSVLVTPKATQEFWDNLEKETEGLRQEAKPVLLESFKVSFLSALEEYTKKAKPVLWRWWWW

SEQ ID NO: 112 - protein, 53 aa, Artificial
  Note  Artificial = synthetic construct
PRGGSVLVTPKATQEFWDNLEKETEGLRQEAKPVLLESFKVSFLSALEEYTKK

SEQ ID NO: 113 - protein, 52 aa, Artificial
  Note  Artificial = synthetic construct
TQEFWDNLEKETEGLRQEAKPVLLESFKVSFLSALEEYTKKAKPVLWRWWWW

SEQ ID NO: 114 - protein, 64 aa, Artificial
  Note  Artificial = synthetic construct
PRGGSVLVTPKALAEYHAKATEHLSTLSEKAKPVLLESFKVSFLSALEEYTKKAKPVLWRWWWW

SEQ ID NO: 115 - protein, 53 aa, Artificial
  Note  Artificial = synthetic construct
PRGGSVLVTPKALAEYHAKATEHLSTLSEKAKPVLLESFKVSFLSALEEYTKK

SEQ ID NO: 116 - protein, 52 aa, Artificial
  Note  Artificial = synthetic construct
LAEYHAKATEHLSTLSEKAKPVLLESFKVSFLSALEEYTKKAKPVLWRWWWW

SEQ ID NO: 117 - protein, 66 aa, Artificial
  Note  Artificial = synthetic construct
PRGGSVLVTLPSLKTQEFWDNLEKETEGLRQEKLSPLLESFKVSFLSALEEYTKKALSPLWRWWWW

SEQ ID NO: 118 - protein, 55 aa, Artificial
  Note  Artificial = synthetic construct
PRGGSVLVTLPSLKTQEFWDNLEKETEGLRQEKLSPLLESFKVSFLSALEEYTKK

SEQ ID NO: 119 - protein, 52 aa, Artificial
  Note  Artificial = synthetic construct
TQEFWDNLEKETEGLRQEKLSPLLESFKVSFLSALEEYTKKALSPLWRWWWW

SEQ ID NO: 120 - protein, 66 aa, Artificial
  Note  Artificial = synthetic construct
PRGGSVLVTLPSLKLAEYHAKATEHLSTLSEKKLSPLLESFKVSFLSALEEYTKKALSPLWRWWWW

SEQ ID NO: 121 - protein, 55 aa, Artificial
  Note  Artificial = synthetic construct
PRGGSVLVTLPSLKLAEYHAKATEHLSTLSEKKLSPLLESFKVSFLSALEEYTKK

SEQ ID NO: 122 - protein, 52 aa, Artificial
  Note  Artificial = synthetic construct
LAEYHAKATEHLSTLSEKKLSPLLESFKVSFLSALEEYTKKALSPLWRWWWW

SEQ ID NO: 123 - protein, 64 aa, Artificial
  Note  Artificial = synthetic construct
PRGGSVLVTPKAGEEMRDRARAHVDALRTHAKPVLLESFKVSFLSALEEYTKKAKPVLWRWWWW

SEQ ID NO: 124 - protein, 53 aa, Artificial
  Note  Artificial = synthetic construct
PRGGSVLVTPKAGEEMRDRARAHVDALRTHAKPVLLESFKVSFLSALEEYTKK

SEQ ID NO: 125 - protein, 52 aa, Artificial
  Note  Artificial = synthetic construct
GEEMRDRARAHVDALRTHAKPVLLESFKVSFLSALEEYTKKAKPVLWRWWWW

SEQ ID NO: 126 - protein, 64 aa, Artificial
  Note  Artificial = synthetic construct
PRGGSVLVTPKASDELRQRLAARLEALKENAKPVLLESFKVSFLSALEEYTKKAKPVLWRWWWW

SEQ ID NO: 127 - protein, 53 aa, Artificial
  Note  Artificial = synthetic construct
PRGGSVLVTPKASDELRQRLAARLEALKENAKPVLLESFKVSFLSALEEYTKK

SEQ ID NO: 128 - protein, 52 aa, Artificial
  Note  Artificial = synthetic construct
SDELRQRLAARLEALKENAKPVLLESFKVSFLSALEEYTKKAKPVLWRWWWW

SEQ ID NO: 129 - protein, 64 aa, Artificial
  Note  Artificial = synthetic construct
PRGGSVLVTPKALESAKVSALSALEEATKKAKPVLLESFKVSFLSALEEYTKKAKPVLWRWWWW

SEQ ID NO: 130 - protein, 53 aa, Artificial
  Note  Artificial = synthetic construct
PRGGSVLVTPKALESAKVSALSALEEATKKAKPVLLESFKVSFLSALEEYTKK

SEQ ID NO: 131 - protein, 52 aa, Artificial
  Note  Artificial = synthetic construct
LESAKVSALSALEEATKKAKPVLLESFKVSFLSALEEYTKKAKPVLWRWWWW

SEQ ID NO: 132 - protein, 64 aa, Artificial
  Note  Artificial = synthetic construct
PRGGSVLVTPKAKKTAEELASLASVKASELAKPVLLESFKVSFLSALEEYTKKAKPVLWRWWWW

SEQ ID NO: 133 - protein, 53 aa, Artificial
  Note  Artificial = synthetic construct
PRGGSVLVTPKAKKTAEELASLASVKASELAKPVLLESFKVSFLSALEEYTKK

SEQ ID NO: 134 - protein, 52 aa, Artificial
  Note  Artificial = synthetic construct
KKTAEELASLASVKASELAKPVLLESFKVSFLSALEEYTKKAKPVLWRWWWW

SEQ ID NO: 135 - protein, 64 aa, Artificial
  Note  Artificial = synthetic construct
PRGGSVLVTPKAKKTAEELASLASVKASELAKPVLKKTYEELASLFSVKFSELAKPVLWRWWWW

SEQ ID NO: 136 - protein, 53 aa, Artificial
  Note  Artificial = synthetic construct
PRGGSVLVTPKAKKTAEELASLASVKASELAKPVLKKTYEELASLFSVKFSEL

SEQ ID NO: 137 - protein, 47 aa, Artificial
  Note  Artificial = synthetic construct
KKTAEELASLASVKASELAKPVLKKTYEELASLFSVKFSELWRWWWW

SEQ ID NO: 138 - protein, 66 aa, Artificial
  Note  Artificial = synthetic construct
PRGGSVLVTLPSLKLESAKVSALSALEEATKKAKPVLLESFKVSFLSALEEYTKKALSPLWRWWWW

SEQ ID NO: 139 - protein, 55 aa, Artificial
  Note  Artificial = synthetic construct
PRGGSVLVTLPSLKLESAKVSALSALEEATKKAKPVLLESFKVSFLSALEEYTKK

SEQ ID NO: 140 - protein, 52 aa, Artificial
  Note  Artificial = synthetic construct
LESAKVSALSALEEATKKAKPVLLESFKVSFLSALEEYTKKALSPLWRWWWW

SEQ ID NO: 141 - protein, 66 aa, Artificial
  Note  Artificial = synthetic construct
PRGGSVLVTLPSLKKKTAEELASLASVKASELAKPVLLESFKVSFLSALEEYTKKALSPLWRWWWW

SEQ ID NO: 142 - protein, 55 aa, Artificial
  Note  Artificial = synthetic construct
PRGGSVLVTLPSLKKKTAEELASLASVKASELAKPVLLESFKVSFLSALEEYTKK

SEQ ID NO: 143 - protein, 52 aa, Artificial
  Note  Artificial = synthetic construct
KKTAEELASLASVKASELAKPVLLESFKVSFLSALEEYTKKALSPLWRWWWW

SEQ ID NO: 144 - protein, 66 aa, Artificial
  Note  Artificial = synthetic construct
PRGGSVLVTLPSLKKKTAEELASLASVKASELAKPVLKKTYEELASLFSVKFSELALSPLWRWWWW

SEQ ID NO: 145 - protein, 55 aa, Artificial
  Note  Artificial = synthetic construct
PRGGSVLVTLPSLKKKTAEELASLASVKASELAKPVLKKTYEELASLFSVKFSEL

SEQ ID NO: 146 - protein, 62 aa, Artificial
  Note  Artificial = synthetic construct
PRGGSVLVTLPSLKKKTAEELASLASVKASELPLESFKVSFLSALEEYTKKALSPLWRWWWW

SEQ ID NO: 147 - protein, 51 aa, Artificial
  Note  Artificial = synthetic construct
PRGGSVLVTLPSLKKKTAEELASLASVKASELPLESFKVSFLSALEEYTKK

SEQ ID NO: 148 - protein, 48 aa, Artificial
  Note  Artificial = synthetic construct
KKTAEELASLASVKASELPLESFKVSFLSALEEYTKKALSPLWRWWWW

SEQ ID NO: 149 - protein, 62 aa, Artificial
  Note  Artificial = synthetic construct
PRGGSVLVTLPSLKKKTAEELASLASVKASELKPKTYEELASLFSVKFSELALSPLWRWWWW

SEQ ID NO: 150 - protein, 51 aa, Artificial
  Note  Artificial = synthetic construct
PRGGSVLVTLPSLKKKTAEELASLASVKASELPKKTYEELASLFSVKFSEL

SEQ ID NO: 151 - protein, 43 aa, Artificial
  Note  Artificial = synthetic construct
KKTAEELASLASVKASELPKKTYEELASLFSVKFSELWRWWWW

SEQ ID NO: 152 - protein, 62 aa, Artificial
  Note  Artificial = synthetic construct
PRGGSVLVTLPSLKKKTAEELASLASVKASELPKKTYEELASLFSVKFSELALSPLWRWWWW

SEQ ID NO: 153 - protein, 50 aa, Artificial
  Note  Artificial = synthetic construct
PRGGSVLVTLESAKVSALSALEEATKKAKPVLLESFKVSFLSALEEYTKK

SEQ ID NO: 154 - protein, 50 aa, Artificial; identical to SEQ ID NO: 153
  Note  Artificial = synthetic construct

SEQ ID NO: 155 - protein, 50 aa, Artificial; identical to SEQ ID NO: 153
  Note  Artificial = synthetic construct

SEQ ID NO: 156 - protein, 50 aa, Artificial; identical to SEQ ID NO: 153
  Note  Artificial = synthetic construct

SEQ ID NO: 157 - protein, 50 aa, Artificial; identical to SEQ ID NO: 153
  Note  Artificial = synthetic construct

SEQ ID NO: 158 - protein, 50 aa, Artificial; identical to SEQ ID NO: 153
  Note  Artificial = synthetic construct

SEQ ID NO: 159 - protein, 50 aa, Artificial
  Note  Artificial = synthetic construct
TVLVSGGRPLESAKVSALSALEEATKKAKPVLLESFKVSFLSALEEYTKK

SEQ ID NO: 160 - protein, 50 aa, Artificial; identical to SEQ ID NO: 159
  Note  Artificial = synthetic construct

SEQ ID NO: 161 - protein, 50 aa, Artificial; identical to SEQ ID NO: 159
  Note  Artificial = synthetic construct

SEQ ID NO: 162 - protein, 50 aa, Artificial; identical to SEQ ID NO: 159
  Note  Artificial = synthetic construct

SEQ ID NO: 163 - protein, 50 aa, Artificial; identical to SEQ ID NO: 159
  Note  Artificial = synthetic construct

SEQ ID NO: 164 - protein, 50 aa, Artificial; identical to SEQ ID NO: 159
  Note  Artificial = synthetic construct

SEQ ID NO: 165 - protein, 53 aa, Artificial; identical to SEQ ID NO: 130
  Note  Artificial = synthetic construct

SEQ ID NO: 166 - protein, 53 aa, Artificial; identical to SEQ ID NO: 130
  Note  Artificial = synthetic construct

SEQ ID NO: 167 - protein, 53 aa, Artificial; identical to SEQ ID NO: 130
  Note  Artificial = synthetic construct

SEQ ID NO: 168 - protein, 53 aa, Artificial; identical to SEQ ID NO: 130
  Note  Artificial = synthetic construct

SEQ ID NO: 169 - protein, 53 aa, Artificial; identical to SEQ ID NO: 130
  Note  Artificial = synthetic construct

SEQ ID NO: 170 - protein, 53 aa, Artificial; identical to SEQ ID NO: 130
  Note  Artificial = synthetic construct

SEQ ID NO: 171 - protein, 53 aa, Artificial
  Note  Artificial = synthetic construct
TVLVSGGRPPKALESAKVSALSALEEATKKAKPVLLESFKVSFLSALEEYTKK

SEQ ID NO: 172 - protein, 53 aa, Artificial; identical to SEQ ID NO: 171
  Note  Artificial = synthetic construct

SEQ ID NO: 173 - protein, 53 aa, Artificial; identical to SEQ ID NO: 171
  Note  Artificial = synthetic construct

SEQ ID NO: 174 - protein, 53 aa, Artificial; identical to SEQ ID NO: 171
  Note  Artificial = synthetic construct

SEQ ID NO: 175 - protein, 53 aa, Artificial; identical to SEQ ID NO: 171
  Note  Artificial = synthetic construct

SEQ ID NO: 176 - protein, 53 aa, Artificial; identical to SEQ ID NO: 171
  Note  Artificial = synthetic construct

SEQ ID NO: 177 - protein, 64 aa, Artificial
  Note  Artificial = synthetic construct
WRWWWWPKATQEFWDNLEKETEGLRQEAKPVLLESFKVSFLSALEEYTKKAKPVLPRGGSVLVT

SEQ ID NO: 178 - protein, 55 aa, Artificial
  Note  Artificial = synthetic construct
TQEFWDNLEKETEGLRQEAKPVLLESFKVSFLSALEEYTKKAKPVLPRGGSVLVT

SEQ ID NO: 179 - protein, 50 aa, Artificial
  Note  Artificial = synthetic construct
WRWWWWPKATQEFWDNLEKETEGLRQEAKPVLLESFKVSFLSALEEYTKK

SEQ ID NO: 180 - protein, 64 aa, Artificial
  Note  Artificial = synthetic construct
WRWWWWPKALAEYHAKATEHLSTLSEKAKPVLLESFKVSFLSALEEYTKKAKPVLPRGGSVLVT

SEQ ID NO: 181 - protein, 55 aa, Artificial
  Note  Artificial = synthetic construct
LAEYHAKATEHLSTLSEKAKPVLLESFKVSFLSALEEYTKKAKPVLPRGGSVLVT

SEQ ID NO: 182 - protein, 50 aa, Artificial
  Note  Artificial = synthetic construct
WRWWWWPKALAEYHAKATEHLSTLSEKAKPVLLESFKVSFLSALEEYTKK

SEQ ID NO: 183 - protein, 66 aa, Artificial
  Note  Artificial = synthetic construct
WRWWWWLPSLKTQEFWDNLEKETEGLRQEKLSPLLESFKVSFLSALEEYTKKALSPLPRGGSVLVT

SEQ ID NO: 184 - protein, 55 aa, Artificial
  Note  Artificial = synthetic construct
TQEFWDNLEKETEGLRQEKLSPLLESFKVSFLSALEEYTKKALSPLPRGGSVLVT

SEQ ID NO: 185 - protein, 52 aa, Artificial
  Note  Artificial = synthetic construct
WRWWWWLPSLKTQEFWDNLEKETEGLRQEKLSPLLESFKVSFLSALEEYTKK

SEQ ID NO: 186 - protein, 66 aa, Artificial
  Note  Artificial = synthetic construct
WRWWWWLPSLKLAEYHAKATEHLSTLSEKKLSPLLESFKVSFLSALEEYTKKALSPLPRGGSVLVT

SEQ ID NO: 187 - protein, 55 aa, Artificial
  Note  Artificial = synthetic construct
LAEYHAKATEHLSTLSEKKLSPLLESFKVSFLSALEEYTKKALSPLPRGGSVLVT

SEQ ID NO: 188 - protein, 52 aa, Artificial
  Note  Artificial = synthetic construct
WRWWWWLPSLKLAEYHAKATEHLSTLSEKKLSPLLESFKVSFLSALEEYTKK

SEQ ID NO: 189 - protein, 64 aa, Artificial
  Note  Artificial = synthetic construct
WRWWWWPKAGEEMRDRARAHVDALRTHAKPVLLESFKVSFLSALEEYTKKAKPVLPRGGSVLVT

SEQ ID NO: 190 - protein, 55 aa, Artificial
  Note  Artificial = synthetic construct
GEEMRDRARAHVDALRTHAKPVLLESFKVSFLSALEEYTKKAKPVLPRGGSVLVT

SEQ ID NO: 191 - protein, 50 aa, Artificial
  Note  Artificial = synthetic construct
WRWWWWPKAGEEMRDRARAHVDALRTHAKPVLLESFKVSFLSALEEYTKK

SEQ ID NO: 192 - protein, 64 aa, Artificial
  Note  Artificial = synthetic construct
WRWWWWPKASDELRQRLAARLEALKENAKPVLLESFKVSFLSALEEYTKKAKPVLPRGGSVLVT

SEQ ID NO: 193 - protein, 55 aa, Artificial
  Note  Artificial = synthetic construct
SDELRQRLAARLEALKENAKPVLLESFKVSFLSALEEYTKKAKPVLPRGGSVLVT

SEQ ID NO: 194 - protein, 50 aa, Artificial
  Note  Artificial = synthetic construct
WRWWWWPKASDELRQRLAARLEALKENAKPVLLESFKVSFLSALEEYTKK

SEQ ID NO: 195 - protein, 66 aa, Artificial
  Note  Artificial = synthetic construct
WRWWWWLPSLKGEEMRDRARAHVDALRTHAKPVLLESFKVSFLSALEEYTKKALSPLPRGGSVLVT

SEQ ID NO: 196 - protein, 55 aa, Artificial
  Note  Artificial = synthetic construct
GEEMRDRARAHVDALRTHAKPVLLESFKVSFLSALEEYTKKALSPLPRGGSVLVT

SEQ ID NO: 197 - protein, 52 aa, Artificial
  Note  Artificial = synthetic construct
WRWWWWLPSLKGEEMRDRARAHVDALRTHAKPVLLESFKVSFLSALEEYTKK

SEQ ID NO: 198 - protein, 66 aa, Artificial
  Note  Artificial = synthetic construct
WRWWWWLPSLKSDELRQRLAARLEALKENAKPVLLESFKVSFLSALEEYTKKALSPLPRGGSVLVT

SEQ ID NO: 199 - protein, 55 aa, Artificial
  Note  Artificial = synthetic construct
SDELRQRLAARLEALKENAKPVLLESFKVSFLSALEEYTKKALSPLPRGGSVLVT

SEQ ID NO: 200 - protein, 52 aa, Artificial
  Note  Artificial = synthetic construct
WRWWWWLPSLKSDELRQRLAARLEALKENAKPVLLESFKVSFLSALEEYTKK

SEQ ID NO: 201 - protein, 64 aa, Artificial
  Note  Artificial = synthetic construct
WRWWWWPKALESAKVSALSALEEATKKAKPVLLESFKVSFLSALEEYTKKAKPVLPRGGSVLVT

SEQ ID NO: 202 - protein, 55 aa, Artificial
  Note  Artificial = synthetic construct
LESAKVSALSALEEATKKAKPVLLESFKVSFLSALEEYTKKAKPVLPRGGSVLVT

SEQ ID NO: 203 - protein, 50 aa, Artificial
  Note  Artificial = synthetic construct
WRWWWWPKALESAKVSALSALEEATKKAKPVLLESFKVSFLSALEEYTKK

SEQ ID NO: 204 - protein, 64 aa, Artificial
  Note  Artificial = synthetic construct
WRWWWWPKAKKTAEELASLASVKASELAKPVLLESFKVSFLSALEEYTKKAKPVLPRGGSVLVT

SEQ ID NO: 205 - protein, 55 aa, Artificial
  Note  Artificial = synthetic construct
KKTAEELASLASVKASELAKPVLLESFKVSFLSALEEYTKKAKPVLPRGGSVLVT

SEQ ID NO: 206 - protein, 50 aa, Artificial
  Note  Artificial = synthetic construct
WRWWWWPKAKKTAEELASLASVKASELAKPVLLESFKVSFLSALEEYTKK

SEQ ID NO: 207 - protein, 64 aa, Artificial
  Note  Artificial = synthetic construct
WRWWWWPKAKKTAEELASLASVKASELAKPVLKKTYEELASLFSVKFSELAKPVLPRGGSVLVT

SEQ ID NO: 208 - protein, 55 aa, Artificial
  Note  Artificial = synthetic construct
KKTAEELASLASVKASELAKPVLKKTYEELASLFSVKFSELAKPVLPRGGSVLVT

SEQ ID NO: 209 - protein, 50 aa, Artificial
  Note  Artificial = synthetic construct
WRWWWWPKAKKTAEELASLASVKASELAKPVLKKTYEELASLFSVKFSEL

SEQ ID NO: 210 - protein, 66 aa, Artificial
  Note  Artificial = synthetic construct
WRWWWWLPSLKLESAKVSALSALEEATKKAKPVLLESFKVSFLSALEEYTKKALSPLPRGGSVLVT

SEQ ID NO: 211 - protein, 55 aa, Artificial
  Note  Artificial = synthetic construct
LESAKVSALSALEEATKKAKPVLLESFKVSFLSALEEYTKKALSPLPRGGSVLVT

SEQ ID NO: 212 - protein, 52 aa, Artificial
  Note  Artificial = synthetic construct
WRWWWWLPSLKLESAKVSALSALEEATKKAKPVLLESFKVSFLSALEEYTKK

SEQ ID NO: 213 - protein, 66 aa, Artificial
  Note  Artificial = synthetic construct
WRWWWWLPSLKKKTAEELASLASVKASELAKPVLLESFKVSFLSALEEYTKKALSPLPRGGSVLVT

SEQ ID NO: 214 - protein, 55 aa, Artificial
  Note  Artificial = synthetic construct
KKTAEELASLASVKASELAKPVLLESFKVSFLSALEEYTKKALSPLPRGGSVLVT

SEQ ID NO: 215 - protein, 52 aa, Artificial
  Note  Artificial = synthetic construct
WRWWWWLPSLKKKTAEELASLASVKASELAKPVLLESFKVSFLSALEEYTKK

SEQ ID NO: 216 - protein, 66 aa, Artificial
  Note  Artificial = synthetic construct
WRWWWWLPSLKKKTAEELASLASVKASELAKPVLKKTYEELASLFSVKFSELALSPLPRGGSVLVT

SEQ ID NO: 217 - protein, 55 aa, Artificial
  Note  Artificial = synthetic construct
KKTAEELASLASVKASELAKPVLKKTYEELASLFSVKFSELALSPLPRGGSVLVT

SEQ ID NO: 218 - protein, 52 aa, Artificial
  Note  Artificial = synthetic construct
WRWWWWLPSLKKKTAEELASLASVKASELAKPVLKKTYEELASLFSVKFSEL

SEQ ID NO: 219 - protein, 62 aa, Artificial
  Note  Artificial = synthetic construct
WRWWWWLPSLKKKTAEELASLASVKASELPLESFKVSFLSALEEYTKKALSPLPRGGSVLVT

SEQ ID NO: 220 - protein, 51 aa, Artificial
  Note  Artificial = synthetic construct
KKTAEELASLASVKASELPLESFKVSFLSALEEYTKKALSPLPRGGSVLVT

SEQ ID NO: 221 - protein, 48 aa, Artificial
  Note  Artificial = synthetic construct
WRWWWWLPSLKKKTAEELASLASVKASELPLESFKVSFLSALEEYTKK

SEQ ID NO: 222 - protein, 62 aa, Artificial
  Note  Artificial = synthetic construct
WRWWWWLPSLKKKTAEELASLASVKASELPKKTYEELASLFSVKFSELALSPLPRGGSVLVT

SEQ ID NO: 223 - protein, 51 aa, Artificial
  Note  Artificial = synthetic construct
KKTAEELASLASVKASELPKKTYEELASLFSVKFSELALSPLPRGGSVLVT

SEQ ID NO: 224 - protein, 48 aa, Artificial
  Note  Artificial = synthetic construct
WRWWWWLPSLKKKTAEELASLASVKASELPKKTYEELASLFSVKFSEL

SEQ ID NO: 225 - protein, 48 aa, Artificial
  Note  Artificial = synthetic construct
SPLTQEFWDNLEKETEGLRQEAKPVLLESFKVSFLSALEEYTKKLNTQ

SEQ ID NO: 226 - protein, 45 aa, Artificial
  Note  Artificial = synthetic construct
TQEFWDNLEKETEGLRQEAKPVLLESFKVSFLSALEEYTKKLNTQ

SEQ ID NO: 227 - protein, 44 aa, Artificial
  Note  Artificial = synthetic construct
SPLTQEFWDNLEKETEGLRQEAKPVLLESFKVSFLSALEEYTKK

SEQ ID NO: 228 - protein, 48 aa, Artificial
  Note  Artificial = synthetic construct
SPLLAEYHAKATEHLSTLSEKAKPVLLESFKVSFLSALEEYTKKLNTQ

SEQ ID NO: 229 - protein, 45 aa, Artificial
  Note  Artificial = synthetic construct
LAEYHAKATEHLSTLSEKAKPVLLESFKVSFLSALEEYTKKLNTQ

SEQ ID NO: 230 - protein, 44 aa, Artificial
  Note  Artificial = synthetic construct
SPLLAEYHAKATEHLSTLSEKAKPVLLESFKVSFLSALEEYTKK

SEQ ID NO: 231 - protein, 48 aa, Artificial
  Note  Artificial = synthetic construct
SPLTQEFWDNLEKETEGLRQEKLSPLLESFKVSFLSALEEYTKKLNTQ

SEQ ID NO: 232 - protein, 45 aa, Artificial
  Note  Artificial = synthetic construct
TQEFWDNLEKETEGLRQEKLSPLLESFKVSFLSALEEYTKKLNTQ

SEQ ID NO: 233 - protein, 44 aa, Artificial
  Note  Artificial = synthetic construct
SPLTQEFWDNLEKETEGLRQEKLSPLLESFKVSFLSALEEYTKK

SEQ ID NO: 234 - protein, 48 aa, Artificial
  Note  Artificial = synthetic construct
SPLLAEYHAKATEHLSTLSEKKLSPLLESFKVSFLSALEEYTKKLNTQ

SEQ ID NO: 235 - protein, 45 aa, Artificial
  Note  Artificial = synthetic construct
LAEYHAKATEHLSTLSEKKLSPLLESFKVSFLSALEEYTKKLNTQ

SEQ ID NO: 236 - protein, 44 aa, Artificial
  Note  Artificial = synthetic construct
SPLLAEYHAKATEHLSTLSEKKLSPLLESFKVSFLSALEEYTKK

SEQ ID NO: 237 - protein, 48 aa, Artificial
  Note  Artificial = synthetic construct
SPLGEEMRDRARAHVDALRTHAKPVLLESFKVSFLSALEEYTKKLNTQ

SEQ ID NO: 238 - protein, 45 aa, Artificial
  Note  Artificial = synthetic construct
GEEMRDRARAHVDALRTHAKPVLLESFKVSFLSALEEYTKKLNTQ

SEQ ID NO: 239 - protein, 44 aa, Artificial
  Note  Artificial = synthetic construct
SPLGEEMRDRARAHVDALRTHAKPVLLESFKVSFLSALEEYTKK

SEQ ID NO: 240 - protein, 48 aa, Artificial
  Note  Artificial = synthetic construct
SPLSDELRQRLAARLEALKENAKPVLLESFKVSFLSALEEYTKKLNTQ

SEQ ID NO: 241 - protein, 45 aa, Artificial
  Note  Artificial = synthetic construct
SDELRQRLAARLEALKENAKPVLLESFKVSFLSALEEYTKKLNTQ

SEQ ID NO: 242 - protein, 44 aa, Artificial
  Note  Artificial = synthetic construct
SPLSDELRQRLAARLEALKENAKPVLLESFKVSFLSALEEYTKK

SEQ ID NO: 243 - protein, 48 aa, Artificial
  Note  Artificial = synthetic construct
SPLLESAKVSALSALEEATKKAKPVLLESFKVSFLSALEEYTKKLNTQ

SEQ ID NO: 244 - protein, 45 aa, Artificial
  Note  Artificial = synthetic construct
LESAKVSALSALEEATKKAKPVLLESFKVSFLSALEEYTKKLNTQ

SEQ ID NO: 245 - protein, 44 aa, Artificial
  Note  Artificial = synthetic construct
SPLLESAKVSALSALEEATKKAKPVLLESFKVSFLSALEEYTKK

SEQ ID NO: 246 - protein, 48 aa, Artificial
  Note  Artificial = synthetic construct
SPLKKTAEELASLASVKASELAKPVLLESFKVSFLSALEEYTKKLNTQ

SEQ ID NO: 247 - protein, 45 aa, Artificial
  Note  Artificial = synthetic construct
KKTAEELASLASVKASELAKPVLLESFKVSFLSALEEYTKKLNTQ

SEQ ID NO: 248 - protein, 44 aa, Artificial
  Note  Artificial = synthetic construct
SPLKKTAEELASLASVKASELAKPVLLESFKVSFLSALEEYTKK

SEQ ID NO: 249 - protein, 48 aa, Artificial
  Note  Artificial = synthetic construct
SPLKKTAEELASLASVKASELAKPVLKKTYEELASLFSVKFSELLNTQ

SEQ ID NO: 250 - protein, 45 aa, Artificial
  Note  Artificial = synthetic construct
KKTAEELASLASVKASELAKPVLKKTYEELASLFSVKFSELLNTQ

SEQ ID NO: 251 - protein, 44 aa, Artificial
  Note  Artificial = synthetic construct
SPLKKTAEELASLASVKASELAKPVLKKTYEELASLFSVKFSEL

SEQ ID NO: 252 - protein, 48 aa, Artificial
  Note  Artificial = synthetic construct
SPLLESAKVSALSALEEATKKAKPVLKKTYEELASLFSVKFSELLNTQ

SEQ ID NO: 253 - protein, 45 aa, Artificial
  Note  Artificial = synthetic construct
LESAKVSALSALEEATKKAKPVLKKTYEELASLFSVKFSELLNTQ

SEQ ID NO: 254 - protein, 44 aa, Artificial
  Note  Artificial = synthetic construct
SPLLESAKVSALSALEEATKKAKPVLKKTYEELASLFSVKFSEL

SEQ ID NO: 255 - protein, 44 aa, Artificial
  Note  Artificial = synthetic construct
SPLKKTAEELASLASVKASELPLESFKVSFLSALEEYTKKLNTQ

SEQ ID NO: 256 - protein, 41 aa, Artificial
  Note  Artificial = synthetic construct
KKTAEELASLASVKASELPLESFKVSFLSALEEYTKKLNTQ

SEQ ID NO: 257 - protein, 40 aa, Artificial
  Note  Artificial = synthetic construct
SPLKKTAEELASLASVKASELPLESFKVSFLSALEEYTKK

SEQ ID NO: 258 - protein, 44 aa, Artificial
  Note  Artificial = synthetic construct
SPLKKTAEELASLASVKASELPKKTYEELASLFSVKFSELLNTQ

SEQ ID NO: 259 - protein, 41 aa, Artificial
  Note  Artificial = synthetic construct
KKTAEELASLASVKASELPKKTYEELASLFSVKFSELLNTQ

SEQ ID NO: 260 - protein, 40 aa, Artificial
  Note  Artificial = synthetic construct
SPLKKTAEELASLASVKASELPKKTYEELASLFSVKFSEL

SEQ ID NO: 261 - protein, 38 aa, Artificial
  Note  Artificial = synthetic construct
SPLLESAKVSALSALEEATKKPKKTYEELASLFSVKFS

SEQ ID NO: 262 - protein, 57 aa, Artificial
  Note  Artificial = synthetic construct
PRGGSVLVTPKATQEFWDNLEKETEGLRQEAKPVLLESFKVSFLSALEEYTKKLNTQ

SEQ ID NO: 263 - protein, 55 aa, Artificial
  Note  Artificial = synthetic construct
SPLTQEFWDNLEKETEGLRQEAKPVLLESFKVSFLSALEEYTKKAKPVLWRWWWW

SEQ ID NO: 264 - protein, 57 aa, Artificial
  Note  Artificial = synthetic construct
PRGGSVLVTPKALAEYHAKATEHLSTLSEKAKPVLLESFKVSFLSALEEYTKKLNTQ

SEQ ID NO: 265 - protein, 55 aa, Artificial
  Note  Artificial = synthetic construct
SPLLAEYHAKATEHLSTLSEKAKPVLLESFKVSFLSALEEYTKKAKPVLWRWWWW

SEQ ID NO: 266 - protein, 59 aa, Artificial
  Note  Artificial = synthetic construct
PRGGSVLVTLPSLKTQEFWDNLEKETEGLRQEKLSPLLESFKVSFLSALEEYTKKLNTQ

SEQ ID NO: 267 - protein, 55 aa, Artificial
  Note  Artificial = synthetic construct
SPLTQEFWDNLEKETEGLRQEKLSPLLESFKVSFLSALEEYTKKALSPLWRWWWW

SEQ ID NO: 268 - protein, 59 aa, Artificial
  Note  Artificial = synthetic construct
PRGGSVLVTLPSLKLAEYHAKATEHLSTLSEKKLSPLLESFKVSFLSALEEYTKKLNTQ

SEQ ID NO: 269 - protein, 55 aa, Artificial
  Note  Artificial = synthetic construct
SPLLAEYHAKATEHLSTLSEKKLSPLLESFKVSFLSALEEYTKKALSPLWRWWWW

SEQ ID NO: 270 - protein, 57 aa, Artificial
  Note  Artificial = synthetic construct
PRGGSVLVTPKAGEEMRDRARAHVDALRTHAKPVLLESFKVSFLSALEEYTKKLNTQ

SEQ ID NO: 271 - protein, 55 aa, Artificial
  Note  Artificial = synthetic construct
SPLGEEMRDRARAHVDALRTHAKPVLLESFKVSFLSALEEYTKKAKPVLWRWWWW

SEQ ID NO: 272 - protein, 57 aa, Artificial
  Note  Artificial = synthetic construct
PRGGSVLVTPKASDELRQRLAARLEALKENAKPVLLESFKVSFLSALEEYTKKLNTQ

SEQ ID NO: 273 - protein, 55 aa, Artificial
  Note  Artificial = synthetic construct
SPLSDELRQRLAARLEALKENAKPVLLESFKVSFLSALEEYTKKAKPVLWRWWWW

SEQ ID NO: 274 - protein, 59 aa, Artificial
  Note  Artificial = synthetic construct
PRGGSVLVTLPSLKGEEMRDRARAHVDALRTHAKPVLLESFKVSFLSALEEYTKKLNTQ

SEQ ID NO: 275 - protein, 55 aa, Artificial
  Note  Artificial = synthetic construct
SPLGEEMRDRARAHVDALRTHAKPVLLESFKVSFLSALEEYTKKALSPLWRWWWW

SEQ ID NO: 276 - protein, 59 aa, Artificial
  Note  Artificial = synthetic construct
PRGGSVLVTLPSLKSDELRQRLAARLEALKENAKPVLLESFKVSFLSALEEYTKKLNTQ

SEQ ID NO: 277 - protein, 55 aa, Artificial
  Note  Artificial = synthetic construct
SPLSDELRQRLAARLEALKENAKPVLLESFKVSFLSALEEYTKKALSPLWRWWWW

SEQ ID NO: 278 - protein, 57 aa, Artificial
  Note  Artificial = synthetic construct
PRGGSVLVTPKALESAKVSALSALEEATKKAKPVLLESFKVSFLSALEEYTKKLNTQ

SEQ ID NO: 279 - protein, 55 aa, Artificial
  Note  Artificial = synthetic construct
SPLLESAKVSALSALEEATKKAKPVLLESFKVSFLSALEEYTKKAKPVLWRWWWW

SEQ ID NO: 280 - protein, 57 aa, Artificial
  Note  Artificial = synthetic construct
PRGGSVLVTPKAKKTAEELASLASVKASELAKPVLLESFKVSFLSALEEYTKKLNTQ

SEQ ID NO: 281 - protein, 55 aa, Artificial
  Note  Artificial = synthetic construct
SPLKKTAEELASLASVKASELAKPVLLESFKVSFLSALEEYTKKAKPVLWRWWWW

SEQ ID NO: 282 - protein, 57 aa, Artificial
  Note  Artificial = synthetic construct
PRGGSVLVTPKAKKTAEELASLASVKASELAKPVLKKTYEELASLFSVKFSELLNTQ

SEQ ID NO: 283 - protein, 55 aa, Artificial
  Note  Artificial = synthetic construct
SPLKKTAEELASLASVKASELAKPVLKKTYEELASLFSVKFSELAKPVLWRWWWW

SEQ ID NO: 284 - protein, 57 aa, Artificial
  Note  Artificial = synthetic construct
PRGGSVLVTPKALESAKVSALSALEEATKKAKPVLKKTYEELASLFSVKFSELLNTQ

SEQ ID NO: 285 - protein, 55 aa, Artificial
  Note  Artificial = synthetic construct
SPLLESAKVSALSALEEATKKAKPVLKKTYEELASLFSVKFSELAKPVLWRWWWW

SEQ ID NO: 286 - protein, 59 aa, Artificial
  Note  Artificial = synthetic construct
PRGGSVLVTLPSLKLESAKVSALSALEEATKKAKPVLLESFKVSFLSALEEYTKKLNTQ

SEQ ID NO: 287 - protein, 55 aa, Artificial
  Note  Artificial = synthetic construct
SPLLESAKVSALSALEEATKKAKPVLLESFKVSFLSALEEYTKKALSPLWRWWWW

SEQ ID NO: 288 - protein, 59 aa, Artificial
  Note  Artificial = synthetic construct
PRGGSVLVTLPSLKKKTAEELASLASVKASELAKPVLLESFKVSFLSALEEYTKKLNTQ

SEQ ID NO: 289 - protein, 55 aa, Artificial
  Note  Artificial = synthetic construct
SPLKKTAEELASLASVKASELAKPVLLESFKVSFLSALEEYTKKALSPLWRWWWW

SEQ ID NO: 290 - protein, 59 aa, Artificial
  Note  Artificial = synthetic construct
PRGGSVLVTLPSLKKKTAEELASLASVKASELAKPVLKKTYEELASLFSVKFSELLNTQ

SEQ ID NO: 291 - protein, 55 aa, Artificial
  Note  Artificial = synthetic construct
SPLKKTAEELASLASVKASELAKPVLKKTYEELASLFSVKFSELALSPLWRWWWW

SEQ ID NO: 292 - protein, 59 aa, Artificial
  Note  Artificial = synthetic construct
PRGGSVLVTLPSLKLESAKVSALSALEEATKKAKPVLKKTYEELASLFSVKFSELLNTQ

SEQ ID NO: 293 - protein, 55 aa, Artificial
  Note  Artificial = synthetic construct
SPLLESAKVSALSALEEATKKAKPVLKKTYEELASLFSVKFSELALSPLWRWWWW

SEQ ID NO: 294 - protein, 55 aa, Artificial
  Note  Artificial = synthetic construct
PRGGSVLVTLPSLKKKTAEELASLASVKASELPLESFKVSFLSALEEYTKKLNTQ

SEQ ID NO: 295 - protein, 51 aa, Artificial
  Note  Artificial = synthetic construct
SPLKKTAEELASLASVKASELPLESFKVSFLSALEEYTKKALSPLWRWWWW

SEQ ID NO: 296 - protein, 55 aa, Artificial
  Note  Artificial = synthetic construct
PRGGSVLVTLPSLKKKTAEELASLASVKASELPKKTYEELASLFSVKFSELLNTQ

SEQ ID NO: 297 - protein, 51 aa, Artificial
  Note  Artificial = synthetic construct
SPLKKTAEELASLASVKASELPKKTYEELASLFSVKFSELALSPLWRWWWW

SEQ ID NO: 298 - protein, 21 aa, Artificial
  Note  Artificial = synthetic construct
PRGGSVLVTLAKPVLWRWWWW

SEQ ID NO: 299 - protein, 21 aa, Artificial
  Note  Artificial = synthetic construct
WRWWWWLAKPVLPRGGSVLVT

SEQ ID NO: 300 - protein, 9 aa, Artificial
  Note  Artificial = synthetic construct
PRGGSVLVT

SEQ ID NO: 301 - protein, 9 aa, Artificial
  Note  Artificial = synthetic construct
PRGGSVLVT

SEQ ID NO: 302 - protein, 9 aa, Artificial
  Note  Artificial = synthetic construct
PRGGSVLVT

SEQ ID NO: 303 - protein, 9 aa, Artificial
  Note  Artificial = synthetic construct
PRGGSVLVT

SEQ ID NO: 304 - protein, 9 aa, Artificial
  Note  Artificial = synthetic construct
PRGGSVLVT

SEQ ID NO: 305 - protein, 9 aa, Artificial
  Note  Artificial = synthetic construct
PRGGSVLVT

SEQ ID NO: 306 - protein, 9 aa, Artificial
  Note  Artificial = synthetic construct
TVLVSGGRP

SEQ ID NO: 307 - protein, 9 aa, Artificial
  Note  Artificial = synthetic construct
TVLVSGGRP

SEQ ID NO: 308 - protein, 9 aa, Artificial
  Note  Artificial = synthetic construct
TVLVSGGRP

SEQ ID NO: 309 - protein, 9 aa, Artificial
  Note  Artificial = synthetic construct
TVLVSGGRP

SEQ ID NO: 310 - protein, 9 aa, Artificial
  Note  Artificial = synthetic construct
TVLVSGGRP

SEQ ID NO: 311 - protein, 9 aa, Artificial
  Note  Artificial = synthetic construct
TVLVSGGRP

SEQ ID NO: 312 - protein, 6 aa, Artificial
  Note  Artificial = synthetic construct
WWRWWW

SEQ ID NO: 313 - protein, 6 aa, Artificial
  Note  Artificial = synthetic construct
WWWRWW

SEQ ID NO: 314 - protein, 6 aa, Artificial
  Note  Artificial = synthetic construct
WWWWWR

SEQ ID NO: 315 - protein, 6 aa, Artificial
  Note  Artificial = synthetic construct
RWWWWW

SEQ ID NO: 316 - protein, 12 aa, Artificial
  Note  Artificial = synthetic construct
WRWWWWWRWWWW

SEQ ID NO: 317 - protein, 18 aa, Artificial
  Note  Artificial = synthetic construct
WRWWWWWRWWWWWRWWWW

SEQ ID NO: 318 - protein, 12 aa, Artificial
  Note  Artificial = synthetic construct
WWRWWWWWRWWW

SEQ ID NO: 319 - protein, 18 aa, Artificial
  Note  Artificial = synthetic construct
WWRWWWWWRWWWWWRWWW

SEQ ID NO: 320 - protein, 12 aa, Artificial
  Note  Artificial = synthetic construct
WWWRWWWWWRWW

SEQ ID NO: 321 - protein, 18 aa, Artificial
  Note  Artificial = synthetic construct
WWWRWWWWWRWWWWWRWW

SEQ ID NO: 322 - protein, 12 aa, Artificial
  Note  Artificial = synthetic construct
WWWWRWWWWWRW

SEQ ID NO: 323 - protein, 18 aa, Artificial
  Note  Artificial = synthetic construct
WWWWRWWWWWRWWWWWRW

SEQ ID NO: 324 - protein, 12 aa, Artificial
  Note  Artificial = synthetic construct
WWWWWRWWWWWR

SEQ ID NO: 325 - protein, 18 aa, Artificial
  Note  Artificial = synthetic construct
WWWWWRWWWWWRWWWWWR

SEQ ID NO: 326 - protein, 12 aa, Artificial
  Note  Artificial = synthetic construct
RWWWWWRWWWWW

SEQ ID NO: 327 - protein, 18 aa, Artificial
  Note  Artificial = synthetic construct
RWWWWWRWWWWWRWWWWW

SEQ ID NO: 328 - protein, 12 aa, Artificial
  Note  Artificial = synthetic construct
WRWWWWWWRWWW

SEQ ID NO: 329 - protein, 18 aa, Artificial
  Note  Artificial = synthetic construct
WRWWWWWWRWWWWWWRWW

SEQ ID NO: 330 - protein, 12 aa, Artificial
  Note  Artificial = synthetic construct
WRWWWWWWWWRW

SEQ ID NO: 331 - protein, 18 aa, Artificial
  Note  Artificial = synthetic construct
WRWWWWWWWWRWWWWWWR

SEQ ID NO: 332 - protein, 6 aa, Artificial
  Note  Artificial = synthetic construct
WRWWWW

SEQ ID NO: 333 - protein, 6 aa, Artificial
  Note  Artificial = synthetic construct
WRWWWW

SEQ ID NO: 334 - protein, 6 aa, Artificial
  Note  Artificial = synthetic construct
WRWWWW

SEQ ID NO: 335 - protein, 6 aa, Artificial
  Note  Artificial = synthetic construct
WRWWWW

SEQ ID NO: 336 - protein, 6 aa, Artificial
  Note  Artificial = synthetic construct
WRWWWW

SEQ ID NO: 337 - protein, 6 aa, Artificial
  Note  Artificial = synthetic construct
WRWWWW

SEQ ID NO: 338 - protein, 6 aa, Artificial
  Note  Artificial = synthetic construct
WWRWWW

SEQ ID NO: 339 - protein, 6 aa, Artificial
  Note  Artificial = synthetic construct
WWRWWW

SEQ ID NO: 340 - protein, 6 aa, Artificial
  Note  Artificial = synthetic construct
WWRWWW

SEQ ID NO: 341 - protein, 6 aa, Artificial
  Note  Artificial = synthetic construct
WWRWWW

SEQ ID NO: 342 - protein, 6 aa, Artificial
  Note  Artificial = synthetic construct
WWRWWW

SEQ ID NO: 343 - protein, 6 aa, Artificial
  Note  Artificial = synthetic construct
WWRWWW

SEQ ID NO: 344 - protein, 6 aa, Artificial
  Note  Artificial = synthetic construct
WWWRWW

SEQ ID NO: 345 - protein, 6 aa, Artificial
  Note  Artificial = synthetic construct
WWWRWW

SEQ ID NO: 346 - protein, 6 aa, Artificial
  Note  Artificial = synthetic construct
WWWRWW

SEQ ID NO: 347 - protein, 6 aa, Artificial
  Note  Artificial = synthetic construct
WWWRWW

SEQ ID NO: 348 - protein, 6 aa, Artificial
  Note  Artificial = synthetic construct
WWWRWW

SEQ ID NO: 349 - protein, 6 aa, Artificial
  Note  Artificial = synthetic construct
WWWRWW

SEQ ID NO: 350 - protein, 41 aa, Artificial; identical to SEQ ID NO: 40
  Note  Artificial = synthetic construct

SEQ ID NO: 351 - protein, 41 aa, Artificial; identical to SEQ ID NO: 49
  Note  Artificial = synthetic construct

SEQ ID NO: 352 - protein, 41 aa, Artificial; identical to SEQ ID NO: 58
  Note  Artificial = synthetic construct

SEQ ID NO: 353 - protein, 41 aa, Artificial; identical to SEQ ID NO: 65
  Note  Artificial = synthetic construct

SEQ ID NO: 354 - protein, 41 aa, Artificial; identical to SEQ ID NO: 72
  Note  Artificial = synthetic construct

SEQ ID NO: 355 - protein, 41 aa, Artificial; identical to SEQ ID NO: 80
  Note  Artificial = synthetic construct

SEQ ID NO: 356 - protein, 41 aa, Artificial; identical to SEQ ID NO: 97
  Note  Artificial = synthetic construct

SEQ ID NO: 357 - protein, 41 aa, Artificial; identical to SEQ ID NO: 105
  Note  Artificial = synthetic construct

SEQ ID NO: 358 - protein, 41 aa, Artificial; identical to SEQ ID NO: 106
  Note  Artificial = synthetic construct

SEQ ID NO: 359 - protein, 41 aa, Artificial; identical to SEQ ID NO: 107
  Note  Artificial = synthetic construct

SEQ ID NO: 360 - protein, 41 aa, Artificial; identical to SEQ ID NO: 108
  Note  Artificial = synthetic construct

SEQ ID NO: 361 - protein, 41 aa, Artificial; identical to SEQ ID NO: 109
  Note  Artificial = synthetic construct

SEQ ID NO: 362 - protein, 41 aa, Artificial; identical to SEQ ID NO: 110
  Note  Artificial = synthetic construct

SEQ ID NO: 363 - protein, 48 aa, Artificial; identical to SEQ ID NO: 237
  Note  Artificial = synthetic construct

SEQ ID NO: 364 - protein, 48 aa, Artificial; identical to SEQ ID NO: 240
  Note  Artificial = synthetic construct

SEQ ID NO: 365 - protein, 66 aa, Artificial
  Note  Artificial = synthetic construct
PRGGSVLVTLPSLKGEEMRDRARAHVDALRTHAKPVLLESFKVSFLSALEEYTKKALSPLWRWWWW

SEQ ID NO: 366 - protein, 59 aa, Artificial; identical to SEQ ID NO: 274
  Note  Artificial = synthetic construct

SEQ ID NO: 367 - protein, 66 aa, Artificial; identical to SEQ ID NO: 138
  Note  Artificial = synthetic construct

SEQ ID NO: 368 - protein, 55 aa, Artificial; identical to SEQ ID NO: 275
  Note  Artificial = synthetic construct

SEQ ID NO: 369 - protein, 48 aa, Artificial; identical to SEQ ID NO: 243
  Note  Artificial = synthetic construct

SEQ ID NO: 370 - protein, 59 aa, Artificial; identical to SEQ ID NO: 286
  Note  Artificial = synthetic construct

SEQ ID NO: 371 - protein, 55 aa, Artificial; identical to SEQ ID NO: 287
  Note  Artificial = synthetic construct

SEQ ID NO: 372 - protein, 41 aa, Artificial; identical to SEQ ID NO: 89
  Note  Artificial = synthetic construct

SEQ ID NO: 373 - protein, 6 aa, Artificial
  Note  Artificial = synthetic construct
WKYMVM

(19) United States
(12) Patent Application Publication
(10) Pub. No.: US 2008/0138284 A1
(43) Pub. Date: Jun. 12, 2008

(54) Novel Peptides That Promote Lipid Efflux

(75) Inventor: H. Bryan Brewer, Potomac, MD (US)

Correspondence Address: JOHN S. PRATT, ESQ;KILPATRICK STOCKTON, LLP, 1100 PEACHTREE STREET, ATLANTA, GA, 30309, US

(73) Assignee: Lipid Sciences, Inc., Pleasanton, CA (US)

(21) Appl. No.: 11/861,758

(22) Filed: Sep. 26, 2007

Related U.S. Application Data
(60) Provisional application No. 60/847,578, filed on Sep. 26, 2006.
(60) Provisional application No. 60/847,654, filed on Sep. 26, 2006.
(60) Provisional application No. 60/847,576, filed on Sep. 26, 2006.
(60) Provisional application No. 60/858,045, filed on Nov. 10, 2006.

(51) Int. Cl. A61K 49/00; C07K 14/00; A61K 38/16; A61P 35/00

(52) U.S. Cl. 424/9.1; 530/324; 514/12

(57) ABSTRACT

Disclosed herein are peptides with domains that promote lipid efflux from cells and optionally possess at least one anti-inflammatory domain or a domain that stimulates LCAT activity. Provided herein are methods of using the peptides to treat or inhibit diseases including dyslipidemic disorders, stroke and myocardial infarction. Also provided are methods of detecting plaque in vessels using the labeled peptides of the present invention.

### PRIOR RELATED APPLICATIONS

This application claims the priority benefit of U.S. Provisional Patent Application Nos. 60/847,578, 60/847,654 and 60/847,576 filed Sep. 26, 2006 and 60/858,045 filed Nov. 10, 2006, which are incorporated by reference herein in their entirety.

### FIELD OF THE INVENTION

This present invention relates to peptides and peptide analogs that contain functional domains and promote lipid efflux. These peptides and peptide analogs optionally contain one or more anti-inflammatory domain and one or more domain that affects lecithin cholesterol acyltransferase (LCAT) activity. The disclosure further relates to methods for administering these peptides in the treatment and prevention of dyslipidemic and vascular disorders. The disclosure further relates to methods for using these peptides and peptide analogs in assays and in methods of imaging sites of association of these peptides with receptors and with sites of lipid deposition.

### BACKGROUND OF THE INVENTION

Clearance of excess cholesterol from cells by high density lipoproteins (HDL) is facilitated by the interaction of HDL apolipoprotein with cell-surface binding sites or receptors. Research has demonstrated an inverse correlation between the occurrence of atherosclerosis events and levels of HDL and its most abundant protein constituent, apolipoprotein A-I (apoA-I) (Panagotopulos et al., J. Biol. Chem. 277:39477-39484, 2002). ApoA-I has been shown to promote lipid efflux from ABCA1-transfected cells (Wang et al., J. Biol. Chem. 275:33053-33058, 2000; Hamon et al., Nat. Cell Biol. 2:399-406, 2000; and Remaley et al., Biochem. Biophys. Res. Commun. 280:818-823, 2001). However, the nature of the interaction between apoA-I and ABCA1 is not fully understood.

There exists a need for non-cytotoxic, synthetic peptide mimetics of apolipoproteins that promote specific lipid efflux from cells, perhaps by an ABCA1-dependent pathway, for use in the treatment and prevention of cardiovascular diseases, such as atherosclerosis.

Inflammation is believed to contribute to a variety of disease processes, including vascular disease. Inflammation is believed to contribute to the process of atherosclerosis, and physicians often prescribe anti-inflammatory medicine, such as aspirin, to patients with atherosclerosis, in conjunction with statins, in an attempt to decrease the ongoing inflammatory process that contributes to atherosclerosis and vascular disease. What is needed are compounds that decrease inflammation.

LCAT is the major enzyme involved in the esterification of free cholesterol present in circulating plasma lipoproteins, and a major determinant of plasma HDL concentrations. What is needed are compounds that increase LCAT activity.

What is needed are new compositions that promote lipid efflux. What is also needed are new compositions with functional domains that promote lipid efflux and have anti-inflammatory properties and/or activity to modulate LCAT activity, or a combination of domains that have anti-inflammatory properties and the activity to modulate LCAT activity.

### SUMMARY OF THE INVENTION

The present invention solves these problems by providing novel peptide compositions with functional domains. In several embodiments, these novel peptide compositions promote lipid efflux and/or have anti-inflammatory properties. In several embodiments, these novel peptide compositions promote lipid efflux and have one or more anti-inflammatory domains. In several embodiments, these novel peptide compositions promote lipid efflux and have one or more domains that affect LCAT activity. In several embodiments, these novel peptide compositions promote lipid efflux and have one or more anti-inflammatory domains and one or more domains that affect LCAT activity.

These novel peptide compositions may be labeled and used in a variety of applications including the visualization of plaque in vessels. These novel peptide compositions also display low toxicity.

The peptides of the present invention may be combined with pharmaceutically acceptable carriers and administered to a human or an animal as a composition. The peptides of the present invention may also be reconstituted with lipids and or sphingomyelin before administration. Administration may be through any means described herein and includes but is not limited to parenteral and oral administration and also administration on a coated device such as a stent or catheter. It is to be understood that any of the peptides of the present invention may be synthesized to include D-amino acids, instead of L-amino acids. Such peptides containing D-amino acids are particularly suitable for oral administration as the presence of D-amino acids reduces degradation. Some of the peptides of the present invention may contain a D-amino acid at the carboxyl terminal, at the amino-terminal, or at both the carboxyl terminal and the amino-terminal.

Also described herein is a method of treating dyslipidemic and vascular disorders in an animal or a human, including administering to the animal or the human a therapeutically effective amount of the peptides or peptide analogs thereof presented herein. Dyslipidemic and vascular disorders amenable to treatment with the peptides disclosed herein include, but are not limited to, hyperlipidemia, hyperlipoproteinemia, hypercholesterolemia, hypertriglyceridemia, HDL deficiency, apoA-I deficiency, coronary artery disease, atherosclerosis, myocardial infarction, stroke and inflammation secondary to stroke, ischemia, ischemic stroke, thrombotic stroke, peripheral vascular disease including peripheral arterial disease, restenosis, thrombosis, acute coronary syndrome, and reperfusion myocardial injury.

The peptides of the present invention may be labeled with labels known to one of ordinary skill in the art and used for numerous applications, including but not limited to use in imaging applications to visualize atherosclerotic plaque. Labels include but are not limited to colorimetric labels, radiodense labels and radioisotopic labels. Other uses include but are not limited to use in assays, such as ELISAs, Western blots, radioimmunoassays and radioreceptor assays.

The peptides of the present invention may be used to generate antisera using techniques known to one of ordinary skill in the art.

The amino acid sequences disclosed herein are shown using standard three letter codes for amino acids, as defined in 37 C.F.R. 1.822 and as commonly known to one of ordinary skill in the art. When the three letter designation for an amino acid is shown in three upper case letters, for example SER for serine, the SER is a D amino acid.

Several of the generic formulae described below refer to helical regions 2, 5, 6, 7, and 8 of ApoA-I. Helices 2, 5, 6, 7 and 8 of ApoA-I are defined as follows, wherein each helix number is followed by the amino acid residues of ApoA-I associated with that helix: helix 2: amino acid residues 68-85; helix 5: amino acid residues 145-162; helix 6: amino acid residues 167-184; helix 7: amino acid residues 189-206; and, helix 8: amino acid residues 222-239. FIG. 1 shows the numbered amino acid sequence of ApoA-I.

In one embodiment, the peptides of the present invention are described by the following generic formula I:

(A-B-C)ₙ I.

wherein A comprises helix 2 of ApoA-I, helix 5 of ApoA-I, helix 6 of ApoA-I, helix 7 of ApoA-I, or a modified form of helix 8 of ApoA-I (hereinafter called 8′), C comprises helix 8 of ApoA-I, B is a linking group between A and C, and n is an integer from 1 to 10.

In one embodiment, A is helix 2 of ApoA-I and is SEQ ID NO:1 Thr Gln Glu Phe Trp Asp Asn Leu Glu Lys Glu Thr Glu Gly Leu Arg Gln Glu, or a variation or conservative substitution thereof. These amino acids may also appear in reverse orientation as in SEQ ID NO:2 Glu Gln Arg Leu Gly Glu Thr Glu Lys Glu Leu Asn Asp Trp Phe Glu Gln Thr.

In another embodiment, A is helix 5 of ApoA-I and is SEQ ID NO:3 Gly Glu Glu Met Arg Asp Arg Ala Arg Ala His Val Asp Ala Leu Arg Thr His, or a variation or conservative substitution thereof. These amino acids may also appear in reverse orientation as in SEQ ID NO:4 His Thr Arg Leu Ala Asp Val His Ala Arg Ala Arg Asp Arg Met Glu Glu Gly.

In another embodiment, A is helix 6 of ApoA-I and is SEQ ID NO:5 Ser Asp Glu Leu Arg Gln Arg Leu Ala Ala Arg Leu Glu Ala Leu Lys Glu Asn, or a variation or conservative substitution thereof. These amino acids may also appear in reverse orientation as in SEQ ID NO:6 Asn Glu Lys Leu Ala Glu Leu Arg Ala Ala Leu Arg Gln Arg Leu Glu Asp Ser.

In another embodiment, A is helix 7 of ApoA-I and is SEQ ID NO:7 Leu Ala Glu Tyr His Ala Lys Ala Thr Glu His Leu Ser Thr Leu Ser Glu Lys, or a variation or a conservative substitution thereof. These amino acids may also appear in reverse orientation as in SEQ ID NO:8 Lys Glu Ser Leu Thr Ser Leu His Glu Thr Ala Lys Ala His Tyr Glu Ala Leu.

In another embodiment, A is a modified form of helix 8 of ApoA-I, (hereinafter called 8′), and is SEQ ID NO:9 Leu Glu Ser Ala Lys Val Ser Ala Leu Ser Ala Leu Glu Glu Ala Thr Lys Lys, or a variation or a conservative substitution thereof. These amino acids may also appear in reverse orientation such that Lys is at the N-terminus and Leu is at the C-terminus as in SEQ ID NO:10 Lys Lys Thr Ala Glu Glu Leu Ala Ser Leu Ala Ser Val Lys Ala Ser Glu Leu. This modification of helix 8 involves substitutions at positions 4 (Phe to Ala), 8 (Phe to Ala) and 15 (Tyr to Ala). It is to be understood that the present invention encompasses other amino acid substitutions at these locations. At positions 4 and 8, Phe may be substituted with Val, Leu, Gly, Thr, Ser or gamma aminobutyric acid (GABA: GABA is also designated as 4Abu herein). At position 15, Tyr may be substituted with Val, Leu, Gly, Thr, Ser or GABA. While not wanting to be bound by the following statement, it is believed that A, the modified form of helix 8 of ApoA-I, has a lower lipid affinity than C, the unmodified form of helix 8 of ApoA-I.

In one embodiment, when A is helix 2, helix 5, helix 6, helix 7 or helix 8′, and C is helix 8, B is SEQ ID NO:11 Ala Lys Pro Val Leu, SEQ ID NO:12 Leu Val Pro Lys Ala, SEQ ID NO:13 Lys Pro Val Leu, SEQ ID NO:14 Pro Val Leu, Pro Val, Pro, SEQ ID NO:15 Ala Lys Pro Val, SEQ ID NO:16 Ala Lys Pro, SEQ ID NO:17 Lys Pro Val or Lys Pro.

In one embodiment, when A helix 2 or helix 7, and C is helix 8, B is Pro, SEQ ID NO:18 Lys Leu Ser Pro Leu, SEQ ID NO:19 Leu Ser Pro Leu, or SEQ ID NO:20 Ser Pro Leu, or a conservative substitution thereof. These amino acids in B may also appear in reverse orientation for example as in SEQ ID NO:21 Leu Pro Ser Leu Lys, SEQ ID NO:22 Leu Pro Ser Leu, and SEQ ID NO:23 Leu Pro Ser.

However, when A is helix 5, helix 6, or helix 8′ and C is helix 8, B is not Pro, SEQ ID NO:18 Lys Leu Ser Pro Leu, SEQ ID NO:19 Leu Ser Pro Leu, SEQ ID NO:20 Ser Pro Leu, SEQ ID NO:21 Leu Pro Ser Leu Lys, SEQ ID NO:22 Leu Pro Ser Leu, or SEQ ID NO:23 Leu Pro Ser or a conservative substitution thereof.

In one embodiment, C is helix 8 of ApoA-I and is SEQ ID NO:24 Leu Glu Ser Phe Lys Val Ser Phe Leu Ser Ala Leu Glu Glu Tyr Thr Lys Lys, or a variation or conservative substitution thereof. These amino acids may also appear in reverse orientation such that Lys is at the N-terminus and Leu is at the C-terminus as SEQ ID NO:25 Lys Lys Thr Tyr Glu Glu Leu Ala Ser Leu Phe Ser Val Lys Phe Ser Glu Leu.

It is to be understood that A and C may be switched in location as in C-B-A.

In a further embodiment, peptides of the present invention are described by the following subgeneric formula II, in which one or more additional elements indicated as variables D, E, F and W, are added to formula I to make subgeneric formula II.

D-E-(A-B-C)ₙ-F-W II.

(A-B-C)ₙ are as described in formula I above.

D is absent or present and is a peptide as defined in the present specification. In one embodiment, D is a peptide selected from the group consisting of SEQ ID NO:26 Pro Arg Gly Gly Ser Val Leu Val Thr, or multiples, variations or conservative substitutions thereof. These amino acids may also appear in reverse orientation, namely SEQ ID NO:27 Thr Val Leu Val Ser Gly Gly Arg Pro. It is to be understood that one or more of the first six N-terminal amino acids of D, namely SEQ ID NO:28 Pro Arg Gly Gly Ser Val or SEQ ID NO:29 Thr Val Leu Val Ser Gly may occur as D-amino acids;

E is absent or present and is a group linking D and A and is Pro, SEQ ID NO:21 Leu Pro Ser Leu Lys, SEQ ID NO:30 Pro Lys Ala or a conservative substitution thereof, provided that E is present only when D is present. These amino acids may also appear in reverse orientation as in SEQ ID NO:11 Ala Lys Pro Val Leu and SEQ ID NO:16 Ala Lys Pro. When D is absent, E is absent.

F is absent or present and is a group linking C and W and is Pro, SEQ ID NO:31 Ala Leu Ser Pro Leu, SEQ ID NO:11 Ala Lys Pro Val Leu or a conservative substitution thereof, provided that F is present only when W is present. These amino acids may also appear in reverse orientation as in SEQ ID NO:32 Leu Pro Ser Leu Ala and SEQ ID NO:12 Leu Val Pro Lys Ala. When W is absent, F is absent.

W is absent or present and is a peptide as defined in the present specification. In one embodiment, W is a peptide selected from the group consisting of SEQ ID NO:33 Trp Arg Trp Trp Trp Trp, or multiples, variations or conservative substitutions thereof. These amino acids may also appear in reverse orientation, namely SEQ ID NO:34 Trp Trp Trp Trp Arg Trp. It is to be understood that one or more of the amino acids in the W peptide may be D amino acids.

As stated above, in generic formula II, variables D or W may be absent or present. In one embodiment, D is present and W is absent. In another embodiment, W is present and D is absent. In another embodiment, both D and W are present.

In yet another embodiment, W and D as described in formula II may be switched in location to produce subgeneric formula III.

W-E-(A-B-C)ₙ-F-D III.

In a further embodiment, peptides of the present invention are described by the following subgeneric formula IV, in which one or more additional elements indicated as variables G and H, are added to formula I to make subgeneric formula IV.

G-(A-B-C)ₙ-H IV.

(A-B-C)n are as described in formula I above,

G is absent or present and is a peptide as defined in the present specification. In one embodiment, G is SEQ ID NO:20 Ser Pro Leu or a conservative substitution thereof. These amino acids may also appear in reverse orientation as in SEQ ID NO:23 Leu Pro Ser. It is to be understood that one or more of the amino acids in the G peptide may be D amino acids.

H is absent or present and is a peptide as defined in the present specification. In one embodiment, H is SEQ ID NO:35 Leu Asn Thr Gln or a conservative substitution thereof. These amino acids may also appear in reverse orientation as in SEQ ID NO:36 Gln Thr Asn Leu. It is to be understood that one or more of the amino acids in the H peptide may be D amino acids.

In a further embodiment, peptides of the present invention are described by the following subgeneric formula V, in which one or more additional elements indicated as variables D, E, F, W, G and H are added to formula I to make subgeneric formula V.

D-E-G-(A-B-C)ₙ-H-F-W V.

(A-B-C)ₙ are as described in formula I above,

D is absent or present and is a peptide as defined in the present specification. In one embodiment, D is a peptide selected from the group consisting of SEQ ID NO:26 Pro Arg Gly Gly Ser Val Leu Val Thr, or multiples, variations or conservative substitutions thereof. These amino acids may also appear in reverse orientation, namely SEQ ID NO:27 Thr Val Leu Val Ser Gly Gly Arg Pro. It is to be understood that one or more of the first six N-terminal amino acids of D, namely SEQ ID NO:28 Pro Arg Gly Gly Ser Val or SEQ ID NO:29 Thr Val Leu Val Ser Gly may occur as D-amino acids;

E is absent or present and is a group linking D and A and is Pro, SEQ ID NO:21 Leu Pro Ser Leu Lys, SEQ ID NO:30 Pro Lys Ala or a conservative substitution thereof, provided that E is present only when D is present. These amino acids may also appear in reverse orientation as in SEQ ID NO:11 Ala Lys Pro Val Leu and SEQ ID NO:16 Ala Lys Pro. When D is absent, E is absent.

F is absent or present and is a group linking C and W and is Pro, SEQ ID NO:31 Ala Leu Ser Pro Leu, SEQ ID NO:11 Ala Lys Pro Val Leu or a conservative substitution thereof, provided that F is present only when W is present. These amino acids may also appear in reverse orientation as in SEQ ID NO:32 Leu Pro Ser Leu Ala and SEQ ID NO:12 Leu Val Pro Lys Ala. When W is absent, F is absent.

W is absent or present and is a peptide as defined in the present specification. In one embodiment, W is a peptide selected from the group consisting of SEQ ID NO:33 Trp Arg Trp Trp Trp Trp, or multiples, variations or conservative substitutions thereof. These amino acids may also appear in reverse orientation, namely SEQ ID NO:34 Trp Trp Trp Trp Arg Trp. It is to be understood that one or more of the amino acids in the W peptide may be D amino acids.

G is absent or present and is a peptide as defined in the present specification provided that G is present when D is absent. In one embodiment, G is SEQ ID NO:20 Ser Pro Leu or a conservative substitution thereof. These amino acids may also appear in reverse orientation as in SEQ ID NO:23 Leu Pro Ser. It is to be understood that one or more of the amino acids in the G peptide may be D amino acids.

H is absent or present and is a peptide as defined in the present specification provided that H is present when W is absent. In one embodiment, H is SEQ ID NO:35 Leu Asn Thr Gln or a conservative substitution thereof. These amino acids may also appear in reverse orientation as in SEQ ID NO:36 Gln Thr Asn Leu. It is to be understood that one or more of the amino acids in the H peptide may be D amino acids.

As stated above, in generic formula V, variables D or W may be absent or present. In one embodiment, D is present and W is absent. In another embodiment, W is present and D is absent. In another embodiment, both D and W are present.

In a further embodiment, peptides of the present invention are described by formula VI,

D-I-W VI.

D is a peptide as defined in the present specification. In one embodiment, D is a peptide selected from the group consisting of SEQ ID NO:26 Pro Arg Gly Gly Ser Val Leu Val Thr, or multiples, variations or conservative substitutions thereof. These amino acids may also appear in reverse orientation, namely SEQ ID NO:27 Thr Val Leu Val Ser Gly Gly Arg Pro. It is to be understood that one or more of the first six N-terminal amino acids of D, namely SEQ ID NO:28 Pro Arg Gly Gly Ser Val or SEQ ID NO:29 Thr Val Leu Val Ser Gly may occur as D-amino acids;

I is a group linking D and W and is SEQ ID NO:37 Leu Ala Lys Pro Val Leu or a conservative substitution thereof, or multiples thereof or combinations thereof. These amino acids may also appear in reverse orientation, for example, SEQ ID NO:38 Leu Val Pro Lys Ala Leu.

W is absent or present and is a peptide as defined in the present specification. In one embodiment, W is a peptide selected from the group consisting of SEQ ID NO:33 Trp Arg Trp Trp Trp Trp, or multiples, variations or conservative substitutions thereof. These amino acids may also appear in reverse orientation, namely SEQ ID NO:34 Trp Trp Trp Trp Arg Trp. It is to be understood that one or more of the amino acids in the W peptide may be D amino acids.

D and W may also be switched in location in D-I-W to form W-I-D.

It is to be understood that the letters in the generic formulae I through VI or in components thereof are defined by the text that follows each letter and do not designate an individual amino acid.

It is to be understood that in some embodiments, one or more of the amino acids of the peptides of the present invention are D amino acids. In one embodiment, the N-terminal amino acid, the C-terminal amino acid, or both, are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention.

The N and/or C-terminal amino acids may also be modified by amidation, acetylation or other modifications known to one of ordinary skill in the art. The peptides of the present invention may optionally be acetylated at the N-terminus or the C-terminus using techniques known to one of ordinary skill in the art. The peptides of the present invention may optionally be amidated at the N-terminus or the C-terminus using techniques known to one of ordinary skill in the art. In one embodiment, the peptides of the present invention are acetylated at the N-terminus, amidated at the C-terminus, or both acetylated at the N-terminus and amidated at the C-terminus. In some embodiments, the peptides of the present invention may have both an acetylated N-terminus and a carboxy terminal amide. In the present application, when a peptide is acetylated on an N or C terminus, the letters Ac are indicated. In the present application, when a peptide is amidated on an N or C terminus, the designation NH₂ is employed.

The present invention also includes compositions comprising one or more individual peptides of the present invention in an acceptable carrier. These peptides are as defined above and may be labeled or unlabelled. It is to be understood that a mixture of peptides, may include different amounts of the individual peptides. For example, in one embodiment, each peptide component of the combination may be present in a different relative percentage than each other peptide component due to differences in relative efficacy to promote lipid efflux or to provide one or more types of anti-inflammatory activity.

Accordingly, it is an object of the present invention to provide novel peptides.

Accordingly, it is an object of the present invention to provide novel peptides that facilitate lipid efflux.

Yet another object of the present invention is to provide novel peptides that facilitate lipid efflux and possess anti-inflammatory biological activity.

Still another object of the present invention is to provide novel peptides that facilitate lipid efflux and stimulate LCAT activity.

Yet another object of the present invention is to provide novel peptides that facilitate lipid efflux, possess anti-inflammatory biological activity, and stimulate LCAT activity.

It is another object of the present invention to provide new methods for visualizing plaque using labeled peptides of the present invention.

It is yet another object of the present invention to provide new methods for the treatment of atherosclerosis, cardiovascular disease and cerebrovascular disease in an animal or a human by administering pharmaceutical compositions comprising one or more peptides of the present invention with a pharmaceutically acceptable carrier, or on a medical device.

These and other objects, features and advantages of the present invention will become apparent after a review of the following detailed description of the disclosed embodiments and claims.

### BRIEF DESCRIPTION OF THE FIGURES

FIG. 1 shows the amino acid sequence of ApoA-I (SEQ ID NO:39).

### DETAILED DESCRIPTION OF THE PRESENT INVENTION

The present invention provides novel peptides. The present invention solves the problems described above by providing novel peptide compositions with functional domains. In some embodiments, these novel peptide compositions promote lipid efflux. In some embodiments, these novel peptide compositions promote lipid efflux and have anti-inflammatory properties. In other embodiments, these novel peptide compositions promote lipid efflux and have one or more anti-inflammatory domains. In yet other embodiments, these novel peptide compositions promote lipid efflux and have one or more domains that affect LCAT activity. In several embodiments, these novel peptide compositions promote lipid efflux and have one or more anti-inflammatory domains and one or more domain that affects LCAT activity.

Any of the peptides of the present invention may optionally be acetylated at the N-terminus or the C-terminus using techniques known to one of ordinary skill in the art. The peptides of the present invention may optionally be amidated at the N-terminus or the C-terminus using techniques known to one of ordinary skill in the art. In one embodiment, the peptides of the present invention are acetylated at the N-terminus, amidated at the C-terminus, or both acetylated at the N-terminus and amidated at the C-terminus. In some embodiments, the peptides of the present invention may have both an acetylated N-terminus and a carboxy terminal amide. In the present application, when a peptide is acetylated on an N or C terminus, the letters Ac are indicated. In the present application, when a peptide is amidated on an N or C terminus, the designation NH₂ is employed.

One or more of these peptides may be combined with an acceptable carrier and administered as compositions to individuals in order to provide lipid efflux and anti-inflammatory activities. These compositions may be administered to treat dyslipidemic and vascular disorders or to delay or prevent the onset or progression of dyslipidemic and vascular disorders. In one embodiment, these compositions may be administered to treat atherosclerosis or to delay or prevent its onset or progression. These novel peptide compositions may be labeled and used in a variety of applications including the visualization of plaque in vessels. These novel peptide compositions also display low toxicity.

### I. Abbreviations

ABCA1: ATP-binding cassette transporter Al

apoA-I: apolipoprotein A-I

DMPC: dimyristoyl phosphatidyl choline

HDL: high-density lipoprotein

HPLC: high-pressure liquid chromatography

LDL: low-density lipoprotein

RBC: red blood cell

### II. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

In order to facilitate review of the various embodiments of this disclosure, the following explanations of specific terms are provided:

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Animal: Living multi-cellular vertebrate organisms, a category that includes, for example, mammals and birds. The term mammal includes both human and non-human mammals. Similarly, the term “subject” includes both human and veterinary subjects, for example, humans, non-human primates, dogs, cats, horses, and cows.

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kDa) and one “heavy” (about 50-70 kDa) chain. The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

As used herein, the term “antibody” includes intact immunoglobulins as well as a number of well-characterized fragments. For instance, Fabs, Fvs, and single-chain Fvs (SCFvs) that bind to target protein (or epitope within a protein or fusion protein) would also be specific binding agents for that protein (or epitope). These antibody fragments are as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody, a genetically engineered molecule containing the variable region of the light chain, the variable region of the heavy chain, linked by a suitable polypeptide linker as a genetically fused single chain molecule. Methods of making these fragments are routine (see, e.g., Harlow and Lane, Using Antibodies: A Laboratory Manual, CSHL, New York, 1999).

Antibodies for use in the methods and compositions of this disclosure can be monoclonal or polyclonal. Merely by way of example, monoclonal antibodies can be prepared from murine hybridomas according to the classical method of Kohler and Milstein (Nature 256:495-97, 1975) or derivative methods thereof. Detailed procedures for monoclonal antibody production are described in Harlow and Lane, Using Antibodies: A Laboratory Manual, CSHL, New York, 1999.

Domain: A domain of a protein is a part of a protein that shares common structural, physiochemical and functional features; for example hydrophobic, polar, globular, helical domains or properties, for example a DNA binding domain, an ATP binding domain, an anti-inflammatory domain, an LCAT activating domain and the like. Some peptides of the present invention possess a domain or domains that have more than one functional feature, for example both lipid efflux activity and anti-inflammatory activity.

Dyslipidemic disorder: A disorder associated with any altered amount of any or all of the lipids or lipoproteins in the blood. Dyslipidemic disorders include, for example, hyperlipidemia, hyperlipoproteinemia, hypercholesterolemia, hypertriglyceridemia, HDL deficiency, apoA-I deficiency, and cardiovascular disease (e.g., coronary artery disease, atherosclerosis and restenosis).

Efflux: The process of flowing out. As applied to the results described herein, lipid efflux refers to a process whereby lipid, such as cholesterol and phospholipid, is complexed with an acceptor, such as an apolipoprotein or apolipoprotein peptide mimetic, or a peptide of the present invention and removed from vesicles or cells. “ABCA1-dependent lipid efflux” (or lipid efflux by an “ABCAl-dependent pathway”) refers to a process whereby apolipoproteins, synthetic peptide mimetics of apolipoproteins, or a peptide of the present invention, bind to a cell and efflux lipid from the cell by a process that is facilitated by the ABCAl transporter.

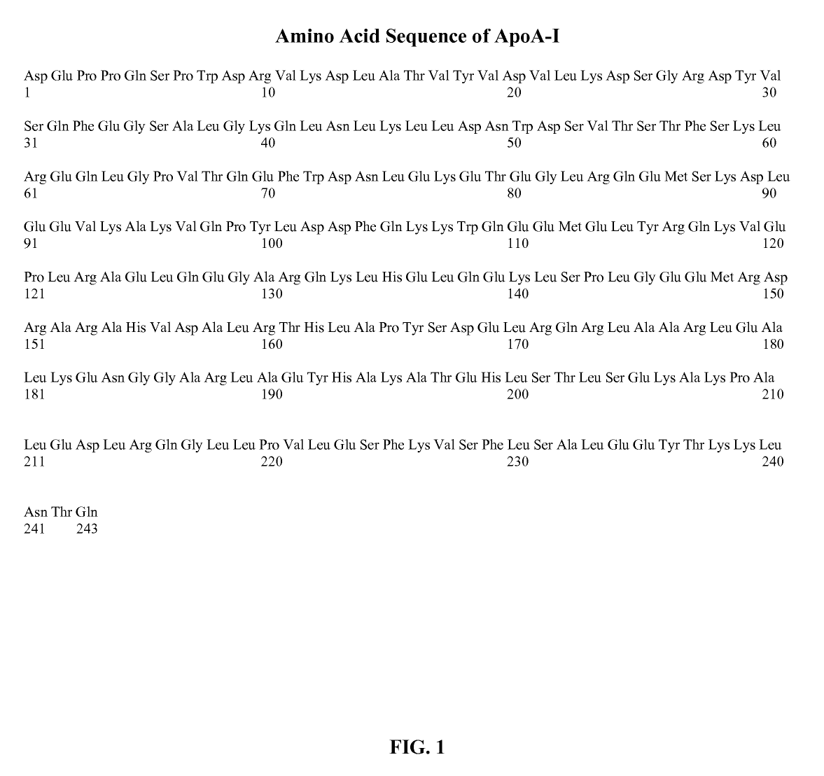

Helix: The molecular conformation of a spiral nature, generated by regularly repeating rotations around the backbone bonds of a macromolecule. Helices 2, 5, 6, 7 and 8 of ApoA-I are defined as follows, wherein each helix number is followed by the amino acid residues of ApoA-I associated with that helix: helix 2: amino acid residues 68-85; helix 5: amino acid residues 145-162; helix 6: amino acid residues 167-184; helix 7: amino acid residues 189-206; and, helix 8: amino acid residues 222-239. FIG. 1 shows the numbered amino acid sequence of ApoA-I (SEQ ID NO:39).

Hydrophobic: A hydrophobic (or lipophilic) group is electrically neutral and nonpolar, and thus prefers other neutral and nonpolar solvents or molecular environments. Examples of hydrophobic molecules include alkanes, oils and fats.

Hydrophilic: A hydrophilic (or lipophobic) group is electrically polarized and capable of H-bonding, enabling it to dissolve more readily in water than in oil or other “non-polar” solvents.

Inhibiting or treating a disease: Inhibiting the full development of a disease, disorder or condition, for example, in a subject who is at risk for a disease such as atherosclerosis and cardiovascular disease. “Treatment” refers to a therapeutic intervention that ameliorates a sign or symptom of a disease or pathological condition after it has begun to develop. As used herein, the term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease.

Isolated/purified: An “isolated” or “purified” biological component (such as a nucleic acid, peptide or protein) has been substantially separated, produced apart from, or purified away from other biological components in the cell of the organism in which the component naturally occurs, that is, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a host cell as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell. Preferably, a preparation is purified such that the biological component represents at least 50%, such as at least 70%, at least 90%, at least 95%, or greater of the total biological component content of the preparation.

Label: A detectable compound or composition that is conjugated directly or indirectly to another molecule to facilitate detection of that molecule. Specific, non-limiting examples of labels include fluorescent tags, calorimetric labels, dyes, beads, enzymatic linkages, radiodense materials, and radioactive isotopes.

Linker: A molecule that joins two other molecules, either covalently, or through ionic, van der Waals or hydrogen bonds.

Lipid: A class of water-insoluble, or partially water insoluble, oily or greasy organic substances, that are extractable from cells and tissues by nonpolar solvents, such as chloroform or ether. Types of lipids include triglycerides (e.g., natural fats and oils composed of glycerin and fatty acid chains), glycolipids, phospholipids (e.g., phosphatidylethanolamine, phosphatidylcholine, phosphatidylserine, and phosphatidylinositol), sphingolipids (e.g., sphingomyelin, cerebrosides and gangliosides), and sterols (e.g., cholesterol).

Lipid affinity: A measurement of the relative binding affinity of an amphipathic α-helix for lipids. In some embodiments, the lipid affinity of an amphipathic α-helix is determined by one or more functional tests. Specific, non-limiting examples of functional tests include: retention time on reverse phase HPLC, surface monolayer exclusion pressure (Palgunachari et al., Arterioscler. Thromb. Vasc. Biol. 16:328-338, 1996), binding affinity to phospholipid vesicles (Palgunachari et al., Arterioscler. Thromb. Vasc. Biol. 16:328-338, 1996), and DMPC vesicle solubilization (Remaley et al., J. Lipid Res. 44:828-836, 2003).

Further non-limiting examples of alternative methods of calculating the lipid affinity of an amphipathic α-helix include: total hydrophobic moment, total peptide hydrophobicity, total peptide hydrophobicity per residue, hydrophobicity of amino acids on the hydrophobic face, hydrophobicity per residue of amino acids on the hydrophobic face, and calculated lipid affinity based on predicted peptide penetration into phospholipid bilayers (Palgunachari et al., Arterioscler. Thromb. Vasc. Biol. 16:328-338, 1996).

Non-cytotoxic: A non-cytotoxic compound is one that does not substantially affect the viability or growth characteristics of a cell at a dosage normally used to treat the cell or a subject. Furthermore, the percentage of cells releasing intracellular contents, such as LDH or hemoglobin, is low (e.g., about 10% or less) in cells treated with a non-cytotoxic compound. Lipid efflux from a cell that occurs by a non-cytotoxic compound results in the removal of lipid from a cell by a process that maintains the overall integrity of the cell membrane and does not lead to significant cell toxicity.

Non-polar: A non-polar compound is one that does not have concentrations of positive or negative electric charge. Non-polar compounds, such as, for example, oil, are not well soluble in water.

Peptide: A polymer in which the monomers are amino acid residues which are joined together through amide bonds. When the amino acids are alpha-amino acids, either the L-optical isomer or the D-optical isomer can be used. The amino acid sequences disclosed herein are shown using three letter codes for amino acids, as defined in 37 C.F.R. 1.822 and as commonly known to one of ordinary skill in the art. When the three letter designation for an amino acid, for example Ser for serine is shown in upper case, SER, the serine is a D amino acid. It is to be understood that in some embodiments, one or more of the amino acids of the peptides of the present invention are D amino acids. In one embodiment, the N-terminal amino acid, the C-terminal amino acid, or both, are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention. The terms “peptide” or “polypeptide” as used herein are intended to encompass any amino acid sequence and include modified sequences such as glycoproteins. The term “peptide” is specifically intended to cover naturally occurring peptides, as well as those which are recombinantly or synthetically produced. The term “residue” or “amino acid residue” includes reference to an amino acid that is incorporated into a peptide, polypeptide, or protein. As known to one of skill in the art, the peptides presented herein are read from the N to the C terminus i.e., from left to right. Accordingly, the N terminal amino acid in Leu Glu Lys is Leu and the C-terminal amino acid is Lys. Peptides of the present invention include conservatively substituted peptides, wherein these conservative substitutions occur at 1%, 3%, 5%, 7%, 10%, 15%, 20%, 25%, 30%, 40%, or 50% of the amino acid residues. Peptides of the present invention include peptides that are homologous at 50%, 60%, 70%, 80%, 90%, 95%, 97%, 98%, 99% of the entire sequence of the peptide.

Pharmaceutically acceptable carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as one or more peptides or peptide analogs and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phospholipid: A phospholipid consists of a water-soluble polar head, linked to two water-insoluble non-polar tails (by a negatively charged phosphate group). Both tails consist of a fatty acid, each about 14 to about 24 carbon groups long. When placed in an aqueous environment, phospholipids form a bilayer or micelle, where the hydrophobic tails line up against each other. This forms a membrane with hydrophilic heads on both sides. A phospholipid is a lipid that is a primary component of animal cell membranes.

Polar: A polar molecule is one in which the centers of positive and negative charge distribution do not converge. Polar molecules are characterized by a dipole moment, which measures their polarity, and are soluble in other polar compounds and virtually insoluble in nonpolar compounds.

Recombinant nucleic acid: A sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques such as those described in Sambrook et al. (ed.), Molecular Cloning: A Laboratory Manual, 2^{nd} ed., vol. 1-3, Cold Spring Harbor Laboratory Press, Cold Spring Harbor, N.Y., 1989. The term recombinant includes nucleic acids that have been altered solely by addition, substitution, or deletion of a portion of the nucleic acid.

Therapeutically effective amount: A quantity of a specified agent sufficient to achieve a desired effect in a subject being treated with that agent. For example, this can be the amount of a peptide or peptide analog useful in preventing, ameliorating, and/or treating a dyslipidemic disorder (e.g., atherosclerosis) in a subject. Ideally, a therapeutically effective amount of an agent is an amount sufficient to prevent, ameliorate, and/or treat a dyslipidemic disorder (e.g., atherosclerosis) in a subject without causing a substantial cytotoxic effect (e.g., membrane microsolubilization) in the subject. The effective amount of an agent useful for preventing, ameliorating, and/or treating a dyslipidemic disorder (e.g., atherosclerosis) in a subject will be dependent on the subject being treated, the severity of the disorder, and the manner of administration of the therapeutic composition.

Transformed: A “transformed” cell is a cell into which has been introduced a nucleic acid molecule by molecular biology techniques. The term encompasses all techniques by which a nucleic acid molecule might be introduced into such a cell, including transfection with viral vectors, transformation with plasmid vectors, and introduction of naked DNA by electroporation, lipofection, and particle gun acceleration.

### III. Peptides of the Present Invention and Analogs Thereof

Several of the generic formulae described below refer to helical regions 2, 5, 6, 7, and 8 of ApoA-I. Helices 2, 5, 6, 7 and 8 of ApoA-I are defined as follows, wherein each helix number is followed by the amino acid residues of ApoA-I associated with that helix: helix 2: amino acid residues 68-85; helix 5: amino acid residues 145-162; helix 6: amino acid residues 167-184; helix 7: amino acid residues 189-206; and, helix 8: amino acid residues 222-239. FIG. 1 shows the numbered amino acid sequence of ApoA-I (SEQ ID NO:39).

In one embodiment, the peptides of the present invention are described by the following generic formula I:

(A-B-C)ₙ I.

wherein A comprises helix 2 of ApoA-I, helix 5 of ApoA-I, helix 6 of ApoA-I, helix 7 of ApoA-I, or a modified form of helix 8 of ApoA-I (hereinafer called 8′), C comprises helix 8 of ApoA-I, B is a linking group between A and C, and n is an integer from 1 to 10.

In one embodiment, A is helix 2 of ApoA-I and is SEQ ID NO:1 Thr Gln Glu Phe Trp Asp Asn Leu Glu Lys Glu Thr Glu Gly Leu Arg Gln Glu, or a variation or conservative substitution thereof. These amino acids may also appear in reverse orientation as in SEQ ID NO:2 Glu Gln Arg Leu Gly Glu Thr Glu Lys Glu Leu Asn Asp Trp Phe Glu Gln Thr.

In another embodiment, A is helix 5 of ApoA-I and is SEQ ID NO:3 Gly Glu Glu Met Arg Asp Arg Ala Arg Ala His Val Asp Ala Leu Arg Thr His, or a variation or conservative substitution thereof. These amino acids may also appear in reverse orientation as in SEQ ID NO:4 His Thr Arg Leu Ala Asp Val His Ala Arg Ala Arg Asp Arg Met Glu Glu Gly.

In another embodiment, A is helix 6 of ApoA-I and is SEQ ID NO:5 Ser Asp Glu Leu Arg Gln Arg Leu Ala Ala Arg Leu Glu Ala Leu Lys Glu Asn, or a variation or conservative substitution thereof. These amino acids may also appear in reverse orientation as in SEQ ID NO:6 Asn Glu Lys Leu Ala Glu Leu Arg Ala Ala Leu Arg Gln Arg Leu Glu Asp Ser.

In another embodiment, A is helix 7 of ApoA-I and is SEQ ID NO:7 Leu Ala Glu Tyr His Ala Lys Ala Thr Glu His Leu Ser Thr Leu Ser Glu Lys, or a variation or a conservative substitution thereof. These amino acids may also appear in reverse orientation as in SEQ ID NO:8 Lys Glu Ser Leu Thr Ser Leu His Glu Thr Ala Lys Ala His Tyr Glu Ala Leu.

In another embodiment, A is a modified form of helix 8 of ApoA-I, (hereinafter called 8′), and is SEQ ID NO:9 Leu Glu Ser Ala Lys Val Ser Ala Leu Ser Ala Leu Glu Glu Ala Thr Lys Lys, or a conservative substitution thereof. These amino acids may also appear in reverse orientation such that Lys is at the N-terminus and Leu is at the C-terminus as in SEQ ID NO:10 Lys Lys Thr Ala Glu Glu Leu Ala Ser Leu Ala Ser Val Lys Ala Ser Glu Leu. This modification of helix 8 to create 8′ involves substitutions at positions 4 (Phe to Ala), 8 (Phe to Ala) and 15 (Tyr to Ala). It is to be understood that the present invention encompasses other amino acid substitutions at these locations. At positions 4 and 8, Phe may be substituted with Val, Leu, Gly, Thr, Ser or gamma aminobutyric acid (GABA: GABA is also designated as 4Abu herein). At position 15, Tyr may be substituted with Val, Leu, Gly, Thr, Ser or GABA. While not wanting to be bound by the following statement, it is believed that A, the modified form of helix 8 of ApoA-I, has a lower lipid affinity than C, the unmodified form of helix 8 of ApoA-I.

In one embodiment, when A is helix 2, helix 5, helix 6, helix 7 or helix 8′, and C is helix 8, B is SEQ ID NO:11 Ala Lys Pro Val Leu, SEQ ID NO:12 Leu Val Pro Lys Ala, SEQ ID NO:13 Lys Pro Val Leu, SEQ ID NO:14 Pro Val Leu, Pro Val, Pro, SEQ ID NO:15 Ala Lys Pro Val, SEQ ID NO:16 Ala Lys Pro, SEQ ID NO:17 Lys Pro Val or Lys Pro.

In one embodiment, when A is helix 2 or helix 7, and C is helix 8, B is Pro, SEQ ID NO:18 Lys Leu Ser Pro Leu, SEQ ID NO:19 Leu Ser Pro Leu, or SEQ ID NO:20 Ser Pro Leu, or a conservative substitution thereof. These amino acids in B may also appear in reverse orientation for example as in SEQ ID NO:21 Leu Pro Ser Leu Lys, SEQ ID NO:22 Leu Pro Ser Leu, and SEQ ID NO:23 Leu Pro Ser.

However, when A is helix 5, helix 6, or helix 8′ and C is helix 8, B is not Pro, SEQ ID NO:18 Lys Leu Ser Pro Leu, SEQ ID NO:19 Leu Ser Pro Leu, SEQ ID NO:20 Ser Pro Leu, SEQ ID NO:21 Leu Pro Ser Leu Lys, SEQ ID NO:22 Leu Pro Ser Leu, or SEQ ID NO:23 Leu Pro Ser or a conservative substitution thereof.

In one embodiment, C is helix 8 of ApoA-I and is SEQ ID NO:24 Leu Glu Ser Phe Lys Val Ser Phe Leu Ser Ala Leu Glu Glu Tyr Thr Lys Lys, or a variation or conservative substitution thereof. These amino acids may also appear in reverse orientation such that Lys is at the N-terminus and Leu is at the C-terminus as SEQ ID NO:25 Lys Lys Thr Tyr Glu Glu Leu Ala Ser Leu Phe Ser Val Lys Phe Ser Glu Leu.

It is to be understood that A and C may be switched in location as in C-B-A. Specific embodiments of peptides represented by generic formula I are:

### Wherein A is Helix 2 or Helix 7 and C is Helix 8

SEQ ID NO:40

Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys;

SEQ ID NO:41

Thr Gln Glu Phe Trp Asp Asn Leu Glu Lys Glu Thr
Glu Gly Leu Arg Gln Glu Leu Val Pro Lys Ala Leu
Glu Ser Phe Lys Val Ser Phe Leu Ser Ala Leu Glu
Glu Tyr Thr Lys Lys;

SEQ ID NO:42

Thr Gln Glu Phe Trp Asp Asn Leu Glu Lys Glu Thr
Glu Gly Leu Arg Gln Glu Lys Pro Val Leu Leu Glu
Ser Phe Lys Val Ser Phe Leu Ser Ala Leu Glu Glu
Tyr Thr Lys Lys;

SEQ ID NO:43

Thr Gln Glu Phe Trp Asp Asn Leu Glu Lys Glu Thr
Glu Gly Leu Arg Gln Glu Pro Val Leu Leu Glu Ser
Phe Lys Val Ser Phe Leu Ser Ala Leu Glu Glu Tyr
Thr Lys Lys

SEQ ID NO:44

Thr Gln Glu Phe Trp Asp Asn Leu Glu Lys Glu Thr
Glu Gly Leu Arg Gln Glu Pro Val Leu Glu Ser Phe
Lys Val Ser Phe Leu Ser Ala Leu Glu Glu Tyr Thr
Lys Lys

SEQ ID NO:45

Thr Gln Glu Phe Trp Asp Asn Leu Glu Lys Glu Thr
Glu Gly Leu Arg Gln Glu Ala Lys Pro Val Leu Glu
Ser Phe Lys Val Ser Phe Leu Ser Ala Leu Glu Glu
Tyr Thr Lys Lys

SEQ ID NO:46

Thr Gln Glu Phe Trp Asp Asn Leu Glu Lys Glu Thr
Glu Gly Leu Arg Gln Glu Ala Lys Pro Leu Glu Ser
Phe Lys Val Ser Phe Leu Ser Ala Leu Glu Glu Tyr
Thr Lys Lys;

SEQ ID NO:47

Thr Gln Glu Phe Trp Asp Asn Leu Glu Lys Glu Thr
Glu Gly Leu Arg Gln Glu Lys Pro Val Leu Glu Ser
Phe Lys Val Ser Phe Leu Ser Ala Leu Glu Glu Tyr
Thr Lys Lys;

SEQ ID NO:48

Thr Gln Glu Phe Trp Asp Asn Leu Glu Lys Glu Thr
Glu Gly Leu Arg Gln Glu Lys Pro Leu Glu Ser Phe
Lys Val Ser Phe Leu Ser Ala Leu Glu Glu Tyr Thr
Lys Lys;

SEQ ID NO:49

Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys;

SEQ ID NO:50

Leu Ala Glu Tyr His Ala Lys Ala Thr Glu His Leu
Ser Thr Leu Ser Glu Lys Leu Val Pro Lys Ala Leu
Glu Ser Phe Lys Val Ser Phe Leu Ser Ala Leu Glu
Glu Tyr Thr Lys Lys;

SEQ ID NO:51

Leu Ala Glu Tyr His Ala Lys Ala Thr Glu His Leu
Ser Thr Leu Ser Glu Lys Lys Pro Val Leu Leu Glu
Ser Phe Lys Val Ser Phe Leu Ser Ala Leu Glu Glu
Tyr Thr Lys Lys

SEQ ID NO:52

Leu Ala Glu Tyr His Ala Lys Ala Thr Glu His Leu
Ser Thr Leu Ser Glu Lys Pro Val Leu Leu Glu Ser
Phe Lys Val Ser Phe Leu Ser Ala Leu Glu Glu Tyr
Thr Lys Lys

SEQ ID NO:53

Leu Ala Glu Tyr His Ala Lys Ala Thr Glu His Leu
Ser Thr Leu Ser Glu Lys Pro Val Leu Glu Ser Phe
Lys Val Ser Phe Leu Ser Ala Leu Glu Glu Tyr Thr
Lys Lys

SEQ ID NO:54

Leu Ala Glu Tyr His Ala Lys Ala Thr Glu His Leu
Ser Thr Leu Ser Glu Lys Ala Lys Pro Val Leu Glu
Ser Phe Lys Val Ser Phe Leu Ser Ala Leu Glu Glu
Tyr Thr Lys Lys

SEQ ID NO:55

Leu Ala Glu Tyr His Ala Lys Ala Thr Glu His Leu
Ser Thr Leu Ser Glu Lys Ala Lys Pro Leu Glu Ser
Phe Lys Val Ser Phe Leu Ser Ala Leu Glu Glu Tyr
Thr Lys Lys

SEQ ID NO:56

Leu Ala Glu Tyr His Ala Lys Ala Thr Glu His Leu
Ser Thr Leu Ser Glu Lys Lys Pro Val Leu Glu Ser
Phe Lys Val Ser Phe Leu Ser Ala Leu Glu Glu Tyr
Thr Lys Lys

SEQ ID NO:57

Leu Ala Glu Tyr His Ala Lys Ala Thr Glu His Leu
Ser Thr Leu Ser Glu Lys Lys Pro Leu Glu Ser Phe
Lys Val Ser Phe Leu Ser Ala Leu Glu Glu Tyr Thr
Lys Lys

SEQ ID NO:58

Thr Gln Glu Phe Trp Asp Asn Leu Glu Lys Glu Thr
Glu Gly Leu Arg Gln Glu Lys Leu Ser Pro Leu Leu
Glu Ser Phe Lys Val Ser Phe Leu Ser Ala Leu Glu
Glu Tyr Thr Lys Lys

SEQ ID NO:59

Thr Gln Glu Phe Trp Asp Asn Leu Glu Lys Glu Thr
Glu Gly Leu Arg Gln Glu Pro Leu Glu Ser Phe Lys
Val Ser Phe Leu Ser Ala Leu Glu Glu Tyr Thr Lys
Lys

SEQ ID NO:60

Thr Gln Glu Phe Trp Asp Asn Leu Glu Lys Glu Thr
Glu Gly Leu Arg Gln Glu Leu Ser Pro Leu Leu Glu
Ser Phe Lys Val Ser Phe Leu Ser Ala Leu Glu Glu
Tyr Thr Lys Lys

SEQ ID NO:61

Thr Gln Glu Phe Trp Asp Asn Leu Glu Lys Glu Thr
Glu Gly Leu Arg Gln Glu Ser Pro Leu Leu Glu Ser
Phe Lys Val Ser Phe Leu Ser Ala Leu Glu Glu Tyr
Thr Lys Lys

SEQ ID NO:62

Thr Gln Glu Phe Trp Asp Asn Leu Glu Lys Glu Thr
Glu Gly Leu Arg Gln Glu Leu Pro Ser Leu Lys Leu
Glu Ser Phe Lys Val Ser Phe Leu Ser Ala Leu Glu
Glu Tyr Thr Lys Lys

SEQ ID NO:63

Thr Gln Glu Phe Trp Asp Asn Leu Glu Lys Glu Thr
Glu Gly Leu Arg Gln Glu Leu Pro Ser Leu Leu Glu
Ser Phe Lys Val Ser Phe Leu Ser Ala Leu Glu Glu
Tyr Thr Lys Lys

SEQ ID NO:64

Thr Gln Glu Phe Trp Asp Asn Leu Glu Lys Glu Thr
Glu Gly Leu Arg Gln Glu Leu Pro Ser Leu Glu Ser
Phe Lys Val Ser Phe Leu Ser Ala Leu Glu Glu Tyr
Thr Lys Lys

SEQ ID NO:65

Leu Ala Glu Tyr His Ala Lys Ala Thr Glu His Leu
Ser Thr Leu Ser Glu Lys Lys Leu Ser Pro Leu Leu
Glu Ser Phe Lys Val Ser Phe Leu Ser Ala Leu Glu
Glu Tyr Thr Lys Lys

SEQ ID NO:66

Leu Ala Glu Tyr His Ala Lys Ala Thr Glu His Leu
Ser Thr Leu Ser Glu Lys Pro Leu Glu Ser Phe Lys
Val Ser Phe Leu Ser Ala Leu Glu Glu Tyr Thr Lys
Lys

SEQ ID NO:67

Leu Ala Glu Tyr His Ala Lys Ala Thr Glu His Leu
Ser Thr Leu Ser Glu Lys Leu Ser Pro Leu Leu Glu
Ser Phe Lys Val Ser Phe Leu Ser Ala Leu Glu Glu
Tyr Thr Lys Lys

SEQ ID NO:68

Leu Ala Glu Tyr His Ala Lys Ala Thr Glu His Leu
Ser Thr Leu Ser Glu Lys Ser Pro Leu Leu Glu Ser
Phe Lys Val Ser Phe Leu Ser Ala Leu Glu Glu Tyr
Thr Lys Lys

SEQ ID NO:69

Leu Ala Glu Tyr His Ala Lys Ala Thr Glu His Leu
Ser Thr Leu Ser Glu Lys Leu Pro Ser Leu Lys Leu
Glu Ser Phe Lys Val Ser Phe Leu Ser Ala Leu Glu
Glu Tyr Thr Lys Lys

SEQ ID NO:70

Leu Ala Glu Tyr His Ala Lys Ala Thr Glu His Leu
Ser Thr Leu Ser Glu Lys Leu Pro Ser Leu Leu Glu
Ser Phe Lys Val Ser Phe Leu Ser Ala Leu Glu Glu
Tyr Thr Lys Lys

SEQ ID NO:71

Leu Ala Glu Tyr His Ala Lys Ala Thr Glu His Leu
Ser Thr Leu Ser Glu Lys Leu Pro Ser Leu Glu Ser
Phe Lys Val Ser Phe Leu Ser Ala Leu Glu Glu Tyr
Thr Lys Lys

### Wherein A is Helix 5 and C is Helix 8

SEQ ID NO:72

Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys;

SEQ ID NO:73

Gly Glu Glu Met Arg Asp Arg Ala Arg Ala His Val
Asp Ala Leu Arg Thr His Leu Val Pro Lys Ala Leu
Glu Ser Phe Lys Val Ser Phe Leu Ser Ala Leu Glu
Glu Tyr Thr Lys Lys;

SEQ ID NO:74

Gly Glu Glu Met Arg Asp Arg Ala Arg Ala His Val
Asp Ala Leu Arg Thr His Lys Pro Val Leu Leu Glu
Ser Phe Lys Val Ser Phe Leu Ser Ala Leu Glu Glu
Tyr Thr Lys Lys

SEQ ID NO:75

Gly Glu Glu Met Arg Asp Arg Ala Arg Ala His Val
Asp Ala Leu Arg Thr His Pro Val Leu Leu Glu Ser
Phe Lys Val Ser Phe Leu Ser Ala Leu Glu Glu Tyr
Thr Lys Lys

SEQ ID NO:76

Gly Glu Glu Met Arg Asp Arg Ala Arg Ala His Val
Asp Ala Leu Arg Thr His Pro Val Leu Glu Ser Phe
Lys Val Ser Phe Leu Ser Ala Leu Glu Glu Tyr Thr
Lys Lys

SEQ ID NO:77

Gly Glu Glu Met Arg Asp Arg Ala Arg Ala His Val
Asp Ala Leu Arg Thr His Ala Lys Pro Val Leu Glu
Ser Phe Lys Val Ser Phe Leu Ser Ala Leu Glu Glu
Tyr Thr Lys Lys

SEQ ID NO:78

Gly Glu Glu Met Arg Asp Arg Ala Arg Ala His Val
Asp Ala Leu Arg Thr His Ala Lys Pro Leu Glu Ser
Phe Lys Val Ser Phe Leu Ser Ala Leu Glu Glu Tyr
Thr Lys Lys

SEQ ID NO:79

Gly Glu Glu Met Arg Asp Arg Ala Arg Ala His Val
Asp Ala Leu Arg Thr His Lys Pro Leu Glu Ser Phe
Lys Val Ser Phe Leu Ser Ala Leu Glu Glu Tyr Thr
Lys Lys

### Wherein A is Helix 6 and C is Helix 8

SEQ ID NO:80

Ser Asp Glu Leu Arg Gln Arg Leu Ala Ala Arg Leu
Glu Ala Leu Lys Glu Asn Ala Lys Pro Val Leu Leu
Glu Ser Phe Lys Val Ser Phe Leu Ser Ala Leu Glu
Glu Tyr Thr Lys Lys;

SEQ ID NO:81

Ser Asp Glu Leu Arg Gln Arg Leu Ala Ala Arg Leu
Glu Ala Leu Lys Glu Asn Leu Val Pro Lys Ala Leu
Glu Ser Phe Lys Val Ser Phe Leu Ser Ala Leu Glu
Glu Tyr Thr Lys Lys;

SEQ ID NO:82

Ser Asp Glu Leu Arg Gln Arg Leu Ala Ala Arg Leu
Glu Ala Leu Lys Glu Asn Lys Pro Val Leu Leu Glu
Ser Phe Lys Val Ser Phe Leu Ser Ala Leu Glu Glu
Tyr Thr Lys Lys;

SEQ ID NO:83

Asp Glu Leu Arg Gln Arg Leu Ala Ala Arg Leu Glu
Ala Leu Lys Glu Asn Pro Val Leu Leu Glu Ser Phe
Lys Val Ser Phe Leu Ser Ala Leu Glu Glu Tyr Thr
Lys Lys;

SEQ ID NO:84

Ser Asp Glu Leu Arg Gln Arg Leu Ala Ala Arg Leu
Glu Ala Leu Lys Glu Asn Pro Val Leu Glu Ser Phe
Lys Val Ser Phe Leu Ser Ala Leu Glu Glu Tyr Thr
Lys Lys;

SEQ ID NO:85

Ser Asp Glu Leu Arg Gln Arg Leu Ala Ala Arg Leu
Glu Ala Leu Lys Glu Asn Ala Lys Pro Val Leu Glu
Ser Phe Lys Val Ser Phe Leu Ser Ala Leu Glu Glu
Tyr Thr Lys Lys;

SEQ ID NO:86

Ser Asp Glu Leu Arg Gln Arg Leu Ala Ala Arg Leu
Glu Ala Leu Lys Glu Asn Ala Lys Pro Leu Glu Ser
Phe Lys Val Ser Phe Leu Ser Ala Leu Glu Glu Tyr
Thr Lys Lys;

SEQ ID NO:87

Ser Asp Glu Leu Arg Gln Arg Leu Ala Ala Arg Leu
Glu Ala Leu Lys Glu Asn Lys Pro Val Leu Glu Ser
Phe Lys Val Ser Phe Leu Ser Ala Leu Glu Glu Tyr
Thr Lys Lys;

SEQ ID NO:88

Ser Asp Glu Leu Arg Gln Arg Leu Ala Ala Arg Leu
Glu Ala Leu Lys Glu Asn Lys Pro Leu Glu Ser Phe
Lys Val Ser Phe Leu Ser Ala Leu Glu Glu Tyr Thr
Lys Lys;

### Wherein A is Helix 8′ and C is Helix 8

SEQ ID NO:89

Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:90

Leu Glu Ser Ala Lys Val Ser Ala Leu Ser Ala Leu
Glu Glu Ala Thr Lys Lys Lys Pro Val Leu Leu Glu
Ser Phe Lys Val Ser Phe Leu Ser Ala Leu Glu Glu
Tyr Thr Lys Lys;

SEQ ID NO:91

Leu Glu Ser Ala Lys Val Ser Ala Leu Ser Ala Leu
Glu Glu Ala Thr Lys Lys Pro Val Leu Leu Glu Ser
Phe Lys Val Ser Phe Leu Ser Ala Leu Glu Glu Tyr
Thr Lys Lys;

SEQ ID NO:92

Leu Glu Ser Ala Lys Val Ser Ala Leu Ser Ala Leu
Glu Glu Ala Thr Lys Lys Pro Val Leu Glu Ser Phe
Lys Val Ser Phe Leu Ser Ala Leu Glu Glu Tyr Thr
Lys Lys;

SEQ ID NO:93

Leu Glu Ser Ala Lys Val Ser Ala Leu Ser Ala Leu
Glu Glu Ala Thr Lys Lys Ala Lys Pro Val Leu Glu
Ser Phe Lys Val Ser Phe Leu Ser Ala Leu Glu Glu
Tyr Thr Lys Lys;

SEQ ID NO:94

Leu Glu Ser Ala Lys Val Ser Ala Leu Ser Ala Leu
Glu Glu Ala Thr Lys Lys Ala Lys Pro Leu Glu Ser
Phe Lys Val Ser Phe Leu Ser Ala Leu Glu Glu Tyr
Thr Lys Lys;

SEQ ID NO:95

Leu Glu Ser Ala Lys Val Ser Ala Leu Ser Ala Leu
Glu Glu Ala Thr Lys Lys Lys Pro Val Leu Glu Ser
Phe Lys Val Ser Phe Leu Ser Ala Leu Glu Glu Tyr
Thr Lys Lys;

SEQ ID NO:96

Leu Glu Ser Ala Lys Val Ser Ala Leu Ser Ala Leu
Glu Glu Ala Thr Lys Lys Lys Pro Leu Glu Ser Phe
Lys Val Ser Phe Leu Ser Ala Leu Glu Glu Tyr Thr
Lys Lys;

SEQ ID NO:97

Leu Glu Ser Ala Lys Val Ser Ala Leu Ser Ala Leu
Glu Glu Ala Thr Lys Lys Ala Lys Pro Val Leu Lys
Lys Thr Tyr Glu Glu Leu Ala Ser Leu Phe Ser Val
Lys Phe Ser Glu Leu;

SEQ ID NO:98

Leu Glu Ser Ala Lys Val Ser Ala Leu Ser Ala Leu
Glu Glu Ala Thr Lys Lys Lys Pro Val Leu Lys Lys
Thr Tyr Glu Glu Leu Ala Ser Leu Phe Ser Val Lys
Phe Ser Glu Leu;

SEQ ID NO:99

Leu Glu Ser Ala Lys Val Ser Ala Leu Ser Ala Leu
Glu Glu Ala Thr Lys Lys Pro Val Leu Lys Lys Thr
Tyr Glu Glu Leu Ala Ser Leu Phe Ser Val Lys Phe
Ser Glu Leu;

SEQ ID NO:100

Leu Glu Ser Ala Lys Val Ser Ala Leu Ser Ala Leu
Glu Glu Ala Thr Lys Lys Pro Val Lys Lys Thr Tyr
Glu Glu Leu Ala Ser Leu Phe Ser Val Lys Phe Ser
Glu Leu;

SEQ ID NO:101

Leu Glu Ser Ala Lys Val Ser Ala Leu Ser Ala Leu
Glu Glu Ala Thr Lys Lys Ala Lys Pro Val Lys Lys
Thr Tyr Glu Glu Leu Ala Ser Leu Phe Ser Val Lys
Phe Ser Glu Leu;

SEQ ID NO:102

Leu Glu Ser Ala Lys Val Ser Ala Leu Ser Ala Leu
Glu Glu Ala Thr Lys Lys Ala Lys Pro Lys Lys Thr
Tyr Glu Glu Leu Ala Ser Leu Phe Ser Val Lys Phe
Ser Glu Leu;

SEQ ID NO:103

Leu Glu Ser Ala Lys Val Ser Ala Leu Ser Ala Leu
Glu Glu Ala Thr Lys Lys Lys Pro Val Lys Lys Thr
Tyr Glu Glu Leu Ala Ser Leu Phe Ser Val Lys Phe
Ser Glu Leu;

SEQ ID NO:104

Leu Glu Ser Ala Lys Val Ser Ala Leu Ser Ala Leu
Glu Glu Ala Thr Lys Lys Lys Pro Lys Lys Thr Tyr
Glu Glu Leu Ala Ser Leu Phe Ser Val Lys Phe Ser
Glu Leu;

SEQ ID NO:105

Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu;

SEQ ID NO:106

Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys;

SEQ ID NO:107

Leu Glu Ser Ala Lys Val Ser Ala Leu Ser Ala Leu
Glu Glu Ala Thr Lys Lys Leu Val Pro Lys Ala Leu
Glu Ser Phe Lys Val Ser Phe Leu Ser Ala Leu Glu
Glu Tyr Thr Lys Lys;

SEQ ID NO:108

Leu Glu Ser Ala Lys Val Ser Ala Leu Ser Ala Leu
Glu Glu Ala Thr Lys Lys Leu Val Pro Lys Ala Lys
Lys Thr Tyr Glu Glu Leu Ala Ser Leu Phe Ser Val
Lys Phe Ser Glu Leu;

SEQ ID NO:109

Lys Lys Thr Ala Glu Glu Leu Ala Ser Leu Ala Ser
Val Lys Ala Ser Glu Leu Leu Val Pro Lys Ala Lys
Lys Thr Tyr Glu Glu Leu Ala Ser Leu Phe Ser Val
Lys Phe Ser Glu Leu;

SEQ ID NO:110

Lys Lys Thr Ala Glu Glu Leu Ala Ser Leu Ala Ser
Val Lys Ala Ser Glu Leu Leu Val Pro Lys Ala Leu
Glu Ser Phe Lys Val Ser Phe Leu Ser Ala Leu Glu
Glu Tyr Thr Lys Lys

In a further embodiment, peptides of the present invention are described by the following subgeneric formula II, in which one or more additional elements indicated as variables D, E, F and W, are added to formula I to make subgeneric formula II.

D-E-(A-B-C)ₙ-F-W II.

(A-B-C)ₙ are as described in formula I above.

D is absent or present and is a peptide as defined in the present specification. In one embodiment, D is a peptide selected from the group consisting of SEQ ID NO:26 Pro Arg Gly Gly Ser Val Leu Val Thr, or multiples, variations or conservative substitutions thereof. These amino acids may also appear in reverse orientation, namely SEQ ID NO:27 Thr Val Leu Val Ser Gly Gly Arg Pro. It is to be understood that one or more of the first six N-terminal amino acids of D, namely SEQ ID NO:28 Pro Arg Gly Gly Ser Val or SEQ ID NO:29 Thr Val Leu Val Ser Gly may occur as D-amino acids;

E is absent or present and is a group linking D and A and is Pro, SEQ ID NO:21 Leu Pro Ser Leu Lys, SEQ ID NO:30 Pro Lys Ala or a conservative substitution thereof, provided that E is present only when D is present. These amino acids may also appear in reverse orientation as in SEQ ID NO:11 Ala Lys Pro Val Leu and SEQ ID NO:16 Ala Lys Pro. When D is absent, E is absent.

F is absent or present and is a group linking C and W and is Pro, SEQ ID NO:31 Ala Leu Ser Pro Leu, SEQ ID NO:11 Ala Lys Pro Val Leu or a conservative substitution thereof, provided that F is present only when W is present. These amino acids may also appear in reverse orientation as in SEQ ID NO:32 Leu Pro Ser Leu Ala and SEQ ID NO:12 Leu Val Pro Lys Ala. When W is absent, F is absent.

W is absent or present and is a peptide as defined in the present specification. In one embodiment, W is a peptide selected from the group consisting of SEQ ID NO:33 Trp Arg Trp Trp Trp Trp), or multiples, variations or conservative substitutions thereof. These amino acids may also appear in reverse orientation, namely SEQ ID NO:34 Trp Trp Trp Trp Arg Trp. It is to be understood that one or more of the amino acids in the W peptide may be D amino acids.

As stated above, in generic formula II, variables D or W may be absent or present. In one embodiment, D is present and W is absent. In another embodiment, W is present and D is absent. In another embodiment, both D and W are present.

Specific embodiments of peptides represented by generic formula II are:

### Wherein A is Helix 2 or Helix 7 and C is Helix 8

SEQ ID NO:111

Pro Arg Gly Gly Ser Val Leu Val Thr Pro Lys Ala
Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:112

Pro Arg Gly Gly Ser Val Leu Val Thr Pro Lys Ala
Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys;

SEQ ID NO:113

Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:114

Pro Arg Gly Gly Ser Val Leu Val Thr Pro Lys Ala
Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:115

Pro Arg Gly Gly Ser Val Leu Val Thr Pro Lys Ala
Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys;

SEQ ID NO:116

Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:117

Pro Arg Gly Gly Ser Val Leu Val Thr Leu Pro Ser
Leu Lys Thr Gln Glu Phe Trp Asp Asn Leu Glu Lys
Glu Thr Glu Gly Leu Arg Gln Glu Lys Leu Ser Pro
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp;

SEQ ID NO:118

Pro Arg Gly Gly Ser Val Leu Val Thr Leu Pro Ser
Leu Lys Thr Gln Glu Phe Trp Asp Asn Leu Glu Lys
Glu Thr Glu Gly Leu Arg Gln Glu Lys Leu Ser Pro
Leu Leu Glu Ser Phe Lys Val Ser Phe Leu Ser Ala
Leu Glu Glu Tyr Thr Lys Lys;

SEQ ID NO:119

Thr Gln Glu Phe Trp Asp Asn Leu Glu Lys Glu Thr
Glu Gly Leu Arg Gln Glu Lys Leu Ser Pro Leu Leu
Glu Ser Phe Lys Val Ser Phe Leu Ser Ala Leu Glu
Glu Tyr Thr Lys Lys Ala Leu Ser Pro Leu Trp Arg
Trp Trp Trp Trp;

SEQ ID NO:120

Pro Arg Gly Gly Ser Val Leu Val Thr Leu Pro Ser
Leu Lys Leu Ala Glu Tyr His Ala Lys Ala Thr Glu
His Leu Ser Thr Leu Ser Glu Lys Lys Leu Ser Pro
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp;

SEQ ID NO:121

Pro Arg Gly Gly Ser Val Leu Val Thr Leu Pro Ser
Leu Lys Leu Ala Glu Tyr His Ala Lys Ala Thr Glu
His Leu Ser Thr Leu Ser Glu Lys Lys Leu Ser Pro
Leu Leu Glu Ser Phe Lys Val Ser Phe Leu Ser Ala
Leu Glu Glu Tyr Thr Lys Lys;
or,

SEQ ID NO:122

Leu Ala Glu Tyr His Ala Lys Ala Thr Glu His Leu
Ser Thr Leu Ser Glu Lys Lys Leu Ser Pro Leu Leu
Glu Ser Phe Lys Val Ser Phe Leu Ser Ala Leu Glu
Glu Tyr Thr Lys Lys Ala Leu Ser Pro Leu Trp Arg
Trp Trp Trp Trp

### Wherein A is Helix 5 or Helix 6 and C is Helix 8

SEQ ID NO:123

Pro Arg Gly Gly Ser Val Leu Val Thr Pro Lys Ala
Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:124

Pro Arg Gly Gly Ser Val Leu Val Thr Pro Lys Ala
Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys;

SEQ ID NO:125

Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:126

Pro Arg Gly Gly Ser Val Leu Val Thr Pro Lys Ala
Ser Asp Glu Leu Arg Gln Arg Leu Ala Ala Arg Leu
Glu Ala Leu Lys Glu Asn Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:127

Pro Arg Gly Gly Ser Val Leu Val Thr Pro Lys Ala
Ser Asp Glu Leu Arg Gln Arg Leu Ala Ala Arg Leu
Glu Ala Leu Lys Glu Asn Ala Lys Pro Val Leu Leu
Glu Ser Phe Lys Val Ser Phe Leu Ser Ala Leu Glu
Glu Tyr Thr Lys Lys;
or,

SEQ ID NO:128

Ser Asp Glu Leu Arg Gln Arg Leu Ala Ala Arg Leu
Glu Ala Leu Lys Glu Asn Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp.

### Wherein A is Helix 8′ and C is Helix 8

SEQ ID NO:129

Pro Arg Gly Gly Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:130

Pro Arg Gly Gly Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:131

Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:132

Pro Arg Gly Gly Ser Val Leu Val Thr Pro Lys Ala
Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:133

Pro Arg Gly Gly Ser Val Leu Val Thr Pro Lys Ala
Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys;

SEQ ID NO:134

Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys Ala Lys Pro Val Leu Trp Arg
Trp Trp Trp Trp;

SEQ ID NO:135

Pro Arg Gly Gly Ser Val Leu Val Thr Pro Lys Ala
Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu Ala Lys Pro Val Leu Trp Arg
Trp Trp Trp Trp;

SEQ ID NO:136

Pro Arg Gly Gly Ser Val Leu Val Thr Pro Lys Ala
Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu;

SEQ ID NO:137

Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu Trp Arg Trp Trp Trp Trp;

SEQ ID NO:138

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Ala Lys Pro Val
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp;

SEQ ID NO:139

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Ala Lys Pro Val
Leu Leu Glu Ser Phe Lys Val Ser Phe Leu Ser Ala
Leu Glu Glu Tyr Thr Lys Lys;

SEQ ID NO:140

Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys Ala Leu Ser Pro Leu Trp Arg
Trp Trp Trp Trp;

SEQ ID NO:141

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Ala Lys Pro Val
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp;

SEQ ID NO:142

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Ala Lys Pro Val
Leu Leu Glu Ser Phe Lys Val Ser Phe Leu Ser Ala
Leu Glu Glu Tyr Thr Lys Lys;

SEQ ID NO:143

Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys Ala Leu Ser Pro Leu Trp Arg
Trp Trp Trp Trp;

SEQ ID NO:144

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Ala Lys Pro Val
Leu Lys Lys Thr Tyr Glu Glu Leu Ala Ser Leu Phe
Ser Val Lys Phe Ser Glu Leu Ala Leu Ser Pro Leu
Trp Arg Trp Trp Trp Trp;

SEQ ID NO:145

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Ala Lys Pro Val
Leu Lys Lys Thr Tyr Glu Glu Leu Ala Ser Leu Phe
Ser Val Lys Phe Ser Glu Leu;

SEQ ID NO:146

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Leu Glu Ser
Phe Lys Val Ser Phe Leu Ser Ala Leu Glu Glu Tyr
Thr Lys Lys Ala Leu Ser Pro Leu Trp Arg Trp Trp
Trp Trp;

SEQ ID NO:147

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Leu Glu Ser
Phe Lys Val Ser Phe Leu Ser Ala Leu Glu Glu Tyr
Thr Lys Lys;

SEQ ID NO:148

Lys Lys Thr Ala Glu Glu Leu Ala Ser Leu Ala Ser
Val Lys Ala Ser Glu Leu Pro Leu Glu Ser Phe Lys
Val Ser Phe Leu Ser Ala Leu Glu Glu Tyr Thr Lys
Lys Ala Leu Ser Pro Leu Trp Arg Trp Trp Trp Trp;

SEQ ID NO:149

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Lys Pro Lys Thr
Tyr Glu Glu Leu Ala Ser Leu Phe Ser Val Lys Phe
Ser Glu Leu Ala Leu Ser Pro Leu Trp Arg Trp Trp
Trp Trp;

SEQ ID NO:150

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Lys Lys Thr
Tyr Glu Glu Leu Ala Ser Leu Phe Ser Val Lys Phe
Ser Glu Leu;

SEQ ID NO:151

Lys Lys Thr Ala Glu Glu Leu Ala Ser Leu Ala Ser
Val Lys Ala Ser Glu Leu Pro Lys Lys Thr Tyr Glu
Glu Leu Ala Ser Leu Phe Ser Val Lys Phe Ser Glu
Leu Trp Arg Trp Trp Trp Trp;

SEQ ID NO:152

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Lys Lys Thr
Tyr Glu Glu Leu Ala Ser Leu Phe Ser Val Lys Phe
Ser Glu Leu Ala Leu Ser Pro Leu Trp Arg Trp Trp
Trp Trp;

SEQ ID NO:153

PRO Arg Gly Gly Ser Val Leu Val Thr Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:154

Pro ARG Gly Gly Ser Val Leu Val Thr Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:155

Pro Arg GLY Gly Ser Val Leu Val Thr Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:156

Pro Arg Gly GLY Ser Val Leu Val Thr Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:157

Pro Arg Gly Gly SER Val Leu Val Thr Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:158

Pro Arg Gly Gly Ser VAL Leu Val Thr Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:159

THR Val Leu Val Ser Gly Gly Arg Pro Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:160

Thr VAL Leu Val Ser Gly Gly Arg Pro Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:161

Thr Val LEU Val Ser Gly Gly Arg Pro Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:162

Thr Val Leu VAL Ser Gly Gly Arg Pro Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:163

Thr Val Leu Val SER Gly Gly Arg Pro Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:164

Thr Val Leu Val Ser GLY Gly Arg Pro Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:165

PRO Arg Gly Gly Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:166

Pro ARG Gly Gly Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:167

Pro Arg GLY Gly Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:168

Pro Arg Gly GLY Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:169

Pro Arg Gly Gly SER Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:170

Pro Arg Gly Gly Ser VAL Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:171

THR Val Leu Val Ser Gly Gly Arg Pro Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:172

Thr VAL Leu Val Ser Gly Gly Arg Pro Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:173

Thr Val LEU Val Ser Gly Gly Arg Pro Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:174

Thr Val Leu VAL Ser Gly Gly Arg Pro Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:175

Thr Val Leu Val SER Gly Gly Arg Pro Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

SEQ ID NO:176

Thr Val Leu Val Ser GLY Gly Arg Pro Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys;

In yet another embodiment, W and D as described in formula III may be switched in location.

W-E-(A-B-C)ₙ-F-D II.

Specific embodiments of peptides represented by generic formula III are as follows:

### Wherein A is Helix 2 or Helix 7 and C is Helix 8

SEQ ID NO:177

Trp Arg Trp Trp Trp Trp Pro Lys Ala Thr Gln Glu
Phe Trp Asp Asn Leu Glu Lys Glu Thr Glu Gly Leu
Arg Gln Glu Ala Lys Pro Val Leu Leu Glu Ser Phe
Lys Val Ser Phe Leu Ser Ala Leu Glu Glu Tyr Thr
Lys Lys Ala Lys Pro Val Leu Pro Arg Gly Gly Ser
Val Leu Val Thr;

SEQ ID NO:178

Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys Ala Lys Pro Val Leu Pro Arg
Gly Gly Ser Val Leu Val Thr;

SEQ ID NO:179

Trp Arg Trp Trp Trp Trp Pro Lys Ala Thr Gln Glu
Phe Trp Asp Asn Leu Glu Lys Glu Thr Glu Gly Leu
Arg Gln Glu Ala Lys Pro Val Leu Leu Glu Ser Phe
Lys Val Ser Phe Leu Ser Ala Leu Glu Glu Tyr Thr
Lys Lys;

SEQ ID NO:180

Trp Arg Trp Trp Trp Trp Pro Lys Ala Leu Ala Glu
Tyr His Ala Lys Ala Thr Glu His Leu Ser Thr Leu
Ser Glu Lys Ala Lys Pro Val Leu Leu Glu Ser Phe
Lys Val Ser Phe Leu Ser Ala Leu Glu Glu Tyr Thr
Lys Lys Ala Lys Pro Val Leu Pro Arg Gly Gly Ser
Val Leu Val Thr;

SEQ ID NO:181

Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys Ala Lys Pro Val Leu Pro Arg
Gly Gly Ser Val Leu Val Thr;

SEQ ID NO:182

Trp Arg Trp Trp Trp Trp Pro Lys Ala Leu Ala Glu
Tyr His Ala Lys Ala Thr Glu His Leu Ser Thr Leu
Ser Glu Lys Ala Lys Pro Val Leu Leu Glu Ser Phe
Lys Val Ser Phe Leu Ser Ala Leu Glu Glu Tyr Thr
Lys Lys;

SEQ ID NO:183

Trp Arg Trp Trp Trp Trp Leu Pro Ser Leu Lys Thr
Gln Glu Phe Trp Asp Asn Leu Glu Lys Glu Thr Glu
Gly Leu Arg Gln Glu Lys Leu Ser Pro Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO:184

Thr Gln Glu Phe Trp Asp Asn Leu Glu Lys Glu Thr
Glu Gly Leu Arg Gln Glu Lys Leu Ser Pro Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

SEQ ID NO:185

Trp Arg Trp Trp Trp Trp Leu Pro Ser Leu Lys Thr
Gln Glu Phe Trp Asp Asn Leu Glu Lys Glu Thr Glu
Gly Leu Arg Gln Glu Lys Leu Ser Pro Leu Leu Glu
Ser Phe Lys Val Ser Phe Leu Ser Ala Leu Glu Glu
Tyr Thr Lys Lys;

SEQ ID NO:186

Trp Arg Trp Trp Trp Trp Leu Pro Ser Leu Lys Leu
Ala Glu Tyr His Ala Lys Ala Thr Glu His Leu Ser
Thr Leu Ser Glu Lys Lys Leu Ser Pro Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO:187

Leu Ala Glu Tyr His Ala Lys Ala Thr Glu His Leu
Ser Thr Leu Ser Glu Lys Lys Leu Ser Pro Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;
or,

SEQ ID NO:188

Trp Arg Trp Trp Trp Trp Leu Pro Ser Leu Lys Leu
Ala Glu Tyr His Ala Lys Ala Thr Glu His Leu Ser
Thr Leu Ser Glu Lys Lys Leu Ser Pro Leu Leu Glu
Ser Phe Lys Val Ser Phe Leu Ser Ala Leu Glu Glu
Tyr Thr Lys Lys.

### Wherein A is Helix 5 or Helix 6 and C is Helix 8

(SEQ ID NO:189)

Trp Arg Trp Trp Trp Trp Pro Lys Ala Gly Glu Glu
Met Arg Asp Arg Ala Arg Ala His Val Asp Ala Leu
Arg Thr His Ala Lys Pro Val Leu Leu Glu Ser Phe
Lys Val Ser Phe Leu Ser Ala Leu Glu Glu Tyr Thr
Lys Lys Ala Lys Pro Val Leu Pro Arg Gly Gly Ser
Val Leu Val Thr;

(SEQ ID NO:190)

Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys Ala Lys Pro Val Leu Pro Arg
Gly Gly Ser Val Leu Val Thr;

(SEQ ID NO:191)

Trp Arg Trp Trp Trp Trp Pro Lys Ala Gly Glu Glu
Met Arg Asp Arg Ala Arg Ala His Val Asp Ala Leu
Arg Thr His Ala Lys Pro Val Leu Leu Glu Ser Phe
Lys Val Ser Phe Leu Ser ALa Leu Glu Glu Tyr Thr
Lys Lys;

(SEQ ID NO:192)

Trp Arg Trp Trp Trp Trp Pro Lys Ala Ser Asp Glu
Ala Ala Arg Leu Glu Ala Leu Lys Asn Ala Lys Pro
Val Leu Leu Gly Ser Phe Lys Val Ser Phe Leu Ser
Ala Leu Glu Glu Tyr Thr Lys Lys Ala Lys Pro Val
Leu Pro Arg Gly Gly Ser Val Leu Val Thr;

(SEQ ID NO:193)

Ser Asp Glu Leu Arg Gln Arf Leu Ala Ala Arg Leu
Glu Ala Ley Lys Glu Asn Ala Lys Pro Val Leu Leu
Glu Ser Phe Lys Val Ser Phe Leu Ser Ala Leu Glu
Glu Tyr Thr Lys Lys Ala Lys Pro Val Leu Pro Arg
Gly Gly Ser Val Leu Val Thr;

(SEQ ID NO:194)

Trp Arg Trp Trp Trp Trp Pro Lys Ala Ser Asp Glu
Leu Arg Gln Arg Leu Ala Ala Arg Leu Glu Ala Leu
Lys Glu Asn Ala Lys Pro Val Leu Leu Glu Ser Phe
Lys Val Ser Phe Leu Ser Ala Leu Glu Glu Tyr Thr
Lys Lys;

(SEQ ID NO:195)

Trp Arg Trp Trp Trp Trp Leu Pro Ser Leu Lys Gly
Glu Glu Met Arg Asp Arg Ala Arg Ala His Val Asp
Ala Leu Arg Thr His Ala Lys Pro Val Leu Leu Glu
Ser Phe Lys Val Ser Phe LEu Ser Ala Leu Glu Glu
Tyr Thr Lys Lys Ala Leu Ser Pro Leu Pro Arg Gly
Gly Ser Val Leu Val Thr;

(SEQ ID NO:196)

Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

(SEQ ID NO:197)

Trp Arg Trp Trp Trp Trp Leu Pro Ser Leu Lys Gly
Glu Glu Met Arg Asp Arg Ala Arg Ala His Val Asp
Ala Leu Arg Thr His Ala Lys Pro Val Leu Leu Glu
Ser Phe Lys Val Ser Phe Leu Ser ALa Leu Glu Glu
Tyr Thr Lys Lys;

(SEQ ID NO:198)

Trp Arg Trp Trp Trp Trp Leu Pro Ser Leu Lys Ser
Asp Glu Leu Arg Gln Arg Leu Ala Ala Arg Leu Glu
ALa Leu Lys Glu Asn Ala Lys Pro Val Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO:199

Ser Asp Glu Leu Arg Gln Arg Leu Ala Ala Arg Leu
Glu Ala Leu Lys Glu Asn Ala Lys Pro Val Leu Leu
Glu Ser Phe Lye Val Ser Phe Leu Ser Ala Leu Glu
Glu Tyr Thr Lys Lys Ala Leu Ser Pro Leu Pro Arg
Gly Gly Ser Val Leu Val Thr;
or

SEQ ID NO:200

Trp Arg Trp Trp Trp Trp Leu Pro Ser Leu Lys Ser
Asp Glu Leu Arg Gln Arg Leu Ala Ala Arg Leu Glu
Ala Leu Lys Glu Asn Ala Lys Pro Val Leu Leu Glu
Ser Phe Lys Val Ser Phe Leu Ser Ala Leu Glu Glu
Tyr Thr Lys Lys.

### Wherein A is Helix 8′ and C is Helix 8

SEQ ID NO:201

Trp Arg Trp Trp Trp Trp Pro Lys Ala Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys Ala Lys Pro Val Leu Pro Arg Gly Gly Ser
Val Leu Val Thr;

SEQ ID NO:202

Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys Ala Lys Pro Val Leu Pro Arg
Gly Gly Ser Val Leu Val Thr;

SEQ ID NO:203

Trp Arg Trp Trp Trp Trp Pro Lys Ala Leu Glu Ser
Ala Lys Val Ser Ala Leu Ser Ala Leu Glu Glu Ala
Thr Lys Lys Ala Lys Pro Val Leu Leu Glu Ser Phe
Lys Val Ser Phe Leu Ser Ala Leu Glu Glu Tyr Thr
Lys Lys;

SEQ ID NO:204

Trp Arg Trp Trp Trp Trp Pro Lys Ala Lys Lys Thr
Ala Glu Glu Leu Ala Ser Leu Ala Ser Val Lys Ala
Ser Glu Leu Ala Lys Pro Val Leu Leu Glu Ser Phe
Lys Val Ser Phe Leu Ser Ala Leu Glu Glu Tyr Thr
Lys Lys Ala Lys Pro Val Leu Pro Arg Gly Gly Ser
Val Leu Val Thr;

SEQ ID NO:205

Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys Ala Lys Pro Val Leu Pro Arg
Gly Gly Ser Val Leu Val Thr;

SEQ ID NO:206

Trp Arg Trp Trp Trp Trp Pro Lys Ala Lys Lys Thr
Ala Glu Glu Leu Ala Ser Leu Ala Ser Val Lys Ala
Ser Glu Leu Ala Lys Pro Val Leu Leu Glu Ser Phe
Lys Val Ser Phe Leu Ser Ala Leu Glu Glu Tyr Thr
Lys Lys;

SEQ ID NO:207

Trp Arg Trp Trp Trp Trp Pro Lys Ala Lys Lys Thr
Ala Glu Glu Leu Ala Ser Leu Ala Ser Val Lys Ala
Ser Glu Leu Ala Lys Pro Val Leu Lys Lys Thr Tyr
Glu Glu Leu Ala Ser Leu Phe Ser Val Lys Phe Ser
Glu Leu Ala Lys Pro Val Leu Pro Arg Gly Gly Ser
Val Leu Val Thr;

SEQ ID NO:208

Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu Ala Lys Pro Val Leu Pro Arg
Gly Gly Ser Val Leu Val Thr;

SEQ ID NO:209

Trp Arg Trp Trp Trp Trp Pro Lys Ala Lys Lys Thr
Ala Glu Glu Leu Ala Ser Leu Ala Ser Val Lys Ala
Ser Glu Leu Ala Lys Pro Val Leu Lys Lys Thr Tyr
Glu Glu Leu Ala Ser Leu Phe Ser Val Lys Phe Ser
Glu Leu;

SEQ ID NO:210

Trp Arg Trp Trp Trp Trp Leu Pro Ser Leu Lys Leu
Glu Ser Ala Lys Val Ser Ala Leu Ser Ala Leu Glu
Glu Ala Thr Lys Lys Ala Lys Pro Val Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO:211

Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

SEQ ID NO:212

Trp Arg Trp Trp Trp Trp Leu Pro Ser Leu Lys Leu
Glu Ser Ala Lys Val Ser Ala Leu Ser Ala Leu Glu
Glu Ala Thr Lys Lys Ala Lys Pro Val Leu Leu Glu
Ser Phe Lys Val Ser Phe Leu Ser Ala Leu Glu Glu
Tyr Thr Lys Lys;

SEQ ID NO:213

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Ala Lys Pro Val Leu Leu Glu
Ser Phe Lys Val Ser Phe Leu Ser Ala Leu Glu Glu
Tyr Thr Lys Lys Ala Leu Ser Pro Leu Pro Arg Gly
Gly Ser Val Leu Val Thr;

SEQ ID NO:214

Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys Ala Leu Ser Pro Leu Pro Arg
Gly Gly Ser Val Leu Val Thr;

SEQ ID NO:215

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Ala Lys Pro Val Leu Leu Glu
Ser Phe Lys Val Ser Phe Leu Ser Ala Leu Glu Glu
Tyr Thr Lys Lys;

SEQ ID NO:216

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Ala Lys Pro Val Leu Lys Lys
Thr Tyr Glu Glu Leu Ala Ser Leu Phe Ser Val Lys
Phe Ser Glu Leu Ala Leu Ser Pro Leu Pro Arg Gly
Gly Ser Val Leu Val Thr;

SEQ ID NO:217

Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu Ala Leu Ser Pro Leu Pro Arg
Gly Gly Ser Val Leu Val Thr;

SEQ ID NO:218

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Ala Lys Pro Val Leu Lys Lys
Thr Tyr Glu Glu Leu Ala Ser Leu Phe Ser Val Lys
Phe Ser Glu Leu;

SEQ ID NO:219

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Leu Glu Ser Phe Lys Val
Ser Phe Leu Ser Ala Leu Glu Glu Tyr Thr Lys Lys
Ala Leu Ser Pro Leu Pro Arg Gly Gly Ser Val Leu
Val Thr;

SEQ ID NO:220

Lys Lys Thr Ala Glu Glu Leu Ala Ser Leu Ala Ser
Val Lys Ala Ser Glu Leu Pro Leu Glu Ser Phe Lys
Val Ser Phe Leu Ser Ala Leu Glu Glu Tyr Thr Lys
Lys Ala Leu Ser Pro Leu Pro Arg Gly Gly Ser Val
Leu Val Thr;

SEQ ID NO:221

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Leu Glu Ser Phe Lys Val
Ser Phe Leu Ser Ala Leu Glu Glu Tyr Thr Lys Lys;

SEQ ID NO:222

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Lys Lys Thr Tyr Glu Glu
Leu Ala Ser Leu Phe Ser Val Lys Phe Ser Glu Leu
Ala Leu Ser Pro Leu Pro Arg Gly Gly Ser Val Leu
Val Thr;

SEQ ID NO:223

Lys Lys Thr Ala Glu Glu Leu Ala Ser Leu Ala Ser
Val Lys Ala Ser Glu Leu Pro Lys Lys Thr Tyr Glu
Glu Leu Ala Ser Leu Phe Ser Val Lys Phe Ser Glu
Leu Ala Leu Ser Pro Leu Pro Arg Gly Gly Ser Val
Leu Val Thr;
and,

SEQ ID NO:224

Trp Arg Trp Trp Trp Trp Leu Pro Ser Leu Lys Lys
Lys Thr Ala Glu Glu Leu Ala Ser Leu Ala Ser Val
Lys Ala Ser Glu Leu Pro Lys Lys Thr Tyr Glu Glu
Leu Ala Ser Leu Phe Ser Val Lys Phe Ser Glu Leu.

In a further embodiment, peptides of the present invention are described by the following subgeneric formula IV, in which one or more additional elements indicated as variables G and H, are added to formula I to make subgeneric formula IV.

G-(A-B-C)ₙ-H IV.

(A-B-C)n are as described in formula I above,

G is absent or present and is a peptide as defined in the present specification. In one embodiment, G is SEQ ID NO:20 Ser Pro Leu or a conservative substitution thereof. These amino acids may also appear in reverse orientation as in SEQ ID NO:23 Leu Pro Ser. It is to be understood that one or more of the amino acids in the G peptide may be D amino acids.

H is absent or present and is a peptide as defined in the present specification. In one embodiment, H is SEQ ID NO:35 Leu Asn Thr Gln or a conservative substitution thereof. These amino acids may also appear in reverse orientation as in SEQ ID NO:36 Gln Thr Asn Leu. It is to be understood that one or more of the amino acids in the H peptide may be D amino acids.

Specific embodiments of peptides represented by generic formula IV are as follows:

### Wherein A is Helix 2 or Helix 7 and C is Helix 8

SEQ ID NO:225

Ser Pro Leu Thr Gln Glu Phe Trp Asp Asn Leu Glu
Lys Glu Thr Glu Gly Leu Arg Gln Glu Ala Lys Pro
Val Leu Leu Glu Ser Phe Lys Val Ser Phe Leu Ser
Ala Leu Glu Glu Tyr Thr Lys Lys Leu Asn Thr Gln;

SEQ ID NO:226

Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:227

Ser Pro Leu Thr Gln Glu Phe Trp Asp Asn Leu Glu
Lys Glu Thr Glu Gly Leu Arg Gln Glu Ala Lys Pro
Val Leu Leu Glu Ser Phe Lys Val Ser Phe Leu Ser
Ala Leu Glu Glu Tyr Thr Lys Lys;

SEQ ID NO:228

Ser Pro Leu Leu Ala Glu Tyr His Ala Lys Ala Thr
Glu His Leu Ser Thr Leu Ser Glu Lys Ala Lys Pro
Val Leu Leu Glu Ser Phe Lys Val Ser Phe Leu Ser
Ala Leu Glu Glu Tyr Thr Lys Lys Leu Asn Thr Gln;

SEQ ID NO:229

Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:230

Ser Pro Leu Leu Ala Glu Tyr His Ala Lys Ala Thr
Glu His Leu Ser Thr Leu Ser Glu Lys Ala Lys Pro
Val Leu Leu Glu Ser Phe Lys Val Ser Phe Leu Ser
Ala Leu Glu Glu Tyr Thr Lys Lys;

SEQ ID NO:231

Ser Pro Leu Thr Gln Glu Phe Trp Asp Asn Leu Glu
Lys Glu Thr Glu Gly Leu Arg Gln Glu Lys Leu Ser
Pro Leu Leu Glu Ser Phe Lys Val Ser Phe Leu Ser
Ala Leu Glu Glu Tyr Thr Lys Lys Leu Asn Thr Gln;

SEQ ID NO:232

Thr Gln Glu Phe Trp Asp Asn Leu Glu Lys Glu Thr
Glu Gly Leu Arg Gln Glu Lys Leu Ser Pro Leu Leu
Glu Ser Phe Lys Val Ser Phe Leu Ser Ala Leu Glu
Glu Tyr Thr Lys Lys Leu Asn Thr Gln;

SEQ ID NO:233

Ser Pro Leu Thr Gln Glu Phe Trp Asp Asn Leu Glu
Lys Glu Thr Glu Gly Leu Arg Gln Glu Lys Leu Ser
Pro Leu Leu Glu Ser Phe Lys Val Ser Phe Leu Ser
Ala Leu Glu Glu Tyr Thr Lys Lys;

SEQ ID NO:234

Ser Pro Leu Leu Ala Glu Tyr His Ala Lys Ala Thr
Glu His Leu Ser Thr Leu Ser Glu Lys Lys Leu Ser
Pro Leu Leu Glu Ser Phe Lys Val Ser Phe Leu Ser
Ala Leu Glu Glu Tyr Thr Lys Lys Leu Asn Thr Gln;

SEQ ID NO:235

Leu Ala Glu Tyr His Ala Lys Ala Thr Glu His Leu
Ser Thr Leu Ser Glu Lys Lys Leu Ser Pro Leu Leu
Glu Ser Phe Lys Val Ser Phe Leu Ser Ala Leu Glu
Glu Tyr Thr Lys Lys Leu Asn Thr Gln;
and,

SEQ ID NO:236

Ser Pro Leu Leu Ala Glu Tyr His Ala Lys Ala Thr
Glu His Leu Ser Thr Leu Ser Glu Lys Lys Leu Ser
Pro Leu Leu Glu Ser Phe Lys Val Ser Phe Leu Ser
Ala Leu Glu Glu Tyr Thr Lys Lys.

### Wherein A is Helix 5 or Helix 6 and C is Helix 8

SEQ ID NO:237

Ser Pro Leu Gly Glu Glu Met Arg Asp Arg Ala Arg
Ala His Val Asp Ala Leu Arg Thr His Ala Lys Pro
Val Leu Leu Glu Ser Phe Lys Val Ser Phe Leu Ser
Ala Leu Glu Glu Tyr Thr Lys Lys Leu Asn Thr Gln;

SEQ ID NO:238

Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:239

Ser Pro Leu Gly Glu Glu Met Arg Asp Arg Ala Arg
Ala His Val Asp Ala Leu Arg Thr His Ala Lys Pro
Val Leu Leu Glu Ser Phe Lys Val Ser Phe Leu Ser
Ala Leu Glu Glu Tyr Thr Lys Lys;

SEQ ID NO:240

Ser Pro Leu Ser Asp Glu Leu Arg Gln Arg Leu Ala
Ala Arg Leu Glu Ala Leu Lys Glu Asn Ala Lys Pro
Val Leu Leu Glu Ser Phe Lys Val Ser Phe Leu Ser
Ala Leu Glu Glu Tyr Thr Lys Lys Leu Asn Thr Gln;

SEQ ID NO:241

Ser Asp Glu Leu Arg Gln Arg Leu Ala Ala Arg Leu
Glu Ala Leu Lys Glu Asn Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:242

Ser Pro Leu Ser Asp Glu Leu Arg Gln Arg Leu Ala
Ala Arg Leu Glu Ala Leu Lys Glu Asn Ala Lys Pro
Val Leu Leu Glu Ser Phe Lys Val Ser Phe Leu Ser
Ala Leu Glu Glu Tyr Thr Lys Lys;

### Wherein A is Helix 8′ and C is Helix 8

SEQ ID NO:243

Ser Pro Leu Leu Glu Ser Ala Lys Val Ser Ala Leu
Ser Ala Leu Glu Glu Ala Thr Lys Lys Ala Lys Pro
Val Leu Leu Glu Ser Phe Lys Val Ser Phe Leu Ser
Ala Leu Glu Glu Tyr Thr Lys Lys Leu Asn Thr Gln;

SEQ ID NO:244

Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:245

Ser Pro Leu Leu Glu Ser Ala Lys Val Ser Ala Leu
Ser Ala Leu Glu Glu Ala Thr Lys Lys Ala Lys Pro
Val Leu Leu Glu Ser Phe Lys Val Ser Phe Leu Ser
Ala Leu Glu Glu Tyr Thr Lys Lys;

SEQ ID NO:246

Ser Pro Leu Lys Lys Thr Ala Glu Glu Leu Ala Ser
Leu Ala Ser Val Lys Ala Ser Glu Leu Ala Lys Pro
Val Leu Leu Glu Ser Phe Lys Val Ser Phe Leu Ser
Ala Leu Glu Glu Tyr Thr Lys Lys Leu Asn Thr Gln;

SEQ ID NO:247

Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:248

Ser Pro Leu Lys Lys Thr Ala Glu Glu Leu Ala Ser
Leu Ala Ser Val Lys Ala Ser Glu Leu Ala Lys Pro
Val Leu Leu Glu Ser Phe Lys Val Ser Phe Leu Ser
Ala Leu Glu Glu Tyr Thr Lys Lys;

SEQ ID NO:249

Ser Pro Leu Lys Lys Thr Ala Glu Glu Leu Ala Ser
Leu Ala Ser Val Lys Ala Ser Glu Leu Ala Lys Pro
Val Leu Lys Lys Thr Tyr Glu Glu Leu Ala Ser Leu
Phe Ser Val Lys Phe Ser Glu Leu Leu Asn Thr Gln;

SEQ ID NO:250

Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu Leu Asn Thr Gln;

SEQ ID NO:251

Ser Pro Leu Lys Lys Thr Ala Glu Glu Leu Ala Ser
Leu Ala Ser Val Lys Ala Ser Glu Leu Ala Lys Pro
Val Leu Lys Lys Thr Tyr Glu Glu Leu Ala Ser Leu
Phe Ser Val Lys Phe Ser Glu Leu;

SEQ ID NO:252

Ser Pro Leu Leu Glu Ser Ala Lys Val Ser Ala Leu
Ser Ala Leu Glu Glu Ala Thr Lys Lys Ala Lys Pro
Val Leu Lys Lys Thr Tyr Glu Glu Leu Ala Ser Leu
Phe Ser Val Lys Phe Ser Glu Leu Leu Asn Thr Gln;

SEQ ID NO:253

Leu Glu Ser Ala Lys Val Ser Ala Leu Ser Ala Leu
Glu Glu Ala Thr Lys Lys Ala Lys Pro Val Leu Lys
Lys Thr Tyr Glu Glu Leu Ala Ser Leu Phe Ser Val
Lys Phe Ser Glu Leu Leu Asn Thr Gln;

SEQ ID NO:254

Ser Pro Leu Leu Glu Ser Ala Lys Val Ser Ala Leu
Ser Ala Leu Glu Glu Ala Thr Lys Lys Ala Lys Pro
Val Leu Lys Lys Thr Tyr Glu Glu Leu Ala Ser Leu
Phe Ser Val Lys Phe Ser Glu Leu;

SEQ ID NO:255

Ser Pro Leu Lys Lys Thr Ala Glu Glu Leu Ala Ser
Leu Ala Ser Val Lys Ala Ser Glu Leu Pro Leu Glu
Ser Phe Lys Val Ser Phe Leu Ser Ala Leu Glu Glu
Tyr Thr Lys Lys Leu Asn Thr Gln;

SEQ ID NO:256

Lys Lys Thr Ala Glu Glu Leu Ala Ser Leu Ala Ser
Val Lys Ala Ser Glu Leu Pro Leu Glu Ser Phe Lys
Val Ser Phe Leu Ser Ala Leu Glu Glu Tyr Thr Lys
Lys Leu Asn Thr Gln;

SEQ ID NO:257

Ser Pro Leu Lys Lys Thr Ala Glu Glu Leu Ala Ser
Leu Ala Ser Val Lys Ala Ser Glu Leu Pro Leu Glu
Ser Phe Lys Val Ser Phe Leu Ser Ala Leu Glu Glu
Tyr Thr Lys Lys;

SEQ ID NO:258

Ser Pro Leu Lys Lys Thr Ala Glu Glu Leu Ala Ser
Leu Ala Ser Val Lys Ala Ser Glu Leu Pro Lys Lys
Thr Tyr Glu Glu Leu Ala Ser Leu Phe Ser Val Lys
Phe Ser Glu Leu Leu Asn Thr Gln;

SEQ ID NO:259

Lys Lys Thr Ala Glu Glu Leu Ala Ser Leu Ala Ser
Val Lys Ala Ser Glu Leu Pro Lys Lys Thr Tyr Glu
Glu Leu Ala Ser Leu Phe Ser Val Lys Phe Ser Glu
Leu Leu Asn Thr Gln;

SEQ ID NO:260

Ser Pro Leu Lys Lys Thr Ala Glu Glu Leu Ala Ser
Leu Ala Ser Val Lys Ala Ser Glu Leu Pro Lys Lys
Thr Tyr Glu Glu Leu Ala Ser Leu Phe Ser Val Lys
Phe Ser Glu Leu;

SEQ ID NO:261

Ser Pro Leu Leu Glu Ser Ala Lys Val Ser Ala Leu
Ser Ala Leu Glu Glu Ala Thr Lys Lys Pro Lys Lys
Thr Tyr Glu Glu Leu Ala Ser Leu Phe Ser Val Lys
Phe Ser.

In a further embodiment, peptides of the present invention are described by the following subgeneric formula V, in which one or more additional elements indicated as variables D, E, F, W, G and H are added to formula I to make subgeneric formula V.

D-E-G-(A-B-C)ₙ-H-F-W V.

(A-B-C)ₙ are as described in formula I above,

D is absent or present and is a peptide as defined in the present specification. In one embodiment, D is a peptide selected from the group consisting of SEQ ID NO:26 Pro Arg Gly Gly Ser Val Leu Val Thr, or multiples, variations or conservative substitutions thereof. These amino acids may also appear in reverse orientation, namely SEQ ID NO:27 Thr Val Leu Val Ser Gly Gly Arg Pro. It is to be understood that one or more of the first six N-terminal amino acids of D, namely SEQ ID NO:28 Pro Arg Gly Gly Ser Val or SEQ ID NO:29 Thr Val Leu Val Ser Gly may occur as D-amino acids;

E is absent or present and is a group linking D and A and is Pro, SEQ ID NO:21 Leu Pro Ser Leu Lys, SEQ ID NO:30 Pro Lys Ala or a conservative substitution thereof, provided that E is present only when D is present. These amino acids may also appear in reverse orientation as in SEQ ID NO:11 Ala Lys Pro Val Leu and SEQ ID NO:16 Ala Lys Pro. When D is absent, E is absent.

F is absent or present and is a group linking C and W and is Pro, SEQ ID NO:31 Ala Leu Ser Pro Leu, SEQ ID NO:11 Ala Lys Pro Val Leu or a conservative substitution thereof, provided that F is present only when W is present. These amino acids may also appear in reverse orientation as in SEQ ID NO:32 Leu Pro Ser Leu Ala and SEQ ID NO:12 Leu Val Pro Lys Ala. When W is absent, F is absent.

W is absent or present and is a peptide as defined in the present specification. In one embodiment, W is a peptide selected from the group consisting of SEQ ID NO:33 Trp Arg Trp Trp Trp Trp, or multiples, variations or conservative substitutions thereof. These amino acids may also appear in reverse orientation, namely SEQ ID NO:34 Trp Trp Trp Trp Arg Trp. It is to be understood that one or more of the amino acids in the W peptide may be D amino acids.

G is absent or present and is a peptide as defined in the present specification provided that G is present when D is absent. In one embodiment, G is SEQ ID NO:20 Ser Pro Leu or a conservative substitution thereof. These amino acids may also appear in reverse orientation as in SEQ ID NO:23 Leu Pro Ser. It is to be understood that one or more of the amino acids in the G peptide may be D amino acids.

H is absent or present and is a peptide as defined in the present specification provided that H is present when W is absent. In one embodiment, H is SEQ ID NO:35 Leu Asn Thr Gln or a conservative substitution thereof. These amino acids may also appear in reverse orientation as in SEQ ID NO:36 Gln Thr Asn Leu. It is to be understood that one or more of the amino acids in the H peptide may be D amino acids.

As stated above, in generic formula V, variables D or W may be absent or present. In one embodiment, D is present and W is absent. In another embodiment, W is present and D is absent. In another embodiment, both D and W are present.

Specific embodiments of peptides represented by generic formula V are:

### Wherein A is Helix 2 or Helix 7 and C is Helix 8

SEQ ID NO:262

Pro Arg Gly Gly Ser Val Leu Val Thr Pro Lys Ala
Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:263

Ser Pro Leu Thr Gln Glu Phe Trp Asp Asn Leu Glu
Lys Glu Thr Glu Gly Leu Arg Gln Glu Ala Lys Pro
Val Leu Leu Glu Ser Phe Lys Val Ser Phe Leu Ser
Ala Leu Glu Glu Tyr Thr Lys Lys Ala Lys Pro Val
Leu Trp Arg Trp Trp Trp Trp;

SEQ ID NO:264

Pro Arg Gly Gly Ser Val Leu Val Thr Pro Lys Ala
Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:265

Ser Pro Leu Leu Ala Glu Tyr His Ala Lys Ala Thr
Glu His Leu Ser Thr Leu Ser Glu Lys Ala Lys Pro
Val Leu Leu Glu Ser Phe Lys Val Ser Phe Leu Ser
Ala Leu Glu Glu Tyr Thr Lys Lys Ala Lys Pro Val
Leu Trp Arg Trp Trp Trp Trp;

SEQ ID NO:266

Pro Arg Gly Gly Ser Val Leu Val Thr Leu Pro Ser
Leu Lys Thr Gln Glu Phe Trp Asp Asn Leu Glu Lys
Glu Thr Glu Gly Leu Arg Gln Glu Lys Leu Ser Pro
Leu Leu Glu Ser Phe Lys Val Ser Phe Leu Ser Ala
Leu Glu Glu Tyr Thr Lys Lys Leu Asn Thr Gln;

SEQ ID NO:267

Ser Pro Leu Thr Gln Glu Phe Trp Asp Asn Leu Glu
Lys Glu Thr Glu Gly Leu Arg Gln Glu Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO:268

Pro Arg Gly Gly Ser Val Leu Val Thr Leu Pro Ser
Leu Lys Leu Ala Glu Tyr His Ala Lys Ala Thr Glu
His Leu Ser Thr Leu Ser Glu Lys Lys Leu Ser Pro
Leu Leu Glu Ser Phe Lys Val Ser Phe Leu Ser Ala
Leu Glu Glu Tyr Thr Lys Lys Leu Asn Thr Gln;
or,

SEQ ID NO:269

Ser Pro Leu Leu Ala Glu Tyr His Ala Lys Ala Thr
Glu His Leu Ser Thr Leu Ser Glu Lys Lys Leu Ser
Pro Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp.

### Wherein A is Helix 5 or Helix 6 and C is Helix 8

SEQ ID NO:270

Pro Arg Gly Gly Ser Val Leu Val Thr Pro Lys Ala
Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:271

Ser Pro Leu Gly Glu Glu Met Arg Asp Arg Ala Arg
Ala His Val Asp Ala Leu Arg Thr His Ala Lys Pro
Val Leu Leu Glu Ser Phe Lys Val Ser Phe Leu Ser
Ala Leu Glu Glu Tyr Thr Lys Lys Ala Lys Pro Val
Leu Trp Arg Trp Trp Trp Trp;

SEQ ID NO:272

Pro Arg Gly Gly Ser Val Leu Val Thr Pro Lys Ala
Ser Asp Glu Leu Arg Gln Arg Leu Ala Ala Arg Leu
Glu Ala Leu Lys Glu Asn Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:273

Ser Pro Leu Ser Asp Glu Leu Arg Gln Arg Leu Ala
Ala Arg Leu Glu Ala Leu Lys Glu Asn Ala Lys Pro
Val Leu Leu Glu Ser Phe Lys Val Ser Phe Leu Ser
Ala Leu Glu Glu Tyr Thr Lys Lys Ala Lys Pro Val
Leu Trp Arg Trp Trp Trp Trp;

SEQ ID NO:274

Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Ala Lys Pro Val
Leu Leu Glu Ser Phe Lys Val Ser Phe Leu Ser Ala
Leu Glu Glu Tyr Thr Lys Lys Leu Asn Thr Gln;

SEQ ID NO:275

Ser Pro Leu Gly Glu Glu Met Arg Asp Arg Ala Arg
Ala His Val Asp Ala Leu Arg Thr His Ala Lys Pro
Val Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO:276

Pro Arg Gly Gly Ser Val Leu Val Thr Leu Pro Ser
Leu Lys Ser Asp Glu Leu Arg Gln Arg Leu Ala Ala
Arg Leu Glu Ala Leu Lys Glu Asn Ala Lys Pro Val
Leu Leu Glu Ser Phe Lys Val Ser Phe Leu Ser Ala
Leu Glu Glu Tyr Thr Lys Lys Leu Asn Thr Gln;

SEQ ID NO:277

Ser Pro Leu Ser Asp Glu Leu Arg Gln Arg Leu Ala
Ala Arg Leu Glu Ala Leu Lys Glu Asn Ala Lys Pro
Val Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp.

### Wherein A is Helix 8′ and C is Helix 8

SEQ ID NO:278

Pro Arg Gly Gly Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:279

Ser Pro Leu Leu Glu Ser Ala Lys Val Ser Ala Leu
Ser Ala Leu Glu Glu Ala Thr Lys Lys Ala Lys Pro
Val Leu Leu Glu Ser Phe Lys Val Ser Phe Leu Ser
Ala Leu Glu Glu Tyr Thr Lys Lys Ala Lys Pro Val
Leu Trp Arg Trp Trp Trp Trp;

SEQ ID NO:280

Pro Arg Gly Gly Ser Val Leu Val Thr Pro Lys Ala
Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys Leu Asn Thr Gln;

SEQ ID NO:281

Ser Pro Leu Lys Lys Thr Ala Glu Glu Leu Ala Ser
Leu Ala Ser Val Lys Ala Ser Glu Leu Ala Lys Pro
Val Leu Leu Glu Ser Phe Lys Val Ser Phe Leu Ser
Ala Leu Glu Glu Tyr Thr Lys Lys Ala Lys Pro Val
Leu Trp Arg Trp Trp Trp Trp;

SEQ ID NO:282

Pro Arg Gly Gly Ser Val Leu Val Thr Pro Lys Ala
Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu Leu Asn Thr Gln;

SEQ ID NO:283

Ser Pro Leu Lys Lys Thr Ala Glu Glu Leu Ala Ser
Leu Ala Ser Val Lys Ala Ser Glu Leu Ala Lys Pro
Val Leu Lys Lys Thr Tyr Glu Glu Leu Ala Ser Leu
Phe Ser Val Lys Phe Ser Glu Leu Ala Lys Pro Val
Leu Trp Arg Trp Trp Trp Trp;

SEQ ID NO:284

Pro Arg Gly Gly Ser Val Leu Val Thr Pro Lys Ala
Leu Glu Ser Ala Lys Val Ser Ala Leu Ser Ala Leu
Glu Glu Ala Thr Lys Lys Ala Lys Pro Val Leu Lys
Lys Thr Tyr Glu Glu Leu Ala Ser Leu Phe Ser Val
Lys Phe Ser Glu Leu Leu Asn Thr Gln;

SEQ ID NO:285

Ser Pro Leu Leu Glu Ser Ala Lys Val Ser Ala Leu
Ser Ala Leu Glu Glu Ala Thr Lys Lys Ala Lys Pro
Val Leu Lys Lys Thr Tyr Glu Glu Leu Ala Ser Leu
Phe Ser Val Lys Phe Ser Glu Leu Ala Lys Pro Val
Leu Trp Arg Trp Trp Trp Trp;

SEQ ID NO:286

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Ala Lys Pro Val
Leu Leu Glu Ser Phe Lys Val Ser Phe Leu Ser Ala
Leu Glu Glu Tyr Thr Lys Lys Leu Asn Thr Gln;

SEQ ID NO:287

Ser Pro Leu Leu Glu Ser Ala Lys Val Ser Ala Leu
Ser Ala Leu Glu Glu Ala Thr Lys Lys Ala Lys Pro
Val Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO:288

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Ala Lys Pro Val
Leu Leu Glu Ser Phe Lys Val Ser Phe Leu Ser Ala
Leu Glu Glu Tyr Thr Lys Lys Leu Asn Thr Gln;

SEQ ID NO:289

Ser Pro Leu Lys Lys Thr Ala Glu Glu Leu Ala Ser
Leu Ala Ser Val Lys Ala Ser Glu Leu Ala Lys Pro
Val Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp;

SEQ ID NO:290

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Ala Lys Pro Val
Leu Lys Lys Thr Tyr Glu Glu Leu Ala Ser Leu Phe
Ser Val Lys Phe Ser Glu Leu Leu Asn Thr Gln;

SEQ ID NO:291

Ser Pro Leu Lys Lys Thr Ala Glu Glu Leu Ala Ser
Leu Ala Ser Val Lys Ala Ser Glu Leu Ala Lys Pro
Val Leu Lys Lys Thr Tyr Glu Glu Leu Ala Ser Leu
Phe Ser Val Lys Phe Ser Glu Leu Ala Leu Ser Pro
Leu Trp Arg Trp Trp Trp Trp;

SEQ ID NO:292

Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Ala Lys Pro Val
Leu Lys Lys Thr Tyr Glu Glu Leu Ala Ser Leu Phe
Ser Val Lys Phe Ser Glu Leu Leu Asn Thr Gln;

SEQ ID NO:293

Ser Pro Leu Leu Glu Ser Ala Lys Val Ser Ala Leu
Ser Ala Leu Glu Glu Ala Thr Lys Lys Ala Lys Pro
Val Leu Lys Lys Thr Tyr Glu Glu Leu Ala Ser Leu
Phe Ser Val Lys Phe Ser Glu Leu Ala Leu Ser Pro
Leu Trp Arg Trp Trp Trp Trp;

SEQ ID NO:294

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Leu Glu Ser
Phe Lys Val Ser Phe Leu Ser Ala Leu Glu Glu Tyr
Thr Lys Lys Leu Asn Thr Gln;

SEQ ID NO:295

Ser Pro Leu Lys Lys Thr Ala Glu Glu Leu Ala Ser
Leu Ala Ser Val Lys Ala Ser Glu Leu Pro Leu Glu
Ser Phe Lys Val Ser Phe Leu Ser Ala Leu Glu Glu
Tyr Thr Lys Lys Ala Leu Ser Pro Leu Trp Arg Trp
Trp Trp Trp;

SEQ ID NO:296

Pro Arg Gly Gly Ser Val Leu Val Thr Leu Pro Ser
Leu Lys Lys Lys Thr Ala Glu Glu Leu Ala Ser Leu
Ala Ser Val Lys Ala Ser Glu Leu Pro Lys Lys Thr
Tyr Glu Glu Leu Ala Ser Leu Phe Ser Val Lys Phe
Ser Glu Leu Leu Asn Thr Gln;

SEQ ID NO:297

Ser Pro Leu Lys Lys Thr Ala Glu Glu Leu Ala Ser
Leu Ala Ser Val Lys Ala Ser Glu Leu Pro Lys Lys
Thr Tyr Glu Glu Leu Ala Ser Leu Phe Ser Val Lys
Phe Ser Glu Leu Ala Leu Ser Pro Leu Trp Arg Trp
Trp Trp Trp.

In a further embodiment, peptides of the present invention are described by formula VI,

D-I-W VI.

D is a peptide as defined in the present specification. In one embodiment, D is a peptide selected from the group consisting of SEQ ID NO:26 Pro Arg Gly Gly Ser Val Leu Val Thr, or multiples, variations or conservative substitutions thereof. These amino acids may also appear in reverse orientation, namely SEQ ID NO:27 Thr Val Leu Val Ser Gly Gly Arg Pro. It is to be understood that one or more of the first six N-terminal amino acids of D, namely SEQ ID NO:28 Pro Arg Gly Gly Ser Val or SEQ ID NO:29 Thr Val Leu Val Ser Gly may occur as D-amino acids;

I is a group linking D and W and is SEQ ID NO:37 Leu Ala Lys Pro Val Leu or a conservative substitution thereof, or multiples thereof or combinations thereof. These amino acids may also appear in reverse orientation, for example, SEQ ID NO:38 Leu Val Pro Lys Ala Leu.

W is absent or present and is a peptide as defined in the present specification. In one embodiment, W is a peptide selected from the group consisting of SEQ ID NO:33 Trp Arg Trp Trp Trp Trp, or multiples, variations or conservative substitutions thereof. These amino acids may also appear in reverse orientation, namely SEQ ID NO:34 Trp Trp Trp Trp Arg Trp. It is to be understood that one or more of the amino acids in the W peptide may be D amino acids.

D and W may also be switched in location in D-I-W to form W-I-D.

Specific embodiments of peptides represented by formula VI are:

SEQ ID NO:298

Pro Arg Gly Gly Ser Val Leu Val Thr Leu Ala Lys
Pro Val Leu Trp Arg Trp Trp Trp Trp;

SEQ ID NO:299

Trp Arg Trp Trp Trp Trp Leu Ala Lys Pro Val Leu
Pro Arg Gly Gly Ser Val Leu Val Thr;

### Substitutions for D

D is a peptide selected from the group consisting of SEQ ID NO:26 Pro Arg Gly Gly Ser Val Leu Val Thr, or multiples, variations or conservative substitutions thereof. These amino acids may also appear in reverse orientation, namely SEQ ID NO:27 Thr Val Leu Val Ser Gly Gly Arg Pro. It is to be understood that one or more of the first six N-terminal amino acids of D, namely SEQ ID NO:28 Pro Arg Gly Gly Ser Val or SEQ ID NO:29 Thr Val Leu Val Ser Gly may occur as D-amino acids.

Conservative amino acid substitutions for amino acids have been described within this application. A few exemplary, non-limiting examples of such substitutions for SEQ ID NO:26 Pro Arg Gly Gly Ser Val Leu Val Thr follow:

P—proline, leucine, valine, isoleucine or methionine;
R—arginine, lysine, valine, leucine, N-nitroarginine, β-cycloarginine, γ-hydroxy-arginine; N-amidinocitruline or 2-amino-4-guanidino-butanoic acid;
G—glycine, serine, alanine, cysteine or threonine;
G—glycine, serine, alanine, cysteine or threonine;
V—valine, leucine, isoleucine or methionine;
L—leucine, isoleucine, methionine or valine;
V—valine, leucine, isoleucine or methionine;
T—threonine, serine, glycine, alanine or cysteine.
Exemplary embodiments for D or include but are not limited to the following:

SEQ ID NO:26

Pro Arg Gly Gly Ser Val Leu Val Thr;

SEQ ID NO:27

Thr Val Leu Val Ser Gly Gly Arg Pro;

SEQ ID NO:300

PRO Arg Gly Gly Ser Val Leu Val Thr;

SEQ ID NO:301

Pro ARG Gly Gly Ser Val Leu Val Thr;

SEQ ID NO:302

Pro Arg GLY Gly Ser Val Leu Val Thr;

SEQ ID NO:303

Pro Arg Gly GLY Ser Val Leu Val Thr;

SEQ ID NO:304

Pro Arg Gly Gly SER Val Leu Val Thr;

SEQ ID NO:305

Pro Arg Gly Gly Ser VAL Leu Val Thr;

SEQ ID NO:306

THR Val Leu Val Ser Gly Gly Arg Pro;

SEQ ID NO:307

Thr VAL Leu Val Ser Gly Gly Arg Pro;

SEQ ID NO:308

Thr Val LEU Val Ser Gly Gly Arg Pro;

SEQ ID NO:309

Thr Val Leu VAL Ser Gly Gly Arg Pro;

SEQ ID NO:310

Thr Val Leu Val SER Gly Gly Arg Pro;

SEQ ID NO:311

Thr Val Leu Val Ser GLY Gly Arg Pro

Substitutions for W

This section discloses an expansion of the peptides in the W domains and other formulae containing W, wherein W is individually absent or present and is a peptide as defined in the present specification. In one embodiment, W is a peptide selected from the group consisting of SEQ ID NO:33 Trp Arg Trp Trp Trp Trp, or multiples, variations or conservative substitutions thereof. These amino acids may also appear in reverse orientation, namely SEQ ID NO:34 Trp Trp Trp Trp Arg Trp. It is to be understood that one or more of the amino acids in the W peptide may be D amino acids. While not wanting to be bound by the following statement, it is believed that W provides an anti-inflammatory component to the peptides of the present invention.

W may be any one of the following peptides: SEQ ID NO:33 Trp Arg Trp Trp Trp Trp; SEQ ID NO:312 Trp Trp Arg Trp Trp Trp; SEQ ID NO:313 Trp Trp Trp Arg Trp Trp; SEQ ID NO:34 Trp Trp Trp Trp Arg Trp; SEQ ID NO:314 Trp Trp Trp Trp Trp Arg; SEQ ID NO:315 Arg Trp Trp Trp Trp Trp, or multiples, variations or combinations thereof, including but not limited to the following,

SEQ ID NO:316

Trp Arg Trp Trp Trp Trp Trp Arg Trp Trp Trp Trp

SEQ ID NO:317

Trp Arg Trp Trp Trp Trp Trp Arg Trp Trp Trp Trp
Trp Arg Trp Trp Trp Trp

SEQ ID NO:318

Trp Trp Arg Trp Trp Trp Trp Trp Arg Trp Trp Trp

SEQ ID NO:319

Trp Trp Arg Trp Trp Trp Trp Trp Arg Trp Trp Trp
Trp Trp Arg Trp Trp Trp

SEQ ID NO:320

Trp Trp Trp Arg Trp Trp Trp Trp Trp Arg Trp Trp

SEQ ID NO:321

Trp Trp Trp Arg Trp Trp Trp Trp Trp Arg Trp Trp
Trp Trp Trp Arg Trp Trp

SEQ ID NO:322

Trp Trp Trp Trp Arg Trp Trp Trp Trp Trp Arg Trp

SEQ ID NO:323

Trp Trp Trp Trp Arg Trp Trp Trp Trp Trp Arg Trp
Trp Trp Trp Trp Arg Trp

SEQ ID NO:324

Trp Trp Trp Trp Trp Arg Trp Trp Trp Trp Trp Arg

SEQ ID NO:325

Trp Trp Trp Trp Trp Arg Trp Trp Trp Trp Trp Arg
Trp Trp Trp Trp Trp Arg

SEQ ID NO:326

Arg Trp Trp Trp Trp Trp Arg Trp Trp Trp Trp Trp

SEQ ID NO:327

Arg Trp Trp Trp Trp Trp Arg Trp Trp Trp Trp Trp
Arg Trp Trp Trp Trp Trp

SEQ ID NO:328

Trp Arg Trp Trp Trp Trp Trp Trp Arg Trp Trp Trp

SEQ ID NO:329

Trp Arg Trp Trp Trp Trp Trp Trp Arg Trp Trp Trp
Trp Trp Trp Arg Trp Trp

SEQ ID NO:330

Trp Arg Trp Trp Trp Trp Trp Trp Trp Trp Arg Trp

SEQ ID NO:331

Trp Arg Trp Trp Trp Trp Trp Trp Trp Trp Arg Trp
Trp Trp Trp Trp Trp Arg.

The present invention includes substitution of amino acids in the peptides listed above. It is to be understood that tryptophan (W) may be conservatively substituted with alanine, phenylalanine, tyrosine or glycine in W. It is also to be understood that arginine (R) may be substituted with lysine, valine or leucine in W.

### Insertion of D Amino Acids in W

Exemplary embodiments include but are not limited to the following:

| | |
|---|---|
| SEQ ID NO:332 | TRP Arg Trp Trp Trp Trp; |
| SEQ ID NO:333 | Trp ARG Trp Trp Trp Trp; |
| SEQ ID NO:334 | Trp Arg TRP Trp Trp Trp; |
| SEQ ID NO:335 | Trp Arg Trp TRP Trp Trp; |
| SEQ ID NO:336 | Trp Arg Trp Trp TRP Trp; |
| SEQ ID NO:337 | Trp Arg Trp Trp Trp TRP; |
| SEQ ID NO:338 | TRP Trp Arg Trp Trp Trp; |
| SEQ ID NO:339 | Trp TRP Arg Trp Trp Trp; |
| SEQ ID NO:340 | Trp Trp ARG Trp Trp Trp; |
| SEQ ID NO:341 | Trp Trp Arg TRP Trp Trp; |
| SEQ ID NO:342 | Trp Trp Arg Trp TRP Trp; |
| SEQ ID NO:343 | Trp Trp Arg Trp Trp TRP; |
| SEQ ID NO:344 | TRP Trp Trp Arg Trp Trp; |
| SEQ ID NO:345 | Trp TRP Trp Arg Trp Trp; |
| SEQ ID NO:346 | Trp Trp TRP Arg Trp Trp; |
| SEQ ID NO:347 | Trp Trp Trp ARG Trp Trp; |
| SEQ ID NO:348 | Trp Trp Trp Arg TRP Trp; |
| SEQ ID NO:349 | Trp Trp Trp Arg Trp TRP. |

### OTHER EXEMPLARY EMBODIMENTS OF THE PRESENT INVENTION

It is to be understood that the letters in the generic formulae I through VI or in components thereof are defined by the text that follows each letter and do not designate an individual amino acid.

It is to be understood that in some embodiments, one or more of the amino acids of the peptides of the present invention are D amino acids. In one embodiment, the N-terminal amino acid, the C-terminal amino acid or both are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention.

N-Terminal Modification and/or C-terminal Modification of the Peptides of the Present Invention

The peptides of the present invention may optionally be acetylated at the N-terminus. The peptides of the present invention may optionally have a carboxy terminal amide. In some embodiments, the peptides of the present invention may have both an acetylated N-terminus and a carboxy terminal amide. Methods of acetylating the N-terminus or adding a carboxy terminal amide are well known to one of ordinary skill in the art. While it is to be understood that any of the peptides disclosed in this application may be modified at the N-terminus, at the C-terminus, or both at the N-terminus and at the C-terminus, the following sequences are presented as exemplary embodiments.

SEQ ID NO:350

Ac-Thr Gln Glu Phe Trp Asp Asn Leu Glu Lys Glu Thr
Glu Gly Leu Arg Gln Glu Ala Lys Pro Val Leu Leu
Glu Ser Phe Lys Val Ser Phe Leu Ser Ala Leu Glu
Glu Tyr Thr Lys Lys-NH₂

SEQ ID NO:351

Ac-Leu Ala Glu Tyr His Ala Lys Ala Thr Glu His Leu
Ser Thr Leu Ser Glu Lys Ala Lys Pro Val Leu Leu
Glu Ser Phe Lys Val Ser Phe Leu Ser Ala Leu Glu
Glu Tyr Thr Lys Lys-NH₂

SEQ ID NO:352

Ac-Thr Gln Glu Phe Trp Asp Asn Leu Glu Lys Glu Thr
Glu Gly Leu Arg Gln Glu Lys Leu Ser Pro Leu Leu
Glu Ser Phe Lys Val Ser Phe Leu Ser Ala Leu Glu
Glu Tyr Thr Lys Lys-NH₂

SEQ ID NO:353

Ac-Leu Ala Glu Tyr His Ala Lys Ala Thr Glu His Leu
Ser Thr Leu Ser Glu Lys Lys Leu Ser Pro Leu Leu
Glu Ser Phe Lys Val Ser Phe Leu Ser Ala Leu Glu
Glu Tyr Thr Lys Lys-NH₂

SEQ ID NO:354

Ac-Gly Glu Glu Met Arg Asp Arg Ala Arg Ala His Val
Asp Ala Leu Arg Thr His Ala Lys Pro Val Leu Leu
Glu Ser Phe Lys Val Ser Phe Leu Ser Ala Leu Glu
Glu Tyr Thr Lys Lys-NH₂

SEQ ID NO:355

Ac-Ser Asp Glu Leu Arg Gln Arg Leu Ala Ala Arg Leu
Glu Ala Leu Lys Glu Asn Ala Lys Pro Val Leu Leu
Glu Ser Phe Lys Val Ser Phe Leu Ser Ala Leu Glu
Glu Tyr Thr Lys Lys-NH₂

SEQ ID NO:356

Ac-Leu Glu Ser Ala Lys Val Ser Ala Leu Ser Ala Leu
Glu Glu Ala Thr Lys Lys Ala Lys Pro Val Leu Lys
Lys Thr Tyr Glu Glu Leu Ala Ser Leu Phe Ser Val
Lys Phe Ser Glu Leu-NH₂

SEQ ID NO:357

Ac-Lys Lys Thr Ala Glu Glu Leu Ala Ser Leu Ala Ser
Val Lys Ala Ser Glu Leu Ala Lys Pro Val Leu Lys
Lys Thr Tyr Glu Glu Leu Ala Ser Leu Phe Ser Val
Lys Phe Ser Glu Leu-NH₂

SEQ ID NO:358

Ac-Lys Lys Thr Ala Glu Glu Leu Ala Ser Leu Ala Ser
Val Lys Ala Ser Glu Leu Ala Lys Pro Val Leu Leu
Glu Ser Phe Lys Val Ser Phe Leu Ser Ala Leu Glu
Glu Tyr Thr Lys Lys-NH₂

SEQ ID NO:359

Ac-Leu Glu Ser Ala Lys Val Ser Ala Leu Ser Ala Leu
Glu Glu Ala Thr Lys Lys Leu Val Pro Lys Ala Leu
Glu Ser Phe Lys Val Ser Phe Leu Ser Ala Leu Glu
Glu Tyr Thr Lys Lys-NH₂

SEQ ID NO:360

Ac-Leu Glu Ser Ala Lys Val Ser Ala Leu Ser Ala Leu
Glu Glu Ala Thr Lys Lys Leu Val Pro Lys Ala Lys
Lys Thr Tyr Glu Glu Leu Ala Ser Leu Phe Ser Val
Lys Phe Ser Glu Leu-NH₂

SEQ ID NO:361

Ac-Lys Lys Thr Ala Glu Glu Leu Ala Ser Leu Ala Ser
Val Lys Ala Ser Glu Leu Leu Val Pro Lys Ala Lys
Lys Thr Tyr Glu Glu Leu Ala Ser Leu Phe Ser Val
Lys Phe Ser Glu Leu-NH₂

SEQ ID NO:362

Ac-Lys Lys Thr Ala Glu Glu Leu Ala Ser Leu Ala Ser
Val Lys Ala Ser Glu Leu Leu Val Pro Lys Ala Leu
Glu Ser Phe Lys Val Ser Phe Leu Ser Ala Leu Glu
Glu Tyr Thr Lys Lys-NH₂

SEQ ID NO:363

Ac-Ser Pro Leu Gly Glu Glu Met Arg Asp Arg Ala Arg
Ala His Val Asp Ala Leu Arg Thr His Ala Lys Pro
Val Leu Leu Glu Ser Phe Lys Val Ser Phe Leu Ser
Ala Leu Glu Glu Tyr Thr Lys Lys Leu Asn Thr Gln-
NH₂;

SEQ ID NO:364

Ac-Ser Pro Leu Ser Asp Glu Leu Arg Gln Arg Leu Ala
Ala Arg Leu Glu Ala Leu Lys Glu Asn Ala Lys Pro
Val Leu Leu Glu Ser Phe Lys Val Ser Phe Leu Ser
Ala Leu Glu Glu Tyr Thr Lys Lys Leu Asn Thr Gln-
NH₂;

SEQ ID NO:365

Ac-Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Ala Lys Pro Val
Leu Leu Glu Ser Phe Ac-Lys Val Ser Phe Leu Ser Ala
Leu Glu Glu Tyr Thr Lys Lys Ala Leu Ser Pro Leu
Trp Arg Trp Trp Trp Trp-NH₂;

SEQ ID NO:366

Ac-Pro Arg Gly Gly Ser Val Leu Val Thr Leu Pro Ser
Leu Lys Gly Glu Glu Met Arg Asp Arg Ala Arg Ala
His Val Asp Ala Leu Arg Thr His Ala Lys Pro Val
Leu Leu Glu Ser Phe Lys Val Ser Phe Leu Ser Ala
Leu Glu Glu Tyr Thr Lys Lys Leu Asn Thr Gln-NH₂;

SEQ ID NO:367

Ac-Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Ala Lys Pro Val
Leu Leu Glu Ser Phe Lys Val Ser Phe Leu Ser Ala
Leu Glu Glu Tyr Thr Lys Lys Ala Leu Ser Pro Leu
Trp Arg Trp Trp Trp Trp-NH₂;

SEQ ID NO:368

Ac-Ser Pro Leu Gly Glu Glu Met Arg Asp Arg Ala Arg
Ala His Val Asp Ala Leu Arg Thr His Ala Lys Pro
Val Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp-NH₂;

SEQ ID NO:369

Ac-Ser Pro Leu Leu Glu Ser Ala Lys Val Ser Ala Leu
Ser Ala Leu Glu Glu Ala Thr Lys Lys Ala Lys Pro
Val Leu Leu Glu Ser Phe Lys Val Ser Phe Leu Ser
Ala Leu Glu Glu Tyr Thr Lys Lys Leu Asn Thr Gln-
NH₂;

SEQ ID NO:370

Ac-Pro Arg Gly Gly Ser Val Leu Val Thr Leu Pro Ser
Leu Lys Leu Glu Ser Ala Lys Val Ser Ala Leu Ser
Ala Leu Glu Glu Ala Thr Lys Lys Ala Lys Pro Val
Leu Leu Glu Ser Phe Lys Val Ser Phe Leu Ser Ala
Leu Glu Glu Tyr Thr Lys Lys Leu Asn Thr Gln-NH₂;

SEQ ID NO:371

Ac-Ser Pro Leu Leu Glu Ser Ala Lys Val Ser Ala Leu
Ser Ala Leu Glu Glu Ala Thr Lys Lys Ala Lys Pro
Val Leu Leu Glu Ser Phe Lys Val Ser Phe Leu Ser
Ala Leu Glu Glu Tyr Thr Lys Lys Ala Leu Ser Pro
Leu Trp Arg Trp Trp Trp Trp-NH₂;

SEQ ID NO:372

Ac-Leu Glu Ser Ala Lys Val Ser Ala Leu Ser Ala Leu
Glu Glu Ala Thr Lys Lys Ala Lys Pro Val Leu Leu
Glu Ser Phe Lys Val Ser Phe Leu Ser Ala Leu Glu
Glu Tyr Thr Lys Lys-NH₂.

### Modified Peptides of the Present Invention

The present invention may be used for the production of the peptides or peptide analogs of the present invention. “Proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the protein, or to the alpha-amino group (imino group when participating in a peptide bond) of an amino acid at any other location within the protein. Similarly, the term “carboxy terminus” (abbreviated C-terminus) refers to the free carboxyl group on the amino acid at the carboxy terminus of a protein, or to the carboxyl group of an amino acid at any other location within the protein.

Typically, the amino acids making up a protein are numbered in order, starting at the amino terminal and increasing in the direction toward the carboxy terminal of the protein. Thus, when one amino acid is said to “follow” another, that amino acid is positioned closer to the carboxy terminal of the protein than the preceding amino acid.

The term “residue” is used herein to refer to an amino acid (D or L) or an amino acid mimetic that is incorporated into a protein by an amide bond. When a D amino acid is present in the peptides of the present invention, the three letter designation for the amino acid appears in upper case instead of a capital letter. For example the amino acid serine, represented as Ser indicates an L amino acid. The D amino acid form is represented as the upper case letters SER. This is not to be confused with letters appearing as subscripts used in generic formula and defined as variables herein. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art. It is to be understood that in some embodiments, one or more of the amino acids of the peptides of the present invention are D amino acids. In one embodiment, the N-terminal amino acid, the C-terminal amino acid, or both, are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention.

Furthermore, one of skill will recognize that, as mentioned above, individual substitutions, deletions or additions which alter, add or delete a single amino acid or a small percentage of amino acids (typically less than about 5%, or typically less than about 1%) in a sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q), Histidine (H);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

A conservative substitution is a substitution in which the substituting amino acid (naturally occurring or modified) is structurally related to the amino acid being substituted, i.e., has about the same size and electronic properties as the amino acid being substituted. Thus, the substituting amino acid would have the same or a similar functional group in the side chain as the original amino acid. A “conservative substitution” also refers to utilizing a substituting amino acid which is identical to the amino acid being substituted except that a functional group in the side chain is protected with a suitable protecting group. Peptides of the present invention include conservatively substituted peptides, wherein these conservative substitutions occur at 1%, 3%, 5%, 7%, 10%, 15%, 20%, 25%, 30%, 40%, or 50% of the amino acid residues. Peptides of the present invention include peptides that are homologous at 50%, 60%, 70%, 80%, 90%, 95%, 97%, 98%, 99% of the entire sequence of the peptide.

Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those which facilitate transport of the peptide through membranes, for example, by reducing the hydrophilicity and increasing the lipophilicity of the peptide, and which can be cleaved, either by hydrolysis or enzymatically (Ditter et al., 1968. J. Pharm. Sci. 57:783; Ditter et al., 1968. J. Pharm. Sci. 57:828; Ditter et al., 1969. J. Pharm. Sci. 58:557; King et al., 1987. Biochemistry 26:2294; Lindberg et al., 1989. Drug Metabolism and Disposition 17:311; Tunek et al., 1988. Biochem. Pharm. 37:3867; Anderson et al., 1985 Arch. Biochem. Biophys. 239:538; and Singhal et al., 1987. FASEB J. 1:220). Suitable hydroxyl protecting groups include ester, carbonate and carbamate protecting groups. Suitable amine protecting groups include acyl groups and alkoxy or aryloxy carbonyl groups, as described above for N-terminal protecting groups. Suitable carboxylic acid protecting groups include aliphatic, benzyl and aryl esters, as described below for C-terminal protecting groups. In one embodiment, the carboxylic acid group in the side chain of one or more glutamic acid or aspartic acid residues in a peptide of the present invention is protected, preferably as a methyl, ethyl, benzyl or substituted benzyl ester, more preferably as a benzyl ester. Provided below are groups of naturally occurring and modified amino acids in which each amino acid in a group has similar electronic and steric properties. Thus, a conservative substitution can be made by substituting an amino acid with another amino acid from the same group. It is to be understood that these groups are non-limiting, i.e. that there are additional modified amino acids which could be included in each group.
Group I includes leucine, isoleucine, valine, methionine and modified amino acids having the following side chains: ethyl, n-propyl n-butyl. Preferably, Group I includes leucine, isoleucine, valine and methionine.
Group II includes glycine, alanine, valine and a modified amino acid having an ethyl side chain. Preferably, Group II includes glycine and alanine.
Group III includes phenylalanine, phenylglycine, tyrosine, tryptophan, cyclohexylmethyl glycine, and modified amino residues having substituted benzyl or phenyl side chains. Preferred substituents include one or more of the following: halogen, methyl, ethyl, nitro, —NH₂, methoxy, ethoxy and —CN. Preferably, Group III includes phenylalanine, tyrosine and tryptophan.
Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, —CO—NH— alkylated glutamine or asparagines (e.g., methyl, ethyl, n-propyl and iso-propyl) and modified amino acids having the side chain-(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, methyl aspartate, ethyl aspartate, benzyl aspartate and methyl glutamate, ethyl glutamate and benzyl glutamate, glutamine and asparagine.
Group V includes histidine, lysine, ornithine, arginine, N-nitroarginine, β-cycloarginine, γ-hydroxyarginine, N-amidinocitruline and 2-amino-4-guanidinobutanoic acid, homologs of lysine, homologs of arginine and homologs of ornithine. Preferably, Group V includes histidine, lysine, arginine and ornithine. A homolog of an amino acid includes from 1 to about 3 additional or subtracted methylene units in the side chain.
Group VI includes serine, threonine, and modified amino acids having C1-C5 straight or branched alkyl side chains substituted with —OH or —SH, for example, —CH₂CH₂OH, —CH₂CH₂CH₂OH or —CH₂CH₂OHCH₃. Preferably, Group VI includes serine, or threonine.

In another aspect, suitable substitutions for amino acid residues include “severe” substitutions. A “severe substitution” is a substitution in which the substituting amino acid (naturally occurring or modified) has significantly different size and/or electronic properties compared with the amino acid being substituted. Thus, the side chain of the substituting amino acid can be significantly larger (or smaller) than the side chain of the amino acid being substituted and/or can have functional groups with significantly different electronic properties than the amino acid being substituted. Examples of severe substitutions of this type include the substitution of phenylalanine or cyclohexylmethyl glycine for alanine, isoleucine for glycine, a D amino acid for the corresponding L amino acid, or —NH—CH[(—CH₂)₅—COOH]—CO— for aspartic acid. Alternatively, a functional group may be added to the side chain, deleted from the side chain or exchanged with another functional group. Examples of severe substitutions of this type include adding of valine, leucine or isoleucine, exchanging the carboxylic acid in the side chain of aspartic acid or glutamic acid with an amine, or deleting the amine group in the side chain of lysine or ornithine. In yet another alternative, the side chain of the substituting amino acid can have significantly different steric and electronic properties that the functional group of the amino acid being substituted. Examples of such modifications include tryptophan for glycine, lysine for aspartic acid and —(CH₂)₄COOH for the side chain of serine. These examples are not meant to be limiting.

In addition to the naturally occurring genetically encoded amino acids, amino acid residues in the peptides may be substituted with naturally occurring non-encoded amino acids and synthetic amino acids. Certain commonly encountered amino acids which provide useful substitutions include, but are not limited to, β-alanine and other omega-amino acids, such as 3-aminopropionic acid, 2,3-diaminopropionic acid, 4-aminobutyric acid and the like; α-aminoisobutyric acid; ε-aminohexanoic acid; δ-aminovaleric acid; N-methylglycine or sarcosine; ornithine; citrulline; t-butylalanine; t-butylglycine; N-methylisoleucine; phenylglycine; cyclohexylalanine; norleucine; naphthylalanine; 4-chlorophenylalanine; 2-fluorophenylalanine; 3-fluorophenylalanine; 4-fluorophenylalanine; penicillamine; 1,2,3,4-tetrahydroisoquinoline-3-carboxylic acid; β2-thienylalanine; methionine sulfoxide; homoarginine; N-acetyl lysine; 2,4-diaminobutyric acid; 2,3-diaminobutyric acid; p-aminophenylalanine; N-methyl valine; homocysteine; homophenylalanine; homoserine; hydroxyproline; homoproline; N-methylated amino acids; and peptoids (N-substituted glycines).

While in certain embodiments, the amino acids of the peptides will be substituted with L-amino acids, the substitutions are not limited to L-amino acids. Thus, also encompassed by the present disclosure are modified forms of the peptides, wherein an L-amino acid is replaced with an identical D-amino acid (e.g., L-Arg→D-Arg) or with a conservatively-substituted D-amino acid (e.g., LArg→D-Lys), and vice versa.

Additional aspects of the disclosure include analogs, variants, derivatives, and mimetics based on the amino acid sequence of the peptides disclosed herein. Typically, mimetic compounds are synthetic compounds having a three-dimensional structure (of at least part of the mimetic compound) that mimics, for example, the primary, secondary, and/or tertiary structural, and/or electrochemical characteristics of a selected peptide, structural domain, active site, or binding region (e.g., a homotypic or heterotypic binding site, a catalytic active site or domain, a receptor or ligand binding interface or domain, or a structural motif) thereof. The mimetic compound will often share a desired biological activity with a native peptide, as discussed herein (e.g., the ability to interact with lipids). Typically, at least one subject biological activity of the mimetic compound is not substantially reduced in comparison to, and is often the same as or greater than, the activity of the native peptide on which the mimetic was modeled.

A variety of techniques well known to one of skill in the art are available for constructing synthetic peptide mimetics with the same, similar, increased, or reduced biological activity as the corresponding native peptide. Often these analogs, variants, derivatives and mimetics will exhibit one or more desired activities that are distinct or improved from the corresponding native peptide, for example, improved characteristics of solubility, stability, lipid interaction, and/or susceptibility to hydrolysis or proteolysis (see, e.g., Morgan and Gainor, Ann. Rep. Med. Chem. 24:243-252, 1989). In addition, mimetic compounds of the disclosure can have other desired characteristics that enhance their therapeutic application, such as increased cell permeability, greater affinity and/or avidity for a binding partner, and/or prolonged biological half-life. The mimetic compounds of the disclosure can have a backbone that is partially or completely non-peptide, but with side groups identical to the side groups of the amino acid residues that occur in the peptide on which the mimetic compound is modeled. Several types of chemical bonds, for example, ester, thioester, thioamide, retroamide, reduced carbonyl, dimethylene and ketomethylene bonds, are known in the art to be generally useful substitutes for peptide bonds in the construction of protease-resistant mimetic compounds.

In one embodiment, peptides useful within the disclosure are modified to produce synthetic peptide mimetics by replacement of one or more naturally occurring side chains of the 20 genetically encoded amino acids (or D-amino acids) with other side chains, for example with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from a 5-membered ring to a 4-, 6-, or 7-membered ring. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and thiazolyl groups. These heterocyclic groups can be substituted or unsubstituted. Where a group is substituted, the substituent can be alkyl, alkoxy, halogen, oxygen, or substituted or unsubstituted phenyl. Peptides, as well as peptide analogs and mimetics, can also be covalently bound to one or more of a variety of nonproteinaceous polymers, for example, polyethylene glycol, polypropylene glycol, or polyoxyalkenes, as described in U.S. Pat. Nos. 4,640,835; 4,496,689; 4,301,144; 4,670,417; 4,791,192; and 4,179,337.

Other peptide analogs and mimetics within the scope of the disclosure include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues (e.g., lysine or arginine). Acyl groups are selected from the group of alkyl-moieties including C3 to C18 alkyl, thereby forming alkanoyl aroyl species. Also embraced are versions of a native primary amino acid sequence which have other minor modifications, including phosphorylated amino acid residues, for example, phosphotyrosine, phosphoserine, or phosphothreonine, or other moieties, including ribosyl groups or cross-linking reagents.

In the peptides disclosed herein, the linkage between amino acid residues can be a peptide bond or amide linkage (e.g., —C—C(O)NH—). Alternatively, one or more amide linkages is optionally replaced with a linkage other than amide, for example, a substituted amide. Substituted amides generally include, but are not limited to, groups of the formula —C(O)NR—, where R is (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl, and substituted 6-26 membered alkheteroaryl. Additionally, one or more amide linkages can be replaced with peptidomimetic or amide mimetic moieties which do not significantly interfere with the structure or activity of the peptides. Suitable amide mimetic moieties are described, for example, in Olson et al., J. Med. Chem. 36:3039-3049, 1993.

The peptides of the present invention may optionally be acetylated at the N-terminus. The peptides of the present invention may optionally have a carboxy terminal amide. In some embodiments, the peptides of the present invention may have both an acetylated N-terminus and a carboxy terminal amide. Methods of acetylating the N-terminus or adding a carboxy terminal amide are well known to one of ordinary skill in the art.

### IV. Overview of Several Embodiments

Isolated peptides and peptide analogs with domains that promote lipid efflux from cells are disclosed herein. The isolated peptides and peptide analogs are believed to stimulate LCAT activity. In some embodiments, the isolated peptides and peptide analogs of the present invention contain domains that promote lipid efflux and also possess anti-inflammatory activity, for example the A and C domains in the ABC peptides. Isolated peptides and peptide analogs that also include an additional functional domain or peptide are also disclosed herein. This additional functional domain provides anti-inflammatory biological activity, especially with regard to the domains indicated by W and/or D. This additional anti-inflammatory domain, or the domains that possess both lipid efflux and anti-inflammatory activity, provide additional benefit as many vascular conditions are considered by one of ordinary skill in the art to have inflammation as a component of the disease etiology.

For administration to an animal or a human, the peptides and peptide analogs of the present invention are combined with an acceptable carrier to form a pharmaceutical composition and are administered to the animal or the human.

In another embodiment, a method is provided for treating or inhibiting dyslipidemic and vascular disorders in an animal or a human. This method includes administering to the animal or the human a therapeutically effective amount of a pharmaceutical composition that includes one or more isolated peptides or peptide analogs and one or more anti-inflammatory domains. In specific, non-limiting examples, the dyslipidemic and vascular disorders include hyperlipidemia, hyperlipoproteinemia, hypercholesterolemia, hypertriglyceridemia, HDL deficiency, apoA-I deficiency, coronary artery disease, atherosclerosis, myocardial infarction, stroke, thrombotic stroke, peripheral vascular disease, restenosis, acute coronary syndrome, and reperfusion myocardial injury. In yet another specific example of the provided method, the isolated peptide includes two domains, one or more anti-inflammatory domains (D and W) and has an amino acid sequence as set forth herein. In yet another specific example of the provided method, the isolated peptide includes a domain or domains (A and C) that possess both anti-inflammatory and lipid efflux activity and has an amino acid sequence as set forth herein.

Additionally, in representative peptides disclosed herein, the amino- and carboxy-terminal ends can be modified by conjugation with various functional groups. Neutralization of the terminal charge of synthetic peptide mimetics of apolipoproteins has been shown to increase their lipid affinity (Yancey et al., Biochem. 34:7955-7965, 1995; Venkatachalapathi et al., Protein: Structure, Function and Genetics 15:349-359, 1993). For example, acetylation of the amino terminal end of amphipathic peptides increases the lipid affinity of the peptide (Mishra et al., J. Biol. Chem. 269:7185-7191, 1994). Other possible end modifications are described, for example, in Brouillette et al., Biochem. Biophys. Acta 1256:103-129, 1995: Mishra et al., J. Biol. Chem. 269:7185-7191, 1994; and Mishra et al., J. Biol. Chem. 270:1602-1611, 1995.

In another embodiment, a detectable moiety can be linked to any of the peptides disclosed herein, creating a peptide-detectable moiety conjugate. The peptides or peptide analogs disclosed herein may be labeled using labels and techniques known to one of ordinary skill in the art. Some of these labels are described in the “Handbook of Fluorescent Probes and Research Products”, ninth edition, Richard P. Haugland (ed) Molecular Probes, Inc. Eugene, Oreg.), which is incorporated herein in its entirety. Detectable moieties suitable for such use include any composition detectable by spectroscopic, photochemical, biochemical, immunochemical, electrical, optical, magnetic or chemical means. The detectable moieties contemplated for the present disclosure can include, but are not limited to, an immunofluorescent moiety (e.g., fluorescein, rhodamine, Texas red, and the like), a radioactive moiety (e.g., ³H, ³²P, ¹²⁵I, ¹³¹I, ³⁵S), an enzyme moiety (e.g., horseradish peroxidase, alkaline phosphatase), a calorimetric moiety (e.g., colloidal gold, biotin, colored glass or plastic, and the like).

The detectable moiety can be liked to the peptide or peptide analog at either the N- and/or C-terminus. Optionally, a linker can be included between the peptide or peptide analog and the detectable moiety.

The detectable peptides of the present invention may be employed in imaging techniques to identify sites of atherosclerotic plaque and sites of cholesterol efflux. Such imaging techniques may occur in vivo using IVUS, NMR, CAT, PET or other techniques commonly known to one of ordinary skill in the art.

Means of detecting such moieties are well known to those of skill in the art. Thus, for example, radiolabels may be detected using photographic film, gamma counters or scintillation counters. Fluorescent markers may be detected using a photodetector to detect emitted illumination. Enzymatic labels are typically detected by providing the enzyme with a substrate and detecting the reaction product produced by the action of the enzyme on the substrate, and calorimetric labels are detected by simply visualizing the colored label.

The linkers contemplated by the present disclosure can be any bifunctional molecule capable of covalently linking two peptides to one another. Thus, suitable linkers are bifunctional molecules in which the functional groups are capable of being covalently attached to the N- and/or C-terminus of a peptide. Functional groups suitable for attachment to the N- or C-terminus of peptides are well known in the art, as are suitable chemistries for effecting such covalent bond formation.

The linker may be flexible, rigid or semi-rigid. Suitable linkers include, for example, amino acid residues such as Pro or Gly or peptide segments containing from about 2 to about 5, 10, 15, 20, or even more amino acids, bifunctional organic compounds such as H₂N(CH₂)ₙCOOH where n is an integer from 1 to 12, and the like. Examples of such linkers, as well as methods of making such linkers and peptides incorporating such linkers, are well-known in the art (see, e.g., Hunig et al., Chem. Ber. 100:3039-3044, 1974 and Basak et al., Bioconjug. Chem. 5:301-305, 1994).

Conjugation methods applicable to the present disclosure include, by way of non-limiting example, reductive amination, diazo coupling, thioether bond, disulfide-bond, amidation and thiocarbamoyl chemistries. In one embodiment, the amphipathic α-helical domains are “activated” prior to conjugation. Activation provides the necessary chemical groups for the conjugation reaction to occur. In one specific, non-limiting example, the activation step includes derivatization with adipic acid dihydrazide. In another specific, non-limiting example, the activation step includes derivatization with the N-hydroxysuccinimide ester of 3-(2-pyridyl dithio)-propionic acid. In yet another specific, non-limiting example, the activation step includes derivatization with succinimidyl 3-(bromoacetamido) propionate. Further, non-limiting examples of derivatizing agents include succinimidylformylbenzoate and succinimidyllevulinate.

### V. Synthesis and Purification of the Peptides

The peptides or peptide analogs of the disclosure can be prepared using virtually any technique known to one of ordinary skill in the art for the preparation of peptides.

For example, the peptides can be prepared using step-wise solution or solid phase peptide syntheses, or recombinant DNA techniques, or the equivalents thereof

A. Chemical Synthesis

Peptides of the disclosure containing amino acids having either the D- or L-configuration can be readily synthesized by automated solid phase procedures well known in the art. Suitable syntheses can be performed by utilizing “T-boc” or “F-moc” procedures. Techniques and procedures for solid phase synthesis are described in Solid Phase Peptide Synthesis: A Practical Approach, by E. Atherton and R. C. Sheppard, published by IRL, Oxford University Press, 1989. Alternatively, the peptides may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). This is particularly the case with glycine containing peptides. Other methods useful for synthesizing the peptides of the disclosure are described in Nakagawa et al., J. Am. Chem. Soc. 107:7087-7092, 1985.

Additional exemplary techniques known to those of ordinary skill in the art of peptide and peptide analog synthesis are taught by Bodanszky, M. and Bodanszky, A., The Practice of Peptide Synthesis, Springer Verlag, New York, 1994; and by Jones, J., Amino Acid and Peptide Synthesis, 2nd ed., Oxford University Press, 2002. The Bodanszky and Jones references detail the parameters and techniques for activating and coupling amino acids and amino acid derivatives. Moreover, the references teach how to select, use and remove various useful functional and protecting groups.

Peptides of the disclosure having either the D- or L-configuration can also be readily purchased from commercial suppliers of synthetic peptides. Such suppliers include, for example, Advanced ChemTech (Louisville, Ky.), Applied Biosystems (Foster City, Calif.), Anaspec (San Jose, Calif.), and Cell Essentials (Boston, Mass.).

B. Recombinant Synthesis

If the peptide is composed entirely of gene-encoded amino acids, or a portion of it is so composed, the peptide or the relevant portion can also be synthesized using conventional recombinant genetic engineering techniques. For recombinant production, a polynucleotide sequence encoding the peptide is inserted into an appropriate expression vehicle, that is, a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Sambrook et al. (ed.), Molecular Cloning: A Laboratory Manual, 2^{nd} ed., vol. 1-3, Cold Spring Harbor Laboratory Press, Cold Spring Harbor, N.Y., 1989, Ch. 17 and Ausubel et al. Short Protocols in Molecular Biology, 4^{th} ed., John Wiley & Sons, Inc., 1999).

To increase efficiency of production, the polynucleotide can be designed to encode multiple units of the peptide separated by enzymatic cleavage sites. The resulting polypeptide can be cleaved (e.g., by treatment with the appropriate enzyme) in order to recover the peptide units. This can increase the yield of peptides driven by a single promoter. In one embodiment, a polycistronic polynucleotide can be designed so that a single mRNA is transcribed which encodes multiple peptides, each coding region operatively linked to a cap-independent translation control sequence, for example, an internal ribosome entry site (IRES). When used in appropriate viral expression systems, the translation of each peptide encoded by the mRNA is directed internally in the transcript, for example, by the IRES. Thus, the polycistronic construct directs the transcription of a single, large polycistronic mRNA which, in turn, directs the translation of multiple, individual peptides. This approach eliminates the production and enzymatic processing of polyproteins and can significantly increase yield of peptide driven by a single promoter.

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus (CaMV) or tobacco mosaic virus (TMV)) or transformed with recombinant plasmid expression vectors (e.g., Ti plasmid) containing an appropriate coding sequence; plant systems (Arabidopsis) for expression of Apo AI avaliable from SemBioSys Genetics Inc. (Calgary, Canada); or animal cell systems.

The expression elements of the expression systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage h., plac, ptrp, ptac (ptrp-lac hybrid promoter) and the like can be used. When cloning in insect cell systems, promoters such as the baculovirus polyhedron promoter can be used. When cloning in plant cell systems, promoters derived from the genome of plant cells (e.g., heat shock promoters, the promoter for the small subunit of RUBISCO, the promoter for the chlorophyll a/b binding protein) or from plant viruses (e.g., the 35S RNA promoter of CaMV, the coat protein promoter of TMV) can be used. Plant systems (Arabidopsis) for expression of Apo AI avaliable from SemBioSys Genetics Inc. (Calgary, Canada) may also be used. When cloning in mammalian cell systems, promoters derived from the genome of mammalian cells (e.g., metallothionein promoter) or from mammalian viruses (e.g., the adenovirus late promoter, the vaccinia virus 7.5 K promoter) can be used.

C. Purification

The peptides or peptide analogs of the disclosure can be purified by many techniques well known in the art, such as reverse phase chromatography, high performance liquid chromatography, ion exchange chromatography, size exclusion chromatography, affinity chromatography, gel electrophoresis, and the like. The actual conditions used to purify a particular peptide or peptide analog will depend, in part, on synthesis strategy and on factors such as net charge, hydrophobicity, hydrophilicity, and the like, and will be apparent to those of ordinary skill in the art.

For affinity chromatography purification, any antibody which specifically binds the peptide or peptide analog may be used.

The peptides of the present invention may optionally be acetylated at the N-terminus. The peptides of the present invention may optionally have a carboxy terminal amide. In some embodiments, the peptides of the present invention may have both an acetylated N-terminus and a carboxy terminal amide. Methods of acetylating the N-terminus or adding a carboxy terminal amide are well known to one of ordinary skill in the art.

D. Antibody Production

For the production of antibodies, various host animals, including but not limited to, rabbits, mice, rats, and the like, may be immunized by injection with a peptide or peptide analog. The peptide or peptide analog can be attached to a suitable carrier (e.g., bovine serum albumin (BSA)) by means of a side chain functional group or linker attached to a side chain functional group. Various adjuvants may be used to increase the immunological response, depending on the host species, including but not limited to, Freund's (complete and incomplete), mineral gels (e.g., aluminum hydroxide), surface active substances (e.g., lysolecithin, pluronic polyols, polyanions, and oil emulsions), keyhole limpet hemocyanin, dinitrophenol, and potentially useful human adjuvants such as BCG (bacilli Calmette-Guerin) and Corynebacterium parvum.

Booster injections can be given at regular intervals, and antiserum harvested when the antibody titer thereof, as determined semi-quantitatively, for example, by double immunodiffusion in agar against known concentrations of the antigen, begins to fall. See, e.g., Ouchterlony et al., Handbook of Experimental Immunology, Wier, D. (ed.), Chapter 19, Blackwell, 1973. A plateau concentration of antibody is usually in the range of 0.1 to 0.2 mg/ml of serum (about 12 μM). Affinity of the antisera for the antigen is determined by preparing competitive binding curves, as described, for example, by Fisher (Manual of Clinical Immunology, Ch. 42, 1980).

Monoclonal antibodies to a peptide or peptide analog may be prepared using any technique which provides for the production of antibody molecules by continuous cell lines in culture, for example the classic method of Kohler & Milstein (Nature 256:495-97, 1975), or a derivative method thereof. Briefly, a mouse is repetitively inoculated with a few micrograms of the selected protein immunogen (e.g., a peptide or peptide analog) over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as enzyme-linked immunosorbent assay (ELISA), as originally described by Engvall (Meth. Enzymol., 70:419-39, 1980), or a derivative method thereof. Selected positive clones can be expanded and their monoclonal antibody product harvested for use. Detailed procedures for monoclonal antibody production are described in Harlow and Lane, Using Antibodies: A Laboratory Manual, CSHL, New York, 1999. Polyclonal antiserum containing antibodies can be prepared by immunizing suitable animals with a polypeptide comprising at least one peptide or peptide analog, which can be unmodified or modified, to enhance immunogenicity.

Antibody fragments may be used in place of whole antibodies and may be readily expressed in prokaryotic host cells. Methods of making and using immunologically effective portions of monoclonal antibodies, also referred to as “antibody fragments,” are well known and include those described in Better & Horowitz, Methods Enzymol. 178:476-96, 1989; Glockshuber et al., Biochemistry 29:1362-67, 1990; and U.S. Pat. Nos. 5,648,237 (Expression of Functional Antibody Fragments); 4,946,778 (Single Polypeptide Chain Binding Molecules); and 5,455,030 (Immunotherapy Using Single Chain Polypeptide Binding Molecules), and references cited therein. Conditions whereby a polypeptide/binding agent complex can form, as well as assays for the detection of the formation of a polypeptide/binding agent complex and quantitation of binding affinities of the binding agent and polypeptide, are standard in the art. Such assays can include, but are not limited to, Western blotting, immunoprecipitation, immunofluorescence, immunocytochemistry, immunohistochemistry, fluorescence activated cell sorting (FACS), fluorescence in situ hybridization (FISH), immunomagnetic assays, ELISA, ELISPOT (Coligan et al., Current Protocols in Immunology, Wiley, NY, 1995), agglutination assays, flocculation assays, cell panning, etc., as are well known to one of skill in the art.

E. Peptide Reconstitution

The peptides of the present invention may be reconstituted in any pharmaceutically acceptable carrier before use or administration. In one embodiment, the peptides may be reconstituted with saline, a lipid or a phospholipid, or a combination thereof. Some phospholipids that may be employed include but are not limited to the following: dipalmitoylphosphatidylcholine (DPPC); dioleoylphosphatidylcholine (DOPC); 1-palmitoyl-2-oleoylphosphatidylcholine (POPC); 1-palmitoyl-2-linoleoylphosphatidylcholine (PLPC); 1-palmitoyl-2-arachidonylphosphatidylcholine (PAPC); 1-palmitoyl-2-docosahexanoylphosphatidylcholine (PDPC); and, PMLC. DPPC, DOPC have been used to reconstitute peptides (Shah et al., Circulation. 2001 Jun. 26; 103(25):3047-50.)

The peptides of the present invention may be complexed with lipids or phospholipids in weight ratios ranging from 1:0.5 to 1:10, or 1:1 to 1:5. Any ratio within these ranges may be employed.

The phospholipids may also be complexed with other agents, such as sphingomyelin before complexing with the peptides of the present invention. Ratios of phospholipids to sphingomyelin include ratios occurring in the ranges of 1:9 to 9:1, 1:5 to 5:1, 1.2 to 2.1 (all weight %). In some embodiments, the ratio of phospholipids to sphingomyelin may be 1:1.

The peptides of the present invention may be complexed with the combination of phospholipid:sphingomyelin in weight ratios ranging from 1:0.5 to 1:10, or 1:1 to 1:5. Any ratio within these ranges may be employed.

The peptides of the present invention may be complexed with a combination of DPPC and sphingomyelin such that the final ratio of DPPC:sphingomyelin:peptide may be equal to or in a range of 1:1:1 to 6:6:1, 2:2:1 to 5:5:1, or 3:3:1 to 4:4:1.

### VI. Pharmaceutical Compositions and Uses Thereof

The peptides or peptide analogs of the disclosure can be used, alone or in combination, together with a pharmaceutically acceptable carrier, to treat any disorder in animals, especially mammals (e.g., humans), for which promoting lipid efflux and/or decreasing inflammation is beneficial. Such conditions include, but are not limited to, hyperlipidemia (e.g., hypercholesterolemia), cardiovascular disease (e.g., atherosclerosis), cerebrovascular disease, restenosis (e.g., atherosclerotic plaques), peripheral vascular disease, acute coronary syndrome, reperfusion myocardial injury, and the like. The peptides or peptide analogs of the disclosure can also be used alone or in combination during the treatment of thrombotic stroke, infarcts secondary to occlusion of a vessel and during thrombolytic treatment of occluded coronary artery disease. The peptides or peptide analogs of the disclosure can be used to treat tissue following hypoxia, ischemia and infarction due to impairment of blood supply, and also following hemorrhage following rupture or trauma of a blood vessel. Such tissue includes, without limitation, neural tissue in the central or peripheral nervous system, peripheral vascular tissue, and cardiac muscle.

It is to be understood that a mixture of peptides may include different amounts of the individual peptides. For example, in one embodiment, each peptide component of the combination may be present in a different relative percentage than each other peptide component due to differences in relative efficacy to promote lipid efflux or to provide one or more types of anti-inflammatory activity. In one exemplary embodiment, one or more of the peptides shown in SEQ ID NOs: 72, 80, 89, 354, 355 and 372, may be combined in a mixture for administration.

The peptides or peptide analogs can be used alone or in combination therapy with other lipid lowering compositions or drugs and/or other anti-inflammatory compositions or drugs used to treat the foregoing conditions. Such therapies include, but are not limited to simultaneous or sequential administration of the drugs involved. For example, in the treatment of hypercholesterolemia or atherosclerosis, the peptide or peptide analog formulations can be administered with any one or more of the cholesterol lowering therapies currently in use, for example, bile-acid resins, niacin, statins, fat uptake inhibitors, and HDL raising drugs.

In another embodiment, the peptides or peptide analogs can be used in conjunction with statins or fibrates to treat hyperlipidemia, hypercholesterolemia and/or cardiovascular disease, such as atherosclerosis. In yet another embodiment, the peptides or peptide analogs of the disclosure can be used in combination with an anti-microbial agent and/or an anti-inflammatory agent, such as aspirin. In another embodiment peptides or peptide analogs of the disclosure can be used in combination with anti-hypertensive medicines known to one of ordinary skill in the art. It is to be understood that more than one additional therapy may be combined with administration of the peptides or peptide analogs of the disclosure.

In a further embodiment, the peptides can also be expressed in vivo, by using any of the available gene therapy approaches.

In yet another embodiment, the peptides or peptide analogs can be used in conjunction with medicines used to treat patients with cerebrovascular and cardiovascular disease resulting in hypoxia, ischemia and infarction due to impairment of blood supply, and also following hemorrhage following rupture or trauma of a blood vessel. Such medicines are commonly known to one of ordinary skill in the art and include without limitation, modulators of excitatory amino acids and modulators of platelet aggregation.

A. Administration of Peptides or Peptide Analogs

In some embodiments, peptides or peptide analogs can be isolated from various sources and administered directly to the animal or human. For example, a peptide or peptide analog can be expressed in vitro, such as in an E. coli expression system, as is well known in the art, and isolated in amounts useful for therapeutic compositions. The peptide or peptide analogs of the present invention may also be made though peptide synthetic methods known to one of ordinary skill in the art, such as solid phase synthesis.

In exemplary applications, therapeutic compositions comprising the peptide or peptide analogs in an acceptable carrier are administered to an animal or a human suffering from a dyslipidemic or vascular disorder, such as hyperlipidemia, hyperlipoproteinemia, hypercholesterolemia, hypertriglyceridemia, HDL deficiency, apoA-I deficiency, coronary artery disease, atherosclerosis, stroke, ischemia, infarction, myocardial infarction, hemorrhage, peripheral vascular disease, restenosis, acute coronary syndrome, or reperfusion myocardial injury, in an amount sufficient to inhibit or treat the dyslipidemic or vascular disorder. Amounts effective for this use will depend upon the severity of the disorder and the general state of the subject's health. A therapeutically effective amount of the compound is that which provides either subjective relief of a symptom(s) or an objectively identifiable improvement as noted by the clinician or other qualified observer.

A peptide or peptide analog can be administered by any means known to one of skill in the art (see, e.g., Banga, “Parenteral Controlled Delivery of Therapeutic Peptides and Proteins,” in Therapeutic Peptides and Proteins, Technomic Publishing Co., Inc., Lancaster, Pa., 1995), such as by intramuscular, intraperitoneal, subcutaneous, or intravenous injection, but even oral, nasal, or anal administration is contemplated. In one embodiment, administration is by subcutaneous or intramuscular injection. To extend the time during which the peptide or peptide analog is available to inhibit or treat a dyslipidemic or vascular disorder, the peptide or peptide analog can be provided as an implant, an oily injection, or as a particulate system. The particulate system can be a microparticle, a microcapsule, a microsphere, a nanoparticle, or similar particle (Banga, “Parenteral Controlled Delivery of Therapeutic Peptides and Proteins,” in Therapeutic Peptides and Proteins, Technomic Publishing Co., Inc., Lancaster, Pa., 1995). The peptide or peptide analog may also be applied to a medical device for delivery to a specific location. For example, a surgical tool, catheter, stent, balloon, electrode, suture, or an artificial vessel or transplanted vessel may contain or be coated with the peptide or peptide analog.

It is to be understood that in some embodiments, one or more of the amino acids of the peptides of the present invention are D amino acids. In one embodiment, the N-terminal amino acid, the C-terminal amino acid or both are D amino acids. The presence of these D amino acids can help protect against peptide degradation. In another embodiment, all the amino acids of the peptides of the present invention are D amino acids. This embodiment is useful for protection against degradation following oral administration of a pharmaceutical composition comprising the peptides of the present invention.

In one specific, non-limiting example, a peptide is administered that includes one or more of the amino acid sequences disclosed herein.

B. Representative Methods of Administration, Formulations and Dosage

The provided peptides or peptide analogs, constructs, or vectors encoding such peptides, can be combined with a pharmaceutically acceptable carrier (e.g., a phospholipid or other type of lipid) or vehicle for administration to human or animal subjects. As described previously in the application, the peptides may be reconstituted with acceptable carriers such as saline, lipid, phospholipid, lipid:sphingomyelin complexes and phospholipid: sphingomyelin complexes. In some embodiments, more than one peptide or peptide analog can be combined to form a single preparation. The peptides or peptide analogs can be conveniently presented in unit dosage form and prepared using conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of a sterile liquid carrier, for example, water for injections, immediately prior to use. Extemporaneous injection solutions and suspensions may be prepared from sterile powders, granules and tablets commonly used by one of ordinary skill in the art.

In certain embodiments, unit dosage formulations are those containing a dose or unit, or an appropriate fraction thereof, of the administered ingredient. It should be understood that in addition to the ingredients particularly mentioned above, formulations encompassed herein may include other agents commonly used by one of ordinary skill in the art.

The pharmaceutical compositions provided herein, including those for use in treating dyslipidemic and vascular disorders, may be administered through different routes, such as oral, including buccal and sublingual, rectal, parenteral, aerosol, nasal, intramuscular, intraperitoneal, intravascular, subcutaneous, intradermal, and topical. They may be administered in different forms, including but not limited to solutions, emulsions and suspensions, microspheres, particles, microparticles, nanoparticles, and liposomes. In one embodiment, peptides or peptide analogs with suitable features of lipid efflux and low cytotoxicity can be precomplexed with phospholipids or other lipids into either discoidal or spherical shape particles prior to administration to subjects.

In another embodiment, it may be desirable to administer the pharmaceutical compositions locally to the area in need of treatment. This maybe achieved by, for example, and not by way of limitation, local or regional infusion or perfusion during surgery, direct perfusion into a vessel, such as an atherosclerotic vessel, topical application (e.g., wound dressing, peptide coated stent), injection, catheter, suppository, or implant (e.g., implants formed from porous, non-porous, or gelatinous materials, including membranes, such as silastic membranes or fibers), and the like. In one embodiment, administration can be by direct injection at the site (or former site) of a tissue that is to be treated, such as the heart or the peripheral vasculature. In another embodiment, the pharmaceutical compositions are delivered in a vesicle, in particular liposomes (see, e.g., Langer, Science 249:1527-1533, 1990; Treat et al., in Liposomes in the Therapy of Infectious Disease and Cancer, Lopez-Berestein and Fidler (eds.), Liss, N.Y., pp. 353-365, 1989). Combinations of administration methods may also be employed such as a systemic or local infusion of a peptide of the present invention, before, after or during placement of a stent coated with a peptide of the present invention.

In yet another embodiment, the pharmaceutical compositions can be delivered in a controlled release system. In one embodiment, a pump can be used (see, e.g., Langer Science 249:1527-1533, 1990; Sefton Crit. Rev. Biomed. Eng. 14:201-240, 1987; Buchwald et al., Surgery 88:507-516, 1980; Saudek et al., N. Engl. J. Med. 321:574-579, 1989). In another embodiment, polymeric materials can be used (see, e.g., Ranger et al., Macromol. Sci. Rev. Macromol. Chem. 23:61-64, 1983; Levy et al., Science 228:190-192, 1985; During et al., Ann. Neurol. 25:351-356, 1989; and Howard et al., J. Neurosurg. 71:105-112, 1989). Other controlled release systems, such as those discussed in the review by Langer (Science 249:1527-1533, 1990), can also be used.

The amount of the pharmaceutical compositions that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each subject's circumstances. An example of such a dosage range is 0.1 to 200 mg/kg body weight in single or divided doses. Another example of a dosage range is 1.0 to 100 mg/kg body weight in single or divided doses.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the subject undergoing therapy.

The pharmaceutical compositions of the present disclosure can be administered at about the same dose throughout a treatment period, in an escalating dose regimen, or in a loading-dose regime (e.g., in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. The volume of administration will vary depending on the route of administration. By way of example, intramuscular injections may range from about 0.1 ml to about 1.0 ml. Those of ordinary skill in the art will know appropriate volumes for different routes of administration.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

The subject matter of the present disclosure is further illustrated by the following non-limiting Examples.

### Example 1

### Lipid Efflux from Cells Mediated by Peptides of the Present Invention

This example demonstrates a method to test the ability of peptides of the present invention to efflux lipid from ABCA1-expressing cells.

HeLa cells stably transfected with human ABCA1 cDNA (ABCA1 cells) and HeLa cells transfected with only a hygromycin-resistant control plasmid (control cells) are produced and grown in α-modified Eagle's medium (aMEM) plus 10% fetal calf serum, as described by Remaley et al. (Biochem. Biophys. Res. Commun. 280:818-823, 2001). Cholesterol and phospholipid efflux is performed for 18 hours on noncholesterol-loaded cells radiolabeled with either cholesterol or choline (Remaley et al., Arterioscler. Thromb. Vasc. Biol. 17:1813-1821, 1997). Percentage efflux is calculated after subtracting the radioactive counts in the blank media (aMEM plus 1 mg/ml of BSA), and expressed as the percent of total radioactive counts removed from the cells during the efflux period.

Cell fixation is performed by a 10 minute treatment with 3% paraformaldehyde in phosphate buffered saline (PBS), followed by three washes with blank media. Lactate dehydrogenase (LDH) release from cells into the media is measured enzymatically (Roche Diagnostics, Indianapolis, Ind.) and expressed, after subtraction of LDH released into blank media, as the percentage of total cell LDH. Total cell LDH is determined after cell solubilization with 1% Triton X-100.

The peptides of the present invention are synthesized by a solid-phase procedure, using a Fmoc/DIC/HOBt protocol on a Biosearch 9600 peptide synthesizer (Applied Biosystems, Foster City, Calif.), or an equivalent instrument. Both L-amino acid and D-amino acid enantiomers are synthesized. All peptides are purified to greater than 98% homogeneity by reverse-phase HPLC on an Aquapore RP-300 column, or similar chromatographic procedure.

ABCA1 cells are used to assess the ability of apoA-I and synthetic peptides to efflux lipid from cells. As previously described (Hamon et al., Nat. Cell Biol. 2:399-406, 2000 and Remaley et al., Biochem. Biophys. Res. Commun. 280:818-823, 2001), control cells do not efflux significant amounts of cholesterol and phospholipid to apoA-I, but do so after transfection with ABCA1. The peptides of the present invention efflux approximately 2- to 4-fold more cholesterol and phospholipid from ABCA1 cells than from control cells. Both the peptides of the present invention and apoA-I began to show saturation for lipid efflux at approximately the same protein concentration of 10 μg/ml. The peptides of the present invention remove cholesterol and phospholipids from control cells comparable to or more than that removed by Apo A-I. In one test, SEQ ID NOs: 72, 80, 89, 354, 355 and 372 are evaluated individually and are found to remove cholesterol and phospholipids from control cells comparable to or more than that removed by Apo A-I.

### Example 2

### Lipid Efflux Time Course

This example demonstrates the cholesterol efflux time course from ABCA1-expressing cells to apoA-I and peptides of the present invention.

Cholesterol efflux from ABCA1 cells to apoA-I is first detectable after 2 hours and increases throughout the 30 hour efflux period. In contrast, there is no significant increase above background in cholesterol efflux to apoA-I from control cells. Overall, the kinetics for cholesterol efflux to peptides of the present invention from ABCA1 cells is similar to that of apoA-I, except that cholesterol efflux is first detectable after 30 minutes. The peptides of the present invention, unlike apoA-I, also promote cholesterol efflux from control cells but at a lower rate. In one test, SEQ ID NOs: 72, 80, 89, 354, 355 and 372 are evaluated individually and are found to promote cholesterol efflux from control cells but at a lower rate.

### Example 3

### Identification of Non-Cytotoxic Peptides that Promote ABCAl-Dependent Lipid Efflux

This example illustrates a method for identifying non-cytotoxic peptides that promote ABCA1-dependent lipid efflux from cells.

Peptide Design: Based on the principles and procedures described in the present application, an amino acid sequence can be designed for a peptide that promotes lipid efflux.

Peptide production: Peptides to be tested can be produced synthetically or by recombinant DNA methods, as described in the present application, and purified by reverse phase HPLC or other suitable techniques well known to one of skill in the art.

Peptide Cytotoxicity Testing: Peptides can be tested for cytotoxicity by any number of methods well known to one of skill in the art, such as the release of intracellular LDH.

Peptide ABCA1-specificity for Lipid Efflux: Peptides to be tested can be added to serum-free cell culture media in the approximate concentration range of 1-20 micrograms and incubated with a control cell line that does not express the ABCA1 transporter and the same cell line after transfection with human cDNA for the ABCA1 transporter, as described herein. Alternatively, cells, such as macrophages, that either express or do not express the ABCA1 transporter depending on their cholesterol content and/or exposure to agents that induce the ABCA1 transporter (e.g., cAMP and LXR agonists) can also be used. After a suitable period of approximately 4 to 24 hours, the conditioned media can be removed from the cells and the amount of cholesterol and or phospholipid effluxed can be quantified, as described herein. ABCA1-specific lipid efflux is calculated by subtracting the total lipid efflux of the cell line that does not express the ABCA1 transporter from the lipid efflux from the ABCA1 expressing cell line.

### Example 4

### Peptides of the Present Invention Reduce Atherosclerosis in Animal Models

The ability of the peptides of the present invention and associated fragments are tested in apoE knockout mice on a chow diet and LDL receptor knockout mice on a western high fat diet to determine the effect of these peptides to reduce atherosclerosis in a mouse model system. One or more of the peptides of the present invention, in a range of concentration of 2 mg/kg to 50 mg/kg, is injected intravenously (iv) or intraperitoneally (ip) 2 to 3 times per week over a period of approximately 6 weeks. Aortic atherosclerosis is quantitated in the aortic arch before administration of the peptides and after the 6 week period of administration. (Wu et al., J. Biol. Chem.; 2004: 279, 22913-22925). The results demonstrate reduced atherosclerosis in the aortic arch in mice in both treatment groups. In one test, SEQ ID NOs: 72, 80, 89, 354, 355 and 372 are evaluated individually and reduce atherosclerosis in the aortic arch.

### Example 5

### Administration of the Peptides of the Present Invention to Treat Atherosclerosis in Humans

Individuals with acute coronary syndrome and documented atherosclerosis have a cardiac catherization with intravascular ultrasound (IVUS) to document coronary atherosclerosis of 20 to 50% obstruction in the target artery. Each individual is on stable hypolipidemic drug therapy and receives an acceptable dose of a peptide of the present invention and/or an associated fragment iv weekly for a period of 5 to 8 weeks. A repeat IVUS measurement is made at the end of the treatment period to assess the effect of the peptide infusion on coronary atherosclerosis in the target vessel. Plaque is reduced in the atherosclerotic coronary artery following the peptide treatment demonstrating efficacy of the peptides of the present invention to treat atherosclerosis. In one test, SEQ ID NOs: 72, 80, 89, 354, 355 and 372 are evaluated individually and reduce plaque in the atherosclerotic coronary artery.

### Example 6

### Administration of the Peptides of the Present Invention to Prevent or Delay the Onset of Atherosclerosis in Humans

Individuals with documented risk factors for atherosclerosis and having high plasma cholesterol levels have a ultrasound analysis of the coronary (IVUS), carotid (IMT) or popliteal arteries to establish a baseline measurement. A portion of these individuals are daily administered individual peptides of the present invention at a dose of 2 mg/kg to 50 mg/kg intravenously (iv) or intramuscular (im) 1 to 3 times per week over a period of approximately one to six months. The other individuals receive a control peptide. A new ultrasound analysis at the end of the treatment period indicates higher levels of plaque in the vessels of individuals receiving the control peptide. This example indicates that the individual peptides of the present invention are effective in preventing or reducing atherosclerosis in individuals at risk for developing atherosclerosis and in reducing plaque accumulation in coronary, carotid or popliteal arteries.

### Example 7

### Administration of the Peptides of the Present Invention on Stents to Reduce Inflammation and Restenosis

Individuals with acute coronary syndrome and having plaque in coronary vessels which require a stent to reduce the obstruction receive an IVUS procedure to document the coronary anatomy. A representative protocol divides these individuals into three groups. One group receives a stent coated with a peptide of the present invention. A second group receives an iv infusion of a peptide of the present invention at a dose of 2 mg/kg to 50 mg/kg, 1 to 3 times per week over a period of approximately 5 to 10 weeks. A third group receives a stent coated with a peptide of the present invention and an iv infusion of a peptide of the present invention at a dose of 2 mg/kg to 50 mg/kg, 1 to 3 times per week over a period of approximately 5 to 10 weeks.

All individuals receive a second IVUS procedure at the end of 5 or 10 weeks. The results demonstrate that individuals receiving either a peptide coated stent, a peptide coated stent plus iv peptide infusion, or iv peptide infusion alone, all display reduced inflammation and restenosis when compared to their condition at the time of the first IVUS procedure.

### Example 8

### Blockade of ICAM-1/LFA-1 Mediated T-cell Adhesion to Caco-2 Cell Monolayers by the Peptides of the Present Invention

The ability of the peptides of the present invention and associated fragments are tested to decrease inflammation by their ability to block the binding of ICAM-1 to LFA-1 using a model cell adhesion assay of T cells (Mott-3) and Caco-2 cells (Anderson et al., Bioorganic & Medicinal Chemistry Letters; 2004:14, 1399-1402). Peptide concentrations of from 0 μM to 500 μM are tested. The results demonstrate dose dependent inhibition of ICAM-1/LFA-1 mediated T-cell adhesion to Caco-2 cell monolayers by the peptides of the present invention. While not wanting to be bound by the following statement, it is believed that the D domain of the peptides of the present invention are involved in this inhibitory effect. While not wanting to be bound by the following statement, it is believed in other embodiments that the A or C domains of some of the peptides of the present invention are involved in this inhibitory effect.

These results indicate that the interaction of ICAM-1 and LFA-1 in the vessel wall can be blocked by the D domain of the peptides of the present invention, and result in decreased movement of inflammatory cells, particularly T cells, from the plasma into the vessel wall. A decrease in the influx of inflammatory cells into the vessel wall decreases this inflammatory component of the atherosclerotic process and decreases the frequency of clinical vascular events (Yusuf-Makagiqansar, Inflammation: 2001; 25, 203-213).

### Example 9

### Blockade of Neutrophils Through Inhibition of the Formyl Peptide Receptor-Like-1 (FPRL1) by the Peptides of the Present Invention

The anti-inflammatory properties of the peptides of the present invention and associated fragments are tested by evaluating the peptides, and particularly the W and W″ domains, and peptides containing these domains, to block the binding of neutrophils to the formyl peptide-like 1 receptor using techniques as described by Bae et al., (Bae et al Journal of Immunology; 2004: 173, 607-614; Bae et al., Journal of Immunology; 2003: 171, 6807-6813). The peptides of the present invention are tested in a range of 1 pM to 10 μM for their ability to inhibit the binding of radiolabelled SEQ ID NO:373 Trp Lys Tyr Met Val MET peptide to FPRL1 expressing RBL-2H3 cells, and for their ability to block SEQ ID NO:373 Trp Lys Tyr Met Val MET induced cellular chemotaxis in FPRL1 expressing RBL-2H3 cells. The peptides of the present invention are also tested in other assays described in these two references by Bae et al.

The results demonstrate that the anti-inflammatory properties of the peptides of the present invention and associated fragments, including peptides containing the W domain, inhibit the binding of radiolabelled SEQ ID NO:373 Trp Lys Tyr Met Val MET peptide to FPRL1 expressing RBL-2H3 cells, inhibit SEQ ID NO:373 Trp Lys Tyr Met Val MET induced cellular chemotaxis in FPRL1 expressing RBL-2H3 cells and decrease superoxide generation.

While not wanting to be bound by the following statement, it is believed that administration of the peptides of the present invention to individuals decreases the early neutrophil influx into the vessel wall mediated by the formyl peptide-like 1 receptor in acute myocardial infarction or acute coronary syndrome resulting in a decrease in the inflammatory component of atherosclerosis, thereby reducing subsequent clinical events and post-perfusion injury.

### Example 10

### Use of Labelled Peptides of the Present Invention to Visualize and Locate Plaque in Atherosclerotic Vessels

The peptides of the present invention are complexed with phospholipids as well as gadolinium or other suitable reagent and the recombined particle is targeted to cholesterol filled cells which have increased expression of the ABCA1 transporter in the vulnerable plaque of the coronary artery. It is believed that the peptides of the present invention have a high affinity for the ABCA1 transporter and are anticipated to bind to only those cells with an increased intracellular level of cholesterol which induced upregulation of the ABCA1 transporter.

These studies on the peptides of the present invention and associated fragments are compared to results from studies employing ApoA-I protein/phospholipid complex to determine the specificity and selectivity of the peptides of the present invention versus ApoA-I in the localization of the label to vulnerable plaque. The use of the labeled peptides of the present invention to visualize vulnerable plaque provides a valuable tool for diagnosis and treatment of patients at risk for developing cardiovascular disease. (Frias et al., J Am Chem Soc; 2004:126, 16316-7).

### Example 11

### Analysis of SR-B1 Mediated Efflux and ABCA1 Mediated Efflux

The methods employed in this study have been described in U.S. Pat. Nos. 7,029,863, 7,060,452, U.S. Patent Application Publication No. 2005/0191715, and in Moya et al., Arteriosclerosis & Thrombosis 1994:14:1056-1065 and Liu et al., J. Biol. Chem., 2003:278(44), 42976-42984. SR-B1 mediated cholesterol efflux is examined in FU5AH rat hepatoma cells and ABCA1 mediated cholesterol efflux is examined in J774 mouse macrophage cells as described in these references.

TABLE 1
Controls for Efflux Assay with Samples minus blank

| | SR-BI | ABCA1 | | |
|---|---|---|---|---|
| | Mediated | Mediated | | |
| | Efflux | Efflux | +ABCA1 | −ABCA1 |
| Controls | % Per 4 h | % Per 4 h | Cells | Cells |
| 2% Human Serum | 8.47 | 12.56 | 24.658 + 0.130 | 12.100 + 0.485 |
| Pool | | | | |
| Apo A-I | 0.26 | 18.97 | 20.682 + 0.724 | 1.713 + 0.409 |
| @ 20 μg/ml | | | | |

* Efflux for all peptide samples is run at 30 μg/ml. In one test, SEQ ID NOs: 72, 80, 89, 354, 355 and 372 are evaluated.

The results demonstrate that many of the peptides of the present invention, including SEQ ID NOs: 72, 80, 89, 354, 355 and 372, each stimulate efflux of cholesterol from J774 macrophage cells (ABCA1 pathway) while having negligible or no effect on cholesterol efflux from the Fu5AH cells (SRB1 pathway), similar to the effect of Apo AI. These selective effects of these peptides demonstrates their efficacy to act as Apo A-I mimetics and selectively efflux cholesterol from cells.

These effects are also dose dependent with increasing efflux activity demonstrated through the range of 5 ug/ml to 30 ug/ml. Based on these in vitro efflux studies, the elevation of the Apo AI mimetic peptides of the present invention, in plasma, is expected to decrease coronary and other forms of atherosclerosis in high risk patients.

### Example 12

### Effect of the Peptides of the Present Invention on CD11b Expression in Monocytes Methods

Monocyte Isolation Peripheral whole blood (PWB) is drawn from healthy consenting individuals into syringes containing sodium citrate (final concentration—19.2 mM). Resting human monocytes are isolated from PWB by density centrifugation with Lymphoprep (Axis Shield). Mononuclear cells (MNCs) are collected and monocytes are further separated to purity using the Dynal negative isolation kit (Invitrogen). Monocytes are resuspended in phosphate buffered saline (PBS) and cell number is determined counting cell suspension on an automated hematology analyzer (Sysmex, KX-21N, USA).
Purification of HDL and apoA-1 Human plasma apoA-1 is isolated as previously described and the purity determined using total mass spectrometry.
Flow Cytometry 100 μL of monocytes are stimulated with either 1 μmol/L phorbol-myristate-acetate (PMA) or 1 μg/ml lipopolysaccharide (LPS) (Sigma, Australia) in the presence or absence of apoA-1 (20 μg/ml), or 20 μg/ml of the test peptides of the present invention. SEQ ID NOs: 72, 80, 89, 354, 355 and 372 are also tested individually. The cells are incubated with the FITC conjugated antibody to either the active epitope of CD11b (eBiosciences, USA, Clone CBRM1/5) or total CD11b (Serotec, USA, Clone ICRF44) for 15 min at 37° C. Cells are then fixed with 4% paraformaldehyde. Samples are controlled for by using the appropriately matched isotype matched negative control (FITC-anti-mouse IgG) (Serotec, USA, Clone W3/25). CD11b expression is measured by flow cytometry using FACS Calibur (Becton Dickinson). Analysis is conducted using the Cell Quest Pro software.
Statistical Analysis Values are presented as the mean ±SD or percentage of control ±SD. FACS results are analyzed for statistical significance using one-way ANOVA followed by Bonferroni post-hoc test. Significance is accepted at P<0.05.
Results As expected, ApoA1 (SEQ ID NO:39) significantly reduce PMA induces CD11b expression. Several of the peptides of the present invention, including but not limited to SEQ ID NOs: 72, 80, 89, 354, 355 and 372 significantly reduce PMA induced CD11b expression. Several of the peptides of the present invention, including but not limited to SEQ ID NOs: 72, 80, 89, 354, 355 and 372 significantly reduce the inflammatory response measured as a reduction in CD11b expression. These results demonstrate that several of the peptides of the present invention, including but not limited to SEQ ID NOs: 72, 80, 89, 354, 355 and 372 inhibit PMA induced CD11b expression. Taken together, the results demonstrate the anti-inflammatory properties of these peptides of the present invention. The combined effects of increasing cholesterol efflux and decreasing inflammation indicate that the peptides of the present invention effectively mimic the function of Apo AI and will decrease atherosclerosis.

### Example 13

### Evaluation of Peptide Utility in ApoE Knockout and LDL Receptor Knockout Mice

ApoE knockout and LDL receptor knockout mice, two well established animal models for the study of atherosclerosis, are injected with either saline as control or the synthetic peptides of the present invention to ascertain if these peptides can be used to increase HDL and decrease atherosclerosis.

The mice receive 3 injections per week for either 4-6 or 8-10 weeks. After the completion of the injection, the amount of hardening of the arteries or atherosclerosis is determined in the control injected animals and peptide injected animals to determine if the injections of the synthetic peptide decreased development of atherosclerosis.

The proposed studies test if intraperitoneal infusions of the apoA-I mimetic peptides of the present invention result in decreased aortic atherosclerosis in apoE and LDL receptor knockout mice, two well established mouse models of atherosclerosis.

The mouse is ideal animal specie for the proposed study since well characterized and established mouse models of atherosclerosis are readily available. In particular, apoE and LDL receptor knockout mouse models have been universally employed as animal models for atherosclerosis. Because they are available with a homogenous genetic background, these knockout mice are ideal models for analysis of atherosclerotic lesion formation which is readily impacted by genetic background variability. Additionally, lesion development in apoE and LDL-receptor knockout mice is readily modified by changes in plasma lipoproteins, including HDL, the levels of which are altered by the peptide infusion in this study.

The knockout mouse model is a well established and widely employed animal model for the study of atherosclerosis. Mice are used because of their homogenous genetic background and are ideal models for analysis of atherosclerotic lesion formation which is readily impacted by genetic background variability. Importantly, lesion development in apoE and LDL-receptor knockout mice is highly affected by changes in LDL, HDL and other plasma lipoproteins.

The peptides of the present invention are synthesized according to standard synthetic techniques. The peptides are purified for study by high pressure liquid chromatography. Some peptides are N-acetylated and/or C-terminally amidated. Several of the peptides of the present invention, including but not limited to SEQ ID NOs: 72, 80, 89, 354, 355 and 372 are tested individually.

### Mouse Models of Atherosclerosis

Four to six week old C57B1/6 mice, apoE knockout (JAX 2052) and LDL-receptor knockout (JAX 2207) mice, all in the C57B1/6 background, are obtained from Jackson Laboratories. During the entire study, C57B1/6 and apoE knockout mice are maintained on a regular chow diet (0.02% cholesterol, 3% fat) and LDL-receptor mice are maintained on a Western diet (TD88137; Harlan Teklad; Madison, Wis. —containing 0.20% cholesterol and 21% fat).

### Infusion of Synthetic ApoA-I Mimetic Peptides

Three different infusion studies are conducted.

Aim A (Infusion Study A) to determine the functional half-life of the injection of the synthetic peptide on plasma HDL levels.

In the first study (Infusion Study A), C57B1/6 mice as well as apoE knockout and LDL receptor knockout mice are injected by the intraperitoneal (ip) route or intravenous (iv) route with synthetic peptides of the present invention mimetic (30 mg/kg) on up to four different occasions two weeks apart. To evaluate changes in the plasma lipid and lipoprotein profile associated with injection of the synthetic peptide, blood for lipid analyses is obtained before and at 2, 4, 6, 24 and 48 hours after peptide injection. At the end of the study the animals are sacrificed.

Aim B (Infusion Study B) To determine whether ip injection of the synthetic peptide 3×/wk decreases development of atherosclerosis when assayed 4-5 weeks after initiation of treatment.

Aim C (Infusion Study C) To determine whether ip injection of the synthetic peptide 3×/wk decreases development of atherosclerosis when assayed 8-10 weeks after initiation of treatment.

For infusion studies B and C, mice are injected ip with either placebo or a synthetic peptide of the present invention (30 mg/kg) three times per week for either 4 to 5 weeks (Infusion Study B) or 8 to 10 weeks (Infusion Study C). Blood for lipid and lipoprotein analyses is obtained at the beginning of the study (day 0) and every two weeks after placebo/peptide injection and at the completion of the study. At the completion of the study (4 to 5 weeks for Infusion Study B and 8 to 10 weeks for Infusion Study C), the animals are sacrificed, organs harvested for analyses of cholesterol content and for aortic atherosclerosis.

### Statistical Methods to Analyze Data

All statistical analyses are conducted in SAS8.2 (SAS Institute, NC). After completion of the atherosclerosis study the mean with standard deviation between the control (C57B1/6) and treated group (apoE knockout or LDL-receptor knockout) is calculated. The differences are tested by t test (PROC TTEST) and p-values less than 0.05 are considered significant. Non-parametric analysis of aortic atherosclerosis are performed by the Mann-Whitney test.

In the first infusion study (Infusion Study A), 5 C57B1/6, 5 apoE knockout and 5 LDL receptor knockout mice are injected (IP) with a synthetic peptide of the present invention and blood is obtained for lipid and lipoprotein analyses. A total of 15 mice are used for Infusion Study A.

A total of 40 mice (20 control-placebo injected and 20 study-peptide injected mice) are utilized in each of the two other infusion studies (Infusion Study B—4 to 5 weeks duration as well as Infusion Study C—8 to 10 weeks duration). Since each infusion study is conducted in two different mouse lines (i.e.: apoE-KO and LDL receptor KO), the total number of mice used for both Infusion Studies B and C is 160.

Total number of mice used for the entire protocol is 175 (five—C57B1/6, eighty five—apoE KO mice and eighty five—LDL receptor KO mice) (These animal numbers take into account an estimated 10% morbidity rate during the course of the study as well as the number of animals previously required to achieve statistical significance during analysis of the non-random distribution aortic lesion pattern that develops in mice).

For Infusion Study A, 5 four to six week old C57B1/6, apoE knockout (JAX 2052) and LDL receptor knockout (JAX 2207) control mice receive ip or iv injections of the synthetic peptide of the present invention for up to four times two weeks apart. The sequence of procedures for this study is as follows:
1) Mice are first anaesthetized by using 1-3% isoflurane by inhalation prior to each ip injection to insure appropriate and complete delivery of placebo/peptide.
2) Mice are injected with either placebo (0.2 ml saline) or apoA-I synthetic peptide (30 mg/kg in 0.2 ml saline) via either the intraperitoneal or intravenous route.
3) In order to evaluate changes in the plasma lipids and lipoproteins in the time-frame between injections, each mouse in this study group is bled from the retro-orbital sinus following administration of a topical anesthesia. before and at 2, 4, 6, 24 and 48 hours after the peptide injection. No more than 300 ul blood is drawn during this 48 hour period.
4) At the end of infusion study A all mice are sacrificed by using Avertin (2.5%, 0.011 ml/gm, ip) or ketamine (80 ug/gm, ip).

For Infusion Studies B and C, 20 control and 20 study four to six weeks old apoE knockout (JAX 2052) and LDL-receptor knockout (JAX 2207) mice receive ip injections of either placebo or the synthetic peptide of the present invention three times per week for a total of either 4-5 weeks (Infusion Study B) or 8-10 weeks (Infusion Study C).
1) Mice are first anaesthetized by using 1-3% isoflurane by inhalation prior to each IP injection to insure appropriate and complete delivery of placebo/peptide.
2) Mice are injected ip with either placebo (0.2 ml saline) or apoA-I synthetic peptide (30 mg/kg in 0.2 ml saline) on Monday, Wednesday and Friday of each study week.
3) To measure plasma lipids and lipoproteins, each mouse in the two study groups is fasted for 4 hours in the morning (7 AM to 11 AM) and then bled from the retro-orbital sinus at the start and end of the infusion study as well as every two weeks after the initial infusion for a total of either 4 weeks (Infusion Study B) or 8 weeks (Infusion Study C). No more than 300 ul blood every two weeks is obtained from each mouse.
4) At the end of Infusion Study B and C all mice are sacrificed by cervical dislocation following isoflurane anesthesia and organs are harvested for analyses of cholesterol as well as aortic atherosclerosis.
   Before intraperitoneal injections, brief inhaled analgesia will be obtained by isoflurane utilizing the E-Z Rodent Anesthesia System in the procedure room. A topical anesthetic (proparacaine) will be applied prior to obtaining blood from the retro-orbital sinus.

The results indicate that the synthetic peptides of the present invention, including but not limited to SEQ ID NOs: 72, 80, 89, 354, 355 and 372, decrease aortic atherosclerosis compared to controls.

### Example 14

### Evaluation of Peptide Utility in Rabbits

The isolated peptides of the present invention are examined for anti-inflammatory activity using an in vivo rabbit model of acute proinflammatory changes in the carotid artery. This method is explained in detail by Nicholls et al., (Circulation 2005:111, 1543-1550). Normocholesterolemic rabbits are administered the isolated peptides of the present invention iv in a dose of from 1 to 50 mg per day for 3 days, optionally contained in unilamellar vesicles of phosphatidylcholine, with only unilamellar vesicles of phosphatidylcholine with no peptide, or saline as a control. In one test, SEQ ID NOs: 72, 80, 89, 354, 355 and 372 are administered.

On the second day, after administration of the peptides, a periarterial collar is introduced around the carotid artery and filled with saline. Two days later, the rabbits are humanely sacrificed and the carotid arteries are processed and analyzed for the presence of reactive oxygen species, the infiltration of neutrophils, and the expression of adhesion proteins and chemokines. The administration of the peptides of the present invention decrease the presence of reactive oxygen species, the infiltration of neutrophils, and the expression of adhesion proteins and chemokines compared to controls, thereby demonstrating anti-inflammatory activity in vivo, which can help retard the atherogenic process.

### Example 15

### Evaluation of Peptide Utility to Promote Reverse Cholesterol Transport In Vivo

The isolated peptides of the present invention are examined for the ability to release cholesterol in mice using the method described by Zhang et al., (Circulation. 2003; 108: 661-663). Macrophages (J774 cells) are loaded with tritiated cholesterol in vitro and injected ip into mice. These mice are administered isolated peptides of the present invention, iv, at a dose of from 1 ug to 1 mg, or saline as a control. In one test, SEQ ID NOs: 72, 80, 89, 354, 355 and 372 are administered. The peptides are administered either in saline as a vehicle or in lipid vesicles, such as vesicles of phosphatidylcholine. The mice receiving the peptides of the present invention demonstrate increased levels of tritiated cholesterol in the liver, plasma and feces, than mice receiving saline. The results demonstrate that the peptides of the present invention stimulate reverse cholesterol transport from macrophages to the liver and feces.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims. It will be apparent that the precise details of the constructs, compositions, and methods described herein may be varied or modified without departing from the spirit of the described invention. We claim all such modifications and variations that fall within the scope and spirit of the claims below.

1. An isolated peptide of formula I comprising:
(A-B-C)ₙ, wherein I.
A comprises helix 2 of ApoA-I, helix 5 of ApoA-I, helix 6 of ApoA-I, helix 7 of ApoA-I, or a modified form of helix 8 of ApoA-I, or a variation or a conservative substitution thereof;
C comprises helix 8 of ApoA-I, or a variation or a conservative substitution thereof;
B comprises a linking group between A and C and is SEQ ID NO:11 Ala Lys Pro Val Leu, SEQ ID NO:13 Lys Pro Val Leu, SEQ ID NO:14 Pro Val Leu, Pro Val, Pro, SEQ ID NO:15 Ala Lys Pro Val, SEQ ID NO:12 Leu Val Pro Lys Ala, SEQ ID NO:16 Ala Lys Pro, SEQ ID NO:17 Lys Pro Val, Lys Pro, SEQ ID NO:18 Lys Leu Ser Pro Leu, SEQ ID NO:19 Leu Ser Pro Leu, SEQ ID NO:20 Ser Pro Leu; SEQ ID NO:21 Leu Pro Ser Leu Lys, SEQ ID NO:22 Leu Pro Ser Leu, or SEQ ID NO:23 Leu Pro Ser, or a conservative substitution thereof; and,
n is an integer from 1 to 10,
provided that when A is helix 5 of ApoA-I, helix 6 of ApoA-I, or a modified form of helix 8 of ApoA-I, B is not Pro, SEQ ID NO:18 Lys Leu Ser Pro Leu, SEQ ID NO:19 Leu Ser Pro Leu, SEQ ID NO:20 Ser Pro Leu, SEQ ID NO:21 Leu Pro Ser Leu Lys, SEQ ID NO:22 Leu Pro Ser Leu, or SEQ ID NO:23 Leu Pro Ser, or a conservative substitution thereof.

2. The isolated peptide of claim 1, wherein,
SEQ ID NO:1
Thr Gln Glu Phe Trp Asp Asn Leu Glu Lys Glu Thr
Glu Gly Leu Arg Gln Glu,
SEQ ID NO:2
Glu Gln Arg Leu Gly Glu Thr Glu Lys Glu Leu Asn
Asp Trp Phe Glu Gln Thr,
SEQ ID NO:3
Gly Glu Glu Met Arg Asp Arg Ala Arg Ala His Val
Asp Ala Leu Arg Thr His,
SEQ ID NO:4
His Thr Arg Leu Ala Asp Val His Ala Arg Ala Arg
Asp Arg Met Glu Glu Gly,
SEQ ID NO:5
Ser Asp Glu Leu Arg Gln Arg Leu Ala Ala Arg Leu
Glu Ala Leu Lys Glu Asn,
SEQ ID NO:6
Asn Glu Lys Leu Ala Glu Leu Arg Ala Ala Leu Arg
Gln Arg Leu Glu Asp Ser,
SEQ ID NO:7
Leu Ala Glu Tyr His Ala Lys Ala Thr Glu His Leu
Ser Thr Leu Ser Glu Lys,
SEQ ID NO:8
Lys Glu Ser Leu Thr Ser Leu His Glu Thr Ala Lys
Ala His Tyr Glu Ala Leu,
SEQ ID NO:9
Leu Glu Ser Ala Lys Val Ser Ala Leu Ser Ala Leu
Glu Glu Ala Thr Lys Lys,
or
SEQ ID NO:10
Lys Lys Thr Ala Glu Glu Leu Ala Ser Leu Ala Ser
Val Lys Ala Ser Glu Leu,
Leu, or a variation or conservative substitution thereof,
C is SEQ ID NO:24 Leu Glu Ser Phe Lys Val Ser Phe Leu Ser Ala Leu Glu Glu Tyr Thr Lys Lys, or SEQ ID NO:25 Lys Lys Thr Tyr Glu Glu Leu Ala Ser Leu Phe Ser Val Lys Phe Ser Glu Leu, or a variation or conservative substitution thereof, and
n is 1.

3. The isolated peptide of claim 1, wherein a N-terminal amino acid is acetylated and a C-terminal amino acid is amidated.

4. The isolated peptide of claim 1, further comprising one or more of D, E, F, and W, to form subgeneric formula II,
D-E-(A-B-C)ₙ-F-W, wherein II.
D is absent or present and is SEQ ID NO:26 Pro Arg Gly Gly Ser Val Leu Val Thr, or SEQ ID NO:27 Thr Val Leu Val Ser Gly Gly Arg Pro, or multiples, variations or conservative substitutions thereof;
E is absent or present and is Pro, SEQ ID NO:21 Leu Pro Ser Leu Lys, SEQ ID NO:18 Lys Leu Ser Pro Leu, SEQ ID NO:30 Pro Lys Ala, SEQ ID NO:11 Ala Lys Pro Val Leu or SEQ ID NO:16 Ala Lys Pro or a conservative substitution thereof, provided E is present if D is present;
F is absent or present and is Pro, SEQ ID NO:31 Ala Leu Ser Pro Leu, SEQ ID NO:22 Leu Pro Ser Leu Ala, SEQ ID NO:11 Ala Lys Pro Val Leu, or SEQ ID NO:12 Leu Val Pro Lys Ala or a conservative substitution thereof, provided F is present if W is present; and,
W is absent or present and is SEQ ID NO:33 Trp Arg Trp Trp Trp Trp, or SEQ ID NO:34 Trp Trp Trp Trp Arg Trp, or multiples, variations or conservative substitutions thereof.

5. The isolated peptide of claim 1, further comprising one or more of D, E, F, and W, to form subgeneric formula III,
W-E-(A-B-C)ₙ-F-D, wherein III.
D is absent or present and is SEQ ID NO:26 Pro Arg Gly Gly Ser Val Leu Val Thr, or SEQ ID NO:27 Thr Val Leu Val Ser Gly Gly Arg Pro, or multiples, variations or conservative substitutions thereof;
E is absent or present and is Pro, SEQ ID NO:21 Leu Pro Ser Leu Lys, SEQ ID NO:18 Lys Leu Ser Pro Leu, SEQ ID NO:30 Pro Lys Ala, SEQ ID NO:1 Ala Lys Pro Val Leu or SEQ ID NO:16 Ala Lys Pro or a conservative substitution thereof, provided E is present if D is present;
F is absent or present and is Pro, SEQ ID NO:31 Ala Leu Ser Pro Leu, SEQ ID NO:22 Leu Pro Ser Leu Ala, SEQ ID NO:1 Ala Lys Pro Val Leu, or SEQ ID NO:12 Leu Val Pro Lys Ala or a conservative substitution thereof, provided F is present if W is present; and,
W is absent or present and is SEQ ID NO:33 Trp Arg Trp Trp Trp Trp, or SEQ ID NO:34 Trp Trp Trp Trp Arg Trp, or multiples, variations or conservative substitutions thereof.

6. The isolated peptide of claim 1, further comprising one or more of G and H, to form subgeneric formula IV,
G-(A-B-C)ₙ-H, wherein III.
G is absent or present and is SEQ ID NO:20 Ser Pro Leu, or SEQ ID NO:23 Leu Pro Ser or a variation or a conservative substitution thereof; and,
H is absent or present and is SEQ ID NO:35 Leu Asn Thr Gln, or SEQ ID NO:36 Gln Thr Asn or a variation or a conservative substitution thereof.

7. The isolated peptide of claim 1, further comprising one or more of D, E, F, W, G and H to form subgeneric formula V,
D-E-G-(A-B-C)ₙ-H-F-W, wherein V.
D is absent or present and is SEQ ID NO:26 Pro Arg Gly Gly Ser Val Leu Val Thr, or SEQ ID NO:27 Thr Val Leu Val Ser Gly Gly Arg Pro, or multiples, variations or conservative substitutions thereof;
E is absent or present and is Pro, SEQ ID NO:21 Leu Pro Ser Leu Lys, SEQ ID NO:18 Lys Leu Ser Pro Leu, SEQ ID NO:30 Pro Lys Ala, SEQ ID NO:11 Ala Lys Pro Val Leu or SEQ ID NO:16 Ala Lys Pro, or a conservative substitution thereof, provided E is present if D is present;
F is absent or present and is Pro, SEQ ID NO:31 Ala Leu Ser Pro Leu, SEQ ID NO:22 Leu Pro Ser Leu Ala, SEQ ID NO:11 Ala Lys Pro Val Leu, or SEQ ID NO:12 Leu Val Pro Lys Ala, or a conservative substitution thereof, provided F is present if W is present; and,
W is absent or present and is SEQ ID NO:33 Trp Arg Trp Trp Trp Trp or SEQ ID NO:34 Trp Trp Trp Trp Arg Trp, or multiples, variations or conservative substitutions thereof;
G is absent or present and is SEQ ID NO:20 Ser Pro Leu, SEQ ID NO:23 Leu Pro Ser or a variation or a conservative substitution thereof, provided G is present when D is absent;
H is absent or present and is SEQ ID NO:35 Leu Asn Thr Gln, SEQ ID NO:36 Gln Thr Asn Leu or a variation or a conservative substitution thereof, provided H is present when W is absent.

8. An isolated peptide of formula VI
D-I-W, wherein VI.
D is absent or present and is SEQ ID NO:26 Pro Arg Gly Gly Ser Val Leu Val Thr or SEQ ID NO:27 Thr Val Leu Val Ser Gly Gly Arg Pro, or multiples, variations or conservative substitutions thereof;
I is a group linking D and W and is SEQ ID NO:37 Leu Ala Lys Pro Val Leu, SEQ ID NO:38 Leu Val Pro Lys Ala Leu or multiples, variations, combinations thereof or conservative substitutions thereof;
W is absent or present and is SEQ ID NO:33 Trp Arg Trp Trp Trp Trp or SEQ ID NO:34 Trp Trp Trp Trp Arg Trp, or multiples, variations or conservative substitutions thereof.

9. The isolated peptide of claim 1, further comprising a label.

10. The isolated peptide of claim 1 further comprising a pharmaceutically acceptable carrier.

11. The isolated peptide of claim 3 further comprising a pharmaceutically acceptable carrier.

12. The isolated peptide of claim 4 further comprising a pharmaceutically acceptable carrier.

13. The isolated peptide of claim 5 further comprising a pharmaceutically acceptable carrier.

14. The isolated peptide of claim 6 further comprising a pharmaceutically acceptable carrier.

15. The isolated peptide of claim 7 further comprising a pharmaceutically acceptable carrier.

16. The isolated peptide of claim 8 further comprising a pharmaceutically acceptable carrier.

17. A method of treating disease in an animal or a human comprising administering to the animal or the human a composition comprising an isolated peptide of formula I comprising:
(A-B-C)ₙ, wherein I.
A comprises helix 2 of ApoA-I, helix 5 of ApoA-I, helix 6 of ApoA-I, helix 7 of ApoA-I, or a modified form of helix 8 of ApoA-I, or a variation or a conservative substitution thereof;
C comprises helix 8 of ApoA-I, or a variation or a conservative substitution thereof;
B comprises a linking group between A and C and is SEQ ID NO:11 Ala Lys Pro Val Leu, SEQ ID NO:13 Lys Pro Val Leu, SEQ ID NO:14 Pro Val Leu, Pro Val, Pro, SEQ ID NO:15 Ala Lys Pro Val, SEQ ID NO:12 Leu Val Pro Lys Ala, SEQ ID NO:16 Ala Lys Pro, SEQ ID NO:17 Lys Pro Val, Lys Pro, SEQ ID NO:18 Lys Leu Ser Pro Leu, SEQ ID NO:19 Leu Ser Pro Leu, SEQ ID NO:20 Ser Pro Leu; SEQ ID NO:21 Leu Pro Ser Leu Lys, SEQ ID NO:22 Leu Pro Ser Leu, or SEQ ID NO:23 Leu Pro Ser, or a conservative substitution thereof; and,
n is an integer from 1 to 10,
provided that when A is helix 5 of ApoA-I, helix 6 of ApoA-I, or a modified form of helix 8 of ApoA-I, B is not Pro, SEQ ID NO:18 Lys Leu Ser Pro Leu, SEQ ID NO:19 Leu Ser Pro Leu, SEQ ID NO:20 Ser Pro Leu, SEQ ID NO:21 Leu Pro Ser Leu Lys, SEQ ID NO:22 Leu Pro Ser Leu, or SEQ ID NO:23 Leu Pro Ser, or a conservative substitution thereof; and,
a pharmaceutically acceptable carrier.

18. A method of treating disease in an animal or a human comprising administering to the animal or the human the pharmaceutical composition of claim 11.

19. A method of treating disease in an animal or a human comprising administering to the animal or the human the pharmaceutical composition of claim 12.

20. A method of treating disease in an animal or a human comprising administering to the animal or the human the pharmaceutical composition of claim 13.

21. A method of treating disease in an animal or a human comprising administering to the animal or the human the pharmaceutical composition of claim 14.

22. A method of treating disease in an animal or a human comprising administering to the animal or the human the pharmaceutical composition of claim 15.

23. A method of treating or inhibiting a dyslipidemic disorder or a vascular disorder in an animal or a human, comprising administering to the animal or the human a therapeutically effective amount of the pharmaceutical composition of claim 10.

24. A method of treating or inhibiting a dyslipidemic disorder or a vascular disorder in an animal or a human, comprising administering to the animal or the human a therapeutically effective amount of the pharmaceutical composition of claim 11.

25. A method of treating or inhibiting a dyslipidemic disorder or a vascular disorder in an animal or a human, comprising administering to the animal or the human a therapeutically effective amount of the pharmaceutical composition of claim 12.

26. A method of treating or inhibiting a dyslipidemic disorder or a vascular disorder in an animal or a human, comprising administering to the animal or the human a therapeutically effective amount of the pharmaceutical composition of claim 13.

27. A method of treating or inhibiting a dyslipidemic disorder or a vascular disorder in an animal or a human, comprising administering to the animal or the human a therapeutically effective amount of the pharmaceutical composition of claim 14.

28. A method of treating or inhibiting a dyslipidemic disorder or a vascular disorder in an animal or a human, comprising administering to the animal or the human a therapeutically effective amount of the pharmaceutical composition of claim 15.

29. A method of visualizing plaque in an animal or a human comprising:
administering the labeled peptide of claim 9 in an acceptable carrier to a vascular system of the animal or the human; and,
detecting the labeled peptide bound to the plaque within the vascular system of the animal or the human.
